SEQUENCE LISTING (92 sequences)

SEQ ID NO: 1 - protein, 886 aa, Uliginvirus njord
  source 1..886: note = Pseudomonas phage Njord
MPNIETQEALEAKMHGAGLDRFIRNNDRAITGGSASDTDWYRRLTRQFVKPMADGIQAYLDYYEGRRGKPSASLVHLRCLPTETASYIAIKTIFDGLTNPNQTAQNLSEQIGKRIEDEVRFMKLDNAAPKFIKAIKESLKRSSSQKYSHGHDVMVHAEKLLAEDKERQDKYGVMIDRHLSWPDNDIFNLGSKLIELFANNMLLDGEVLIQKRLIVRSQIDRVTVIEATPAMEKWIEEYKVVVGEMAPAFAPCVIKPLDWTGPRFGGWHTEKMRRQMPLAKCKQQSVLSRLTKKQMPRVYSAVNALQAVEWRVNHRILSVANEARLRGLPLALPDREKLATPPCPVPHMYAELRGAELMSVLSESQKEAFRTWKADAAKTYNDEAERKAKYREVCATIDQGIQYKDFDKIHFVYTLDFRGRVYCQSSLISPQGGDLQKSLCQFANGMPLGESGVHWFKVQGANVWGWDKKQFEERDELVMADDFVELCLDIAADPLTFTDWTQADKPWQFLAWCFEYADFVSHCETNPPETFVSRIAVAMDGSCSGIQHYSAMLRDEIGGKEVNLVPGDRPQDIYGAVAKIVGRWMEDISELQVPCESFTRMLSKGAVEGQFKLCPLEAQMLSRAWVEIGVTRSMTKKPVMTLPYGSSQMTCRESMAQYLNDLQKKENEQSRAQNRKPVAVHPFNDSKGSMSRMQAELFASGLTWEAIGHVVVAARAAMRFIKKVTHETSSRNVALEWIAPTGFIVNQCIFDSTPRRVKTQMMGETFFTIREENPTISVNKMKSSAAPNFVHSMDASHLCIAVDEFALAGITSIAVIHDSFGTHAGNTERMRKLLINSFVDMYQNHDVIRDFYEAQCERVKCDLELDLPAVGTLDLETVRHSKYIFA

SEQ ID NO: 2 - protein, 884 aa, Uliginvirus nerthus
  source 1..884: note = Pseudomonas phage Nerthus
MHGAGMERFVRNNERAISAGSASDTDWFRRLTRNFVKPMADGIQAYLDYYEGRRGRPSASLVHLRCMPTEVSSYIAIKTIFDGLMNPNQTAQNLAEQIGRRIEDEVRFTKLENAAPKYIKAIKESLKRQASQKYSHGHDVLVHAEKLLSEDQTRKDKYGVELERFTGWSDNDIFNLGSKLIELFANNMLLNGVPVIRKRNIHRSLRDEVVVIEATDAMEQWIEEYKVVVGEMSPAYAPCVVRPLDWTGPRMGGWHTEKMRRGMPLVKCRDHKVLSRLTRKQMPSVYAAVNALQGVEWRVNQRILQVANEARLRGLPLGLPERERLAMPPCPVPSHFAELRGKELMDVLEPEQQEAFRSWKMEAAKTYNDEAERKAKYREVCATIDQGIQYQEFERIHFVYTCDFRGRVYAQSSLISPQGGDLQKALCQFANAMALGDEGEYWFKVHGANVWGWDKLPFDERVANVSSAEFIDDCLDIAADPLTFTNWTQADSPWQFLAWCFEYADFVSHCETEAPETFLSRVAVAMDGSCSGIQHYSAMLRDEIGGKEVNLVPGDRPQDIYGAVAKIVASWMEQIVAGTVPCESFERMLAKGDRIRTAGDWESAHQAPFILNAFEAQMIAAEWLRIGVTRSLCKKPVMTLPYGSSQMTCRESMSQHLDQLQKDENERARGQFRKAVAVHNFNDGKGSMSRQQGELFASGLTWEGIGHVVVAARAAMKFIKKVTHEVGSRNTPLEWVTPTGFIVHQAIYDSTCRRVKTQMMGDTFFTLREEEPVISVNKMKSSAAPNFVHSMDASHLVLATEGFHNNAITSIAVIHDSFGTHAGNTAKLRRILTNSFVDMYQQHDVIKDFYDAQCDRVMTDLELDLPATGSLDLEAVRESKYIFA

SEQ ID NO: 3 - protein, 900 aa, Uliginvirus alpheus
  source 1..900: note = Pseudomonas phage Alpheus
MLDTQLALEAKMHGAGMERFIRNNDRAIAAGAASDTDWFRRLTRNFVKPMADGIQAYLDYYEGRRGRPSASLVHLRCMPTEVSSYIAIKCIFDGLMNPNQTAQNLAETIGRRIEDEVRFTKLENAAPKYIQAIKESLKRQSSQKYGHGHDVMVHAEKLLSEDQTRKDKFGVDLERFVGWSDNDIFNLGSKLIELFANNMLLNGVPVIQKRNIHRGVRDEVVVIEATEAMEQWIEEYKAVVGEMSPAYAPCVVRPLDWTGPRSGGWHTPKMQRGMPLVKCRDHKVLSRLTRKQMPSVYAAVNALQGVEWRVNKRILQVANEARLRGLPLALPAREKLATPPCPVPSMFSELRGKELMDVLEPEQQEAFKAWKMEAAKTYNDEAERKAKYREASATIDQGLQYQDFERMHFVYTCDFRGRVYAQSSLISPQGGDLQKALCQFANAMALGDEGEYWFKVHGANVWGWDKKEFDERVALVSEDSFIDLCLDIAADPLTFTDWTQADKPWQFLAWCLEYADFVSHCETEAPEKFLSRVAVAMDGSCSGIQHYSAMLRDEVGGKEVNLVPGTTPQDIYGAVAKIVRRWMEDIVDGHVPCESFERMLSKAAARHKAIVEDGGVWDSAMAPPFTLNALESQMIATEWLRIGVTRSLCKKPVMTLPYGSSQMTCRESMAQHLDSLQKDENDKARAQFRKPTAVHNFNDGKGSMSRMQAELFASGLTWEGIGHVVVAARAAMKFIKKVTHEVSIRNVPLEHVTPTGFIVHQAIYDSTARRVKTQMMGDTFFTIREENPQLSARAMKSSAAPNFVHSMDASHLVLATEGFSNNGIHSIAVIHDSFGTHAGNTAKLRKILVDSFVDMYQKHDVVKDFYEAQCDRVMADLELDLPATGTLDLETVRESKYIFA

SEQ ID NO: 4 - protein, 900 aa, Uliginvirus achelous
  source 1..900: note = Pseudomonas phage Achelous
MQDIQLALEAKMHGAGMDRFIRNNDRAIAGGSGSDTDWFRRLTRNFVKPMADGIQAYLDYYEGRRGRPSASLVHLRCMPTEVSSYIAIKTIFDGLMNPNQTAQGIAEQIGRRIEDEVRFTKLENAAPKYIKAIKESLKRQASQKYSHGHDVLVHAEKLLSEDQARKDKFGVDLERFTGWSDNDIFNLGGKLVELFANNMLLNGVPVIRKRNIHRSLRDEVVVLEATEVMEAWIEEYKVVVGEMSPAYAPCVVRPLDWTGPRAGGWHTEKMRRGMPLVKCRDHSVLSRLTKRQMPSVYAAVNALQGVQWRVNKRILQVANEARLRALPLGIPARERLETPPCPVPSHFAELRGQELMAVLDAQQQEDFKAWKMEAAKTYNDEAERKAKYREAAATIDQGIQYQDFEHMHFVYTCDFRGRVYAQSSLISPQGGDLQKALCQFANAMPFGASGEYWFKVHGANVWGWDKLPFDERVAKVNSDEFLDTCLDIAADPLTFTDWTQADKPWQFLAWCVEYADFVSHCETEAPETFPSRIAVAMDGSCSGIQHYSAMLRDEVGGKEVNLVPGDSPRDIYGAVAKIVQRWMEDIVEGLVPCESFERMLAKAEARMKIQQSEGGSWDSISSPPFTLNQMESRLIAAEWLTIGVTRSLCKKPVMTLPYGSSQMTCRESMSQHLSDLQKAENEKARGQFRQPVQMHNFNDGKGSMSRQQGELFASGLTWEAIGKVVVAARAAMQFIKKVTHEVSIRNHGLEWVTPTGFIVNQCVYDSTARRVKTQMMGETFFTIREENPTVSVHKMKSSSAPNFVHSMDASHLVLATEDFAENGITSIAVIHDSFGTHAGNTELLRKLLTDSFVDMYLEHDVIKEFYDAQCDRVLCDLELDLPATGTLDLEDVRNSKYIFA

SEQ ID NO: 5 - protein, 887 aa, Uliginvirus uligo
  source 1..887: note = Pseudomonas phage uligo
MNDMQQIQLDLEAKMHGAGLDRFVRNNDRAVNGGMGSDTDWYRRLTRQFVKPMADGIQAYLDYYEGRRGKPSASLVHLRCLPTEVSSYIAIKTIFDGLTNPNQTAQNLAEQIGKRIEDEVRFMKLEGAAPRFIKAIKESLKRSSSQKYSHGHDVLVHAEKLLSEDKTRQDKYGVAVDRHLSWSDNDVFNLGGKLIELFANNMLMDGEPLIQKRLIIRSATDKVNVIESTPAMDLWVEKYKVVVGEMSPAFAPCVVKPLDWTGPRFGGWHTEKMRRQMPLAKCKSQAVLSRLTQKQMPTVYRAVNALQAVEWRVHQQILSVANEARLRGLPVAMPAREKLETPPCPVPSMFNELRGAELMAVLDDDQKEAFRQWKADAAKTYNDEAERKAKYREVVATIDQGIQYKDFDRIHFVYTLDFRGRVYCQSSLISPQGGDLQKALCQFANGMALGTEGVKWFKIQGANVWGWDKKTFPERVRLVSEGDFVELCLDIAADPLTFTDWTQADKPWQFLAWCFEYAAFVSHCETEPAETFISRIAVAMDGSCSGIQHYSAMLRDEVGGREVNLVPGDAPQDIYGAVAKIVGRWMEDISELQEPCESYTRMLSKGALEGQFKLDPLEAQMLARAWLNIGVTRSMTKKPVMTLPYGSSQMTCRESIAQYLNDLQKKENDQARGQFRKPVPVHPFNETKGSMSRMQGELLASGLTWEAIGHVVVAARAAMRFIKKVTHECGIRNQHLEWVTPTGFIVNQCIFDSTARRVKTQMMGETFFTIREETKTISVNKMKSSSAPNFVHSMDASHLCIAVDDLAAHGITSIAVIHDSFGTHAGNTELLRKLLINSFVDMYLSHDVIKDFYEAQCDRMMVDLELDLPAVGTLDLETVRSSQYIFA

SEQ ID NO: 6 - protein, 928 aa, Kaohsiungvirus A318
  source 1..928: note = Vibrio phage phi-A318
MNKDLMQIQLELEAEMHGAGILRFEKNNQRALESGNGSDADWYRRLTREFVRPMADAIQAYIDYYTGRRGKPSACLMHLKCLSPEAAAYIAIKTIFDGLSNHEITAQNLANNIGRRIEDEVRFTKLNNAAPKYIQAIKESLKKRNSNSYKFDHDVMVHAEKELKLLADFQALLGAGEEKLEIMRLLSIDNDKYQHLLDKAEFAIDIDRWIPWSDNDVIQLGAKLIEIFGKNMLMDGQPLIAKQVNGQGRGKTTAKIVATDALETWIQGYKEVVGNMSPAYEPCVVPPKEWKSPFNGGYHSKEVSSNLPIAKVRDKKHLRRLTRKQMPKVYQALNALQNVKWQINPSIYAIANEIRLRELPLGMPEFKKYNKPACPVPAIYSDLRGEDLMQVLDDDQKAAFKNWKRQIASIYAKENKRKSDVREVVATLDQANKFKDFENLHFVYTLDFRGRVYCHSSLVSPQGGDLQKALIRFADSMPLGSTGAYWFKVHGANVWGWDKETFAERVSRCETEEFKEMCLDIAADPITFTDWTKADKPWQFLAWCYEYAALLEWIDEGNNEQDFPSHIAVAMDGSCSGIQHYSAMLRDSVGGKEVNLLPSEKPQDIYGAVSKVVVEWMEAITNNDERDCPIYDKLIDSLVAKFEKAKLTGDLESKARIMAYKFCEEWLRIGVTRSMTKKPVMTLPYGSSQLTCRDSIADYLKDLQEKADNKARARGMKAGSIHAFTDSDGDLPLRDAISFASMLTWAAIGEVVVAARAGMKYIKEVCSAIAKDNKPLEWMTPTGFIVKQAVYEVKEDKRIKTQMLGETFFRTSVRTDKIDPRRMASSAAPNFVHSMDASHLVLAVCAFEDNGITSIAVIHDSFGTHAGNTELLRLHLSGSFVQMYEENDVIKDFIEYNEALILTEIEATVPEKGELDLQEVLKSLYCFA

SEQ ID NO: 7 - protein, 930 aa, Kaohsiungvirus AS51
  source 1..930: note = Vibrio phage AS51
MNKDLMQIQLELEAEMHGAGILRFEKNNQRALESGNGSDADWYRRLTREFVRPMADAIQAYIDYYTGRRGKPSACLMHLKCLSPEAAAYIAIKTIFDGLSNHEITAQNLANNIGRRIEDEVRFTKLNNAAPKYIQAIKESLKKRNSNSYKFDHDVMVHAEKELKLLADFQALLGAGEEKLEIMRLLSIDNDKYQHLLDKAEFAIDIDRWIPWSDNDVIQLGAKLIEIFGKNMLMDGQPLIAKQVNGQGRGKTTAKIVATDALETWIQGYKEVVGNMSPAYEPCVVPPKEWKSPFNGGYHSKEVSSNLPIAKVRDKKHLRRLTRKQMPKVYQALNALQNVKWQINPSIYAIANEIRLRELPLGMPEFKKYNKPACPVPAIYSDLRGEDLMQVLDDDQKAAFKNWKRQIASIYAKENKRKSDVREVVATLDQANKFKDFENLHFVYTLDFRGRVYCHSSLVSPQGGDLQKALIRFADSMPLGSTGAYWFKVHGANVWGWDKETFAERVSRCETEEFKGVPVCLDIAADPITFTDWTKADKPWQFLAWCYEYAALLEWIDEGNNEQDFPSHIAVAMDGSCSGIQHYSAMLRDSVGGKEVNLLPSEKPQDIYGAVSKVVVEWMEAITNNDERDCPIYDKLIDSLVAKFEKAKLTGDLESKARIMAYKFCEEWLRIGVTRSMTKKPVMTLPYGSSQLTCRDSIADYLKDLQEKADNKARARGMKAGSIHAFTDSDGDLPLRDAISFASMLTWAAIGEVVVAARAGMKYIKEVCSAIAKDNKPLEWMTPTGFIVKQAVYEVKEDKRIKTQMLGETFFRTSVRTDKIDPRRMASSAAPNFVHSMDASHLVLAVCAFEDNGITSIAVIHDSFGTHAGNTELLRLHLSGSFVQMYEENDVIKDFIEYNEALILTEIEATVPEKGELDLQEVLKSLYCFA

SEQ ID NO: 8 - protein, 928 aa, Kaohsiungvirus Vp670
  source 1..928: note = Vibrio phage Vp670
MNKDLMQIQLELEAEMHGAGILRFEKNNQRALESGNGSDADWYRRLTREFVRPMADAIQAYIDYYTGRRGKPSACLMHLKCLSPEAAAYIAIKTIFDGLSNHEITAQNLANNIGRRIEDEVRFTKLNNAAPKYIQAIKDSLKKRNSNSYKFDHDVMVHAEKELKLLADFQALLGAGEEKLEIMRLLSIDNDKYQHLLDKAEFAIDIDRWIPWSDNDVIQLGAKLIEIFGKNMLMDGQPLIAKQVNGQGRGKTTAKIVATDALETWIQGYKEVVGNMSPAYEPCVVPPKEWKSPFNGGYHSKEVSSNLPIAKVRDKKHLRRLTRKQMPKVYQALNALQNVKWQINPSIYAIANEIRLRELPLGMPEFKKYNKPACPVPAIYSDLRGEDLMQVLDDDQKAAFKNWKRQIASVYAKENKRKSDVREVVATLDQANKFKDFENLHFVYTLDFRGRVYCHSSLVSPQGGDLQKALIRFAESMPLGSTGAYWFKVHGANVWGWDKETFAERVSRCETEEFKEMCLDIAADPITFTDWTKADKPWQFLAWCYEYAALLEWIDEGNNEQDFPSHIAVAMDGSCSGIQHYSAMLRDSVGGKEVNLLPSDKPQDIYGAVSKVVVEWMEAITNNDERDCPIYDKLIDSLVSKFEKAKLTGDLESKARIMAYKFCEEWLRIGVTRSMTKKPVMTLPYGSSQLTCRDSIADYLKDLQEKADNKARARGMKAGSIHAFTDSDGDLPLRDAISFASMLTWSAIGEVVVAARAGMKYIKEVCTAIAKDNKPLEWMTPTGFIVKQAVYEVKEDKRIKTQMLGETFFRTSVRTDKIDPRRMASSAAPNFVHSMDASHLVLAVCAFEDNGITSIAVIHDSFGTHAGNTELLRLHLSGSFVQMYEENDVIKDFIEYNEALILTEIEATVPEKGELDLQEVLKSLYCFA

SEQ ID NO: 9 - protein, 914 aa, Jhansiroadvirus gwaliVC1
  source 1..914: note = Vibrio phage Vc1
MKDLHAVQIELEEEMHGAGILRFTKNNQRSIDSGQAGETDWFRRLTREFVRPMADAIDAYKDYYAGRRGKPSATIGHLKCISSEAAGYIAIKTIFDSMAGHTTSAQALANHIGRRIEDEIRFTKLSEAAPEYIGAIKESLKKRSNQSYKFEHDSLVHAEKELKMLSDFQALHNGGVDKQEIMRLLGADEEKYKALLEKVEYAIDFDRWIPWAQNDVLQLGAKMIDIFANNMLLNGKPVVERVTISTGTASNRQTPAAIVPTESLEAWVEEYKQVMGSIAPAYEPCVVPPRPWKSPFNGGYHSKKVSSRLNMVKCPDKKHLKRLSRSQMPKVYEAVNALQEVKWQIHPSVLAVANEIRLRGLPLGMPRMDKADKPICPVPAIYSELRGAELMACLDEDQKEAFLKWKRDTVAYYASEQKRRSDVRETIATIDQGLKFKDYEALHYVYTLDFRGRVYAQGSLISPQGGDLQKGLIRFAEAYPLGEKGAYWFKVQGANVWGWDKEIFDERVSRCETEEFKDMCLDIAADPISFTDWTKADKPWQFLAWCFEYAALLEWIDEGNDESDFLSHVAVAMDGSCSGIQHYSAMLRDSVGGKEVNLLPSDKPQDIYGAVANIVRGWMESVIDDYTQDVPMWDKIEEKFGAVKGYKFAEEWLRVCVTRSMTKKPVMTLPYGSSQLTCRDSMDDYLSDLQSKADKKALATGMATGNIHNFTDKDGDLPRNEAVSYASMMTWKAIGEVVIAARAAMKYIKEVTKVVAELGESLEWTTPLGFIVKQSVFEVREDKQVFTNMLGGCKFRVKEITDKIDARRMGSSCAPNFVHSMDASHLLLATTYFSRAGIKAIAVIHDSFGTHAGKTEELRSLLINSFVDMYLEHDVITNFKEENEERLMIEIDVEIPEQGDLDLEEVRLSPYCFA

SEQ ID NO: 10 - protein, 921 aa, Trungvirus VEN
  source 1..921: note = Vibrio phage Ven
MKDLLQVQLEHEAEMHGAGILRFEKNNQRNLDSGNASDADWYRRLTREFVRPMADAIDAYKSYYEGRRGKPSACLSHLRCLSSEKAAYIAMKVIFNSLHGDKIPAQVLANNIGRSIEDEVRFTKLEDVAPAYISAIKDSLKKRSSKSQKFEHDSMVHAEKEVVLFSHFCKLLDNGYTSEKVKELLGINKEKFEHLSQKRERAKEASTNDEYVVDFDRWVAWPVNDVLQLGAKMIDIFANNILLEGQPIIEKVNISSGKGRAKNTPAAIVPTEATEKWIDGYKEVMGNMAPAFEPCVVKPRDWTSPFNGGYHSKEVSSKLHIAKVKNKQHLKRLTQKQMPKVYKALNALQSVRWQIHPSVLDAANQILLRGLPLGMPSTHKPKKPVCPVPAIYADLRGEELMAMLDEEQKEAFFQWKRDTVAYYTNEQKRKADVREAKATLDQANKFSKFEELFFVYTLDFRGRVYAQGSLVSPQGGDLQKALIRFAEAKPLGATGEYWFKVQGANVWGWDKETFAERVSRCETEEFTEMCLDIAADPITFTDWTKADKPWQFLAWCYEYAAYLEWLEDGKDGKDFPSHVAVAMDGSCSGIQHYSAMLRDSVGGKEVNLLPSDKPQDIYGAVSNLVVEWMEAIIDSKESDCPIWDKLEEKYGPVSCYKFAEEWLRLGVNRSMTKRSVMTLPYGSSQLTCRDSIDDYLKDLQSKADKKALASGQAVGTIHRFDKDLPMKDAISFASMLVWRAIGEVVVAAREGMAYIKAMSNAVSSLNKPMEWVTPTGFIVLQEVFTVTDDKQVFTSMMGNCKFRIKEETTTIDKTRMASSSAPNFVHSMDASHLLLSVLAFADNGIDSIAVIHDSFGTHACDTEKLRGLLSGAFVKMYEENDVILQFKEHNEDVLLTVFEVDLPTKGNLDLQEVLVSSYCFA

SEQ ID NO: 11 - protein, 912 aa, Murciavirus CPP1m
  source 1..912: note = Marinomonas phage CPP1m
MTELYSKQLGIEMEMHGAGIARFEKANQRNLEAGQASETEWNRRILRELVKPMSDALEAYIDYYENNAGRMSHSIHYMKCLPPQAASYITIKSVMDNLSGGFIKTRELVMDIAKRIEDEVRFTRLEDTQPKYIKAIKEGLKKRASKSYKFEHDSMVHAERELALLGHFSKLDAAGVDKEEIKRILSLDEDKYKALYSKREYAVDFDRWVAWPDNHLLLLGANMLEIFTENMTLNGNSLIVKENVRDNSKAGVQVLSLIRASDYLNEWVEEYKEAMADMYPTFAPCVIPPRDWESATDGGYHTREVGSKVNLVKIRDKKHLAKLTKEQMPAIYKAVNGLQSVKWAINSEILEIAKEIRSRSLPLGMPSMKKTDPPVCPVPEVLSDLRGEELKRALSTGQWEEFKKWKHSMAAYHTAEVKRSSTIREITSTLDIATKYVEFDSLHFVYTLDFRSRFNAQSTYLTPQGGDLQKSLIRFRKKEKLGKDDGEFWFLVQGANVWGWDKEEFKERVRLAEAEEFQAMCLDIAADPTTFVEWTAAEKPWQFLSWCLEYAKLIEWKEEGNDACEFKSFSIVAMDGSCSGIQHYSGMLRDEVGGKEVNLVPSNKPQDIYRAVSDVALGHMEAIMDFRERDVPNWPAIKDKYGYVKAVELSKGWIETGVTRSLSKKPVMTMPYGSSPRTCYQAVQDYLHDIDAKERKKVLAQGLEYKKMTKFSENLDQKEITSFASSLVWSSIGEVVVAARAGMKFIKQVTGKVSASNKPLVWTTPTGFIVKQAIYVVDSNNRVNTKLMGGTSFRDYRETKQINATKMASSAAPNFIHSMDASHLCFAVNGFIDAGIADIAVIHDSFGTHACHTKKARSILNNTFVDMYQENDVIAGFKEAAEGQIKDKIDVELPEYGTLDLEVVRDSEYLFG

SEQ ID NO: 12 - protein, 912 aa, Murciavirus CB5A
  source 1..912: note = Marinomonas phage CB5A
MTELYSKQLEIEMEMHGAGIARFEKANQRNLEAGQASETEWNRRILRELVKPMSDALEAYIDYYENNAGRMSHSIHYMKCLPPQAASYITIKSVMDHLSGGFIKTRELVMDIAKRIEDEVRFTRLEDTQPKYIKAIKEGLKKRASRSYKFEHDSMVHAERELALLGHFSKLDAAGVDKEEIKRILSLDEDKYKVLYSKREYAVDFDRWVAWPDNHLLLLGANMLEIFTENMTLNGNPLIVKENVRDNSKAGVQVLSLIRASDYLNEWVEEYKEAMADMYPTFAPCVIPPRDWESATDGGYHTREVGSKVNLVKIRDKKHLAKLTKEQMPAIYKAVNGLQSVKWAINSEILEIAKEIRSRSLPLGMPSMKKTDPPVCPVPEVLSDLRGEELKRALSTGQWEEFKKWKHSMAAYHTAEVKRSSTIREITSTLDIATKYVEFDSLHFVYTLDFRSRFNAQSTYLTPQGGDLQKSLIRFRKKEKLGKDDGEFWFLVQGANVWGWDKEEFKERVRLAEAEEFQAMCLDIAADPTTFVEWTAAEKPWQFLSWCLEYAKLIEWKEEGNDACEFESFSIVAMDGSCSGIQHYSGMLRDEVGGKEVNLVPSNKPQDIYRAVSDVALGHMEAILDFRERDVPNWPAIKDKYGYVKAVELSKGWIETGVTRSLSKTPVMTMPYGSSPRTCYQAVQDYLHDIDAKERKKVLAQGLEYKKMTKFSENLDQKEITSFASGLVWSSIGEVVVAARAGMKFIKQVTGKVSASNKPLVWTTPTGFIVKQAIYVVDSDNRVNTKLMGGTSFRDYRETKQINATKMASSAAPNFIHSMDASHLCFAVNGFIDAGIADIAVIHDSFGTHACHTKKARSILNNTFVDMYQENDVIAGFKEAAEGQIKDKIDVELPEYGTLDLEVVRDSEYLFG

SEQ ID NO: 13 - protein, 895 aa, synthetic construct
MLETQLILESKMHGAGMERFVRNNDRAINAGAASETDWFRRLTRNWVKPMADGIQAYLDYYEGRRGRPSASLVHLRCLPTEVASYIAIKTIFDGLRDPSMTAQNLAERIGRRIEDEVRFTKLETAAPKYIKAIKESLKRKASQKYSHGHDVLVHAEKLLSEDQSRKDKFGVEMTRFEGWAENDIFNLGSKLIELFANNMLLNGVPVIRKRNIHRSLRDEIVIIEATEAMEAWIEEYKVVIGEMSPSYAPCVVRPLDWTGPRMGGWHTEKMRRGMPLVKCNDHKVLSRLTKAQMPSVYAAVNALQGVEWRVNKRILQVANEARLRGLPLAIPARERFEMPPCPVPSHFSALRGAELMDALEPVQQEAFRAWKMEAAKTHNDEAERKAKYRSVSATIDQGIQYQDFERLHFVYTCDFRGRVYAQSSLISPQGGDLQKALCQFANGMELGDSGEYWFKVHGANVWGWDKKEFDERVSLVSEQSFIDDCLDIAADPLTFTDWTQADSPWQFLAWCFEYADFVSHCETSPAEKFISRVAVAMDGSCSGIQHYSAMLRDEIGGKEVNLVPSDRPQDIYGAVAKIVARWMEDIVEGKVPCESFERMLTKGEKLLSNSEWSAAQQAPFVLNAFESQMIAAEWLRIGVTRSLCKKPVMTLPYGSSQMTCREAMSQHLEQLQKDENEKARGQFRKPAMMHGFNDGKGSMSRQQAELFASGLTWEAIGDVVVAARAAMTFIKKVTHEVSQRNTALEWATPTGFIVHQSIFDTTCRRVKTQMMGDTFFTLREETPVISVNKMKSSAAPNFVHSMDASHLVLAVEGFSNNAIHSIAVVHDSFGTHAGNTAKLRSILSNSFVDMYQKHDVVKDFYDQQCDRVMCDLELDLPATGSLDLEAVRTSKYMFA

SEQ ID NO: 14 - protein, 895 aa, synthetic construct
MLDTQLTLEAKMHGAGMERFVRNNERAISGGAASDTDWFRRLTRNFVKPMAEGIQAYLDYYEGRRGRPSASLVHLRCMPTEVSSYIAIKTIFDGLMNPNQTAQNLAEQIGRRIEDEVRFTKLENAAPKYIKAIKESLKRNASQKYSHGHDVLVHAEKLLSEDQARKDKYGVDLERFTGWSDNDIFNLGGKLIELFASNMLLNGVPVIRKRNIHRSLRDEVVVIEATEAMEQWIEEYKVVIGEMSPAYAPCVVRPLDWTGPRMGGWHTEKMRRGMPLVKSRDHKVLSRLTKAQMPAVYAAVNALQGVEWRVNQRILQVANEARLRGLPLAMAAREKVETPPCPVPSHFADLRGSELMEVLEPEQQEAFKKWKMEAAKAYNAEAERKAKYREDSATIDQGIQYQEFERMHFVYTCDFRGRVYAQSSLISPQGGDLQKALCQFANGMALGDTGEYWFKVHGANVWGWDKKEFDERVQLVSEADFIDRCLDIAADPLTFTEWTQADSPWQFLAWCFEYADFVSHCETSPAEKFISRVAVAMDGSCSGIQHYSAMLRDEIGGKEVNLVPSDRPQDIYGAVAKIVARWMEDIVEGKVPCESFERMLAKGERILTTGDWTAAHQAPFVLNAFESQMIAAEWLRIGVTRSLCKKPVMTLPYGSSQMTCRESMGQHLDAMQKEENEKARGQFRKPVAMHNFTDGKGSMSRQQAELFASGLTWEGIGHVVVAARAAMTFIKKVTHEVGSRNHALEWVTPTGFIVRQSIFDSTCRRVKTQMMGETFFTIREETPVIAVAKMKASAPPNFVHSMDASHLVLATEESSNNAIHSLAVVHDSFGTHAGNTAKLRSILANTFVDMYLQHDVIKDFYDAQCDAVMVDLELDLPATGTLDLEAVRTSKYLFA

SEQ ID NO: 15 - protein, 895 aa, synthetic construct
MLDTQLTLEAKMHGAGMERFVRNNERAISGGAASDTDWFRRLTRNFVKPMAEGIQAYLDYYEGRRGRPSASLVHLRCMPTEVSSYIAIKTIFDGLMNPNQTAQNLAEQIGRRIEDEVRFTKLENAAPKYIKAIKESLKRNASQKYSHGHDVLVHAEKLLSEDQARKDKYGVDLERFTGWSDNDIFNLGGKLIELFASNMLLNGVPVIRKRNIHRSLRDEVVVIEATEAMEQWIEEYKVVIGEMSPAYAPCVVRPLDWTGPRMGGWHTEKMRRGMPLVKSRDHKVLSRLTKAQMPAVYAAVNALQGVEWRVNKRILQVANEARLRGLPLAMAAREKVETPPCPVPSHFADLRGSELMEVLEPEQQEAFKKWKMEAAKAYNAEAERKAKYREDAATIDQGIQYQEFERMHFVYTCDFRGRVYAQSSLISPQGGDLQKALCQFANGMALGDTGEYWFKVHGANVWGWDKKEFDERVQLVSEADFIDRCLDIAADPLTFTEWTQADSPWQFLAWCFEYADFVSHCETSPAEKFISRVAVAMDGSCSGIQHYSAMLRDEIGGKEVNLVPSDRPQDIYGAVAKIVARWMEDIVEGKVPCESFERMLAKGERILTTGDWTAAHQAPFVLNAFESQMIAAEWLRIGVTRSLCKKPVMTLPYGSSQMTCRESMGQHLDAMQKEENEKARGQFRKPVAMHNFTDGKGSMSRQQAELFASGLTWEGIGHVVVAARAAMTFIKKVTHEVGSRNHALEWVTPTGFIVRQSIFDSTCRRVKTQMMGETFFTIREETPVIAVAKMKASAPPNFVHSMDASHLVLATEESSNNAIHSLAVVHDSFGTHAGNTAKLRSILANTFVDMYLQHDVIKDFYDAQCDAVMVDLELDLPATGTLDLEAVRTSKYLFA

SEQ ID NO: 16 - protein, 895 aa, synthetic construct
MGSSHHHHHHPNIETQEALEAKMHGAGLDRFIRNNDRAITGGSASDTDWYRRLTRQFVKPMADGIQAYLDYYEGRRGKPSASLVHLRCLPTETASYIAIKTIFDGLTNPNQTAQNLSEQIGKRIEDEVRFMKLDNAAPKFIKAIKESLKRSSSQKYSHGHDVMVHAEKLLAEDKERQDKYGVMIDRHLSWPDNDIFNLGSKLIELFANNMLLDGEVLIQKRLIVRSQIDRVTVIEATPAMEKWIEEYKVVVGEMAPAFAPCVIKPLDWTGPRFGGWHTEKMRRQMPLAKCKQQSVLSRLTKKQMPRVYSAVNALQAVEWRVNHRILSVANEARLRGLPLALPDREKLATPPCPVPHMYAELRGAELMSVLSESQKEAFRTWKADAAKTYNDEAERKAKYREVCATIDQGIQYKDFDKIHFVYTLDFRGRVYCQSSLISPQGGDLQKSLCQFANGMPLGESGVHWFKVQGANVWGWDKKQFEERDELVMADDFVELCLDIAADPLTFTDWTQADKPWQFLAWCFEYADFVSHCETNPPETFVSRIAVAMDGSCSGIQHYSAMLRDEIGGKEVNLVPGDRPQDIYGAVAKIVGRWMEDISELQVPCESFTRMLSKGAVEGQFKLCPLEAQMLSRAWVEIGVTRSMTKKPVMTLPYGSSQMTCRESMAQYLNDLQKKENEQSRAQNRKPVAVHPFNDSKGSMSRMQAELFASGLTWEAIGHVVVAARAAMRFIKKVTHETSSRNVALEWIAPTGFIVNQCIFDSTPRRVKTQMMGETFFTIREENPTISVNKMKSSAAPNFVHSMDASHLCIAVDEFALAGITSIAVIHDSFGTHAGNTERMRKLLINSFVDMYQNHDVIRDFYEAQCERVKCDLELDLPAVGTLDLETVRHSKYIFA

SEQ ID NO: 17 - protein, 952 aa, synthetic construct
  source 1..952: note = B=D or N, J =I or L, Z = Q or X= any residue
MXXXXXXXQXXXEXXMHGAGXXRFXXXNXRXXXXGXXXXXXWXRRJXRXXVXPMXXXJXAYXXYYXXXXGXXSXXJXXXXCXXXZXXXYIXXKXXXBXXXXXXXXXXXJXXXIXXXIEDEXRFXXLXXXXPXXIXAIKXXLKXXXXXXXXXXHDXXVHAEXXXXXXXXXXXXXXXXXXXXXXXXJLXXBXXXXXXXXXXXXXXXXXXXXXXXXXXXXRXXXWXXNXXXXLGXXXXXJFXXNXXXXGXXXXXXXXXXXXXXXXXXXXXXJXXXXXXXXWXXXYKXXXXXXXPXXXPCVXXPXXWXXXXXGGXHXXXXXXXXXXXKXXXXXXLXXLXXXQMPXXYXAXNXLQXVXWXXXXXXXXXAXZXXXRXLPXXXXXXXXXXXPXCPVPXXXXXLRGXXLXXXLXXXQXXXFXXWKXXXXXXXXXEXXRXXXXRXXXXTJDXXXXXXXXXXXXXVYTXDFRXRXXXXXXXXXPQGGDLQKXLXXFXXXXXXGXXXGXXWFXXXGANVWGWDKXXFXERXXXXXXXXFXXXXXCLDIAADPXXFXXWTXAXXPWQFLXWCXEYAXXXXXXXXXXXXXXFXSXXXVAMDGSCSGIQHYSXMLRDXXGGXEVNLXPXXXPXDIYXAVXXXXXXXMEXXXXXXXXXXXXXXXXXXXXXXXXXXXXXXXXXXXXXXXXXXXXXXXXXXXXXXXWXXXXVXRSXXKXXVMTXPYGSSXXTCXXXXXXXLXXXXXXXXXXXXXXXXXXXXXXXFXXXXXXXXXXXXXXXASXXXWXXIGXVVXAARXXMXXIKXXXXXXXXXXXXXXXXXPXGFIVXQXXXXXXXXXXXXTXXXGXXXFXXXXXXXXXXXXXMXXSXXPNFXHSMDASHLXXXXXXXXXXXIXXJAVXHDSFGTHAXXTXXXRXXLXXXFVXMYXXXDVXXXFXXXXXXXXXXXXXXXXPXXGXLDLZXVXXSXYXFX

SEQ ID NO: 18 - protein, 880 aa, synthetic construct
MEDLMEVQLELEAEMHGAGIERFEKNNQRAIDSGSASDTDWYRRLTREFVKPMADGIQAYLDYYEGRRGRPSASLTHLKCLPPEAAAYIAIKTIFDSLSNHNITAQNLANNIGRRIEDEVRFTKLEDAAPKYIKAIKESLKRRSSQSYKHEHDVMVHAEKKLAEDKERKDEYAVDIDRWVSWPDNDVLHLGSKLIEIFANNMLLDGEPVIQKQNISTGGRKTVAVIAPTEALEEWIEEYKEVMGEMSPAYAPCVVPPRDWTSPTNGGYHTEEVSSKLPLVKCRDKKHLRRLTRKQMPKVYKAVNALQAVKWQINERILAVANEVRLRGLPLGMPSREKLDKPPCPVPEMFSDLRGEELMAALDPDQWEAFKEWKREAAKTYNDEQERKSKFREVVATLDQANKYKDFDSIHFVYTLDFRGRVYCQSSLVSPQGGDLQKALIRFADGMPLGEEGEYWFKVHGANVWGWDKKPFDERVSLVSEEEFTEMCLDIAADPITFTDWTKADKPWQFLAWCFEYADLLEWIEEGNNPEDFVSHIAVAMDGSCSGIQHYSAMLRDSVGGKEVNLVPSDKPQDIYGAVAKVVVKWMEAIVDGNERDANFEKLEEKLNPVKAQKLAKEWLRIGVTRSLTKKPVMTLPYGSSQLTCRESVSDYLKDLQKKENKKARAQGREPKKVHPFNDKDGSMPLKEAESFASGLTWESIGEVVVAARAGMKFIKEVTNEVAKANKPLEWTTPTGFIVKQAIYETESRRVKTQMMGGTFFTIREETDKIDVRKMKSSAAPNFVHSMDASHLVLAVNGFADNGITSIAVIHDSFGTHAGNTEKLRSLLSNSFVDMYQENDVITDFKEHNEDRLMTEIEVEVPEKGDLDLEEVRESEYCFA

SEQ ID NO: 19 - protein, 884 aa, synthetic construct
MKDLMQLELEAKMHGAGIERFERNNQRAIEGGAASDTDWFRRLTREFVKPMADGIQAYLDYYEGRRGRPSASLVHLRCLPTEVASYIAIKTIFDGLMNPNQTAQNLAEQIGRRIEDEVRFTKLENAAPKYIKAIKESLKRRASQKYSHGHDVMVHAEKLLSEDQDRKDKYGVDLDRWIGWSDNDIFNLGAKLIELFANNMLLNGVPVIRKRNIHRSLRDEVVVIEATEAMEQWIEEYKVVVGEMSPAYAPCVVRPLDWTGPRNGGWHTEKMRRGMPLVKCRDKKVLSRLTKKQMPKVYAAVNALQGVEWRVNPRILQVANEARLRGLPLGMPAREKLETPPCPVPSHFSDLRGEELMAVLDPEQKEAFKKWKMEAAKTYNDEAERKAKYREVVATIDQGIQYKDFERLHFVYTLDFRGRVYAQSSLISPQGGDLQKALCQFANGMPLGDTGEYWFKVHGANVWGWDKEEFDERVSLVETEEFIDMCLDIAADPLTFTDWTQADKPWQFLAWCFEYADFVSHCETNPAEDFPSRVAVAMDGSCSGIQHYSAMLRDEVGGKEVNLVPSDKPQDIYGAVAKIVARWMEDIVEGEVPCESFERMLAKGLTAPFKLNAVEAQMIAAEWLRIGVTRSLTKKPVMTLPYGSSQMTCRESMAQYLDDLQKKENEKARAQFRKPVAMHNFTDGKGSMSRQQAELFASGLTWEAIGEVVVAARAAMKFIKKVTHEVSSRNKPLEWVTPTGFIVKQAIYDSTDRRVKTQMMGETFFTIREETPVISVRKMKSSAAPNFVHSMDASHLVLAVEGFSDNGITSIAVIHDSFGTHAGNTEKLRSILSNSFVDMYQEHDVIKDFYEAQCDRVMTDLELDLPATGTLDLEEVRTSKYIFA

SEQ ID NO: 20 - protein, 1765 aa, synthetic construct
MAKRLQRCQDVNQVCEIYNSKGGIGELELRFDKLPQNLFAGVFDKLKPDGEIQTTMRVSNRDGVAREITFGGGVKTNEIFVKKQNICVFDVVDIFSYKVAVSTEETVVEKPTMETTAGVRFKIRLSVEDVVKDWRIDLTAVKTAELGKIAQHTASIVQRTFPDNLLKLTGAEVAKLAADSYELELEYTGKSPATNEKVNVAAKYAVELLSSVRNANSTAAASFGESVSDLCRVAKIIHTHEYANVVCRTPSFKMLLPQVVSLTKSSYYGGLYPPENLWLAGKTDGVRALVVCEDGVAKVITAESVDITHGVCSATTILDCELNVDAKILYVFDVIISNNTQVYTQPFSTRITTDISDIKIDGYKIEMKPFVKVVKADEATFKSAYKAPHNEGLIMIEDGAAYAATKTYKWKPLSHNTIDFLIKACPKQLINVDPYKPRAGYKLWLLFTTISLDQQRELGIEFIPAWKILFTDINMFGSRVPIQFQPAINPLAYVCYLPEDVNVNDGDIVEMRAVDGYDTIPKWELVRSRNDRKNEPGFYGNNYKIASDIYLNYIDVFHFEDLYKYNPGYFEKNKSDIYVAPNKYRRYLIKSLFGRYLRDAKWVIDAAAGRGADLHLYKAECVEHLLAIDIDPTAISELVRRRNEITGYNKSHRGGRNMHSHRGQSHCAKSTSLHALVADLRENPDVLIPKIIQSRPHERCYDAIVINFAIHYLCDTDEHIRDFLITVSRLLAPNGVFIFTTMDGESIVKLLADHKVRPGEAWTIHTGDVNSPDSTVPKYSIRRLYDSDKLTKTGQQIEVLLPMSGEMKAEPLCNIKNIISMARKMGLDLVESANFSVLYEAYARDYPDIYARMTPDDKLYNDLHTYAVFKRKKGAATAGTPNIETQEALEAKMHGAGLDRFIRNNDRAITGGSASDTDWYRRLTRQFVKPMADGIQAYLDYYEGRRGKPSASLVHLRCLPTETASYIAIKTIFDGLTNPNQTAQNLSEQIGKRIEDEVRFMKLDNAAPKFIKAIKESLKRSSSQKYSHGHDVMVHAEKLLAEDKERQDKYGVMIDRHLSWPDNDIFNLGSKLIELFANNMLLDGEVLIQKRLIVRSQIDRVTVIEATPAMEKWIEEYKVVVGEMAPAFAPCVIKPLDWTGPRFGGWHTEKMRRQMPLAKCKQQSVLSRLTKKQMPRVYSAVNALQAVEWRVNHRILSVANEARLRGLPLALPDREKLATPPCPVPHMYAELRGAELMSVLSESQKEAFRTWKADAAKTYNDEAERKAKYREVCATIDQGIQYKDFDKIHFVYTLDFRGRVYCQSSLISPQGGDLQKSLCQFANGMPLGESGVHWFKVQGANVWGWDKKQFEERDELVMADDFVELCLDIAADPLTFTDWTQADKPWQFLAWCFEYADFVSHCETNPPETFVSRIAVAMDGSCSGIQHYSAMLRDEIGGKEVNLVPGDRPQDIYGAVAKIVGRWMEDISELQVPCESFTRMLSKGAVEGQFKLCPLEAQMLSRAWVEIGVTRSMTKKPVMTLPYGSSQMTCRESMAQYLNDLQKKENEQSRAQNRKPVAVHPFNDSKGSMSRMQAELFASGLTWEAIGHVVVAARAAMRFIKKVTHETSSRNVALEWIAPTGFIVNQCIFDSTPRRVKTQMMGETFFTIREENPTISVNKMKSSAAPNFVHSMDASHLCIAVDEFALAGITSIAVIHDSFGTHAGNTERMRKLLINSFVDMYQNHDVIRDFYEAQCERVKCDLELDLPAVGTLDLETVRHSKYIFA

SEQ ID NO: 21 - protein, 1764 aa, synthetic construct
MAKRLQRCQDVNQVCEIYNSKGGIGELELRFDKLPQNLFAGVFDKLKPDGEIQTTMRVSNRDGVAREITFGGGVKTNEIFVKKQNICVFDVVDIFSYKVAVSTEETVVEKPTMETTAGVRFKIRLSVEDVVKDWRIDLTAVKTAELGKIAQHTASIVQRTFPDNLLKLTGAEVAKLAADSYELELEYTGKSPATNEKVNVAAKYAVELLSSVRNANSTAAASFGESVSDLCRVAKIIHTHEYANVVCRTPSFKMLLPQVVSLTKSSYYGGLYPPENLWLAGKTDGVRALVVCEDGVAKVITAESVDITHGVCSATTILDCELNVDAKILYVFDVIISNNTQVYTQPFSTRITTDISDIKIDGYKIEMKPFVKVVKADEATFKSAYKAPHNEGLIMIEDGAAYAATKTYKWKPLSHNTIDFLIKACPKQLINVDPYKPRAGYKLWLLFTTISLDQQRELGIEFIPAWKILFTDINMFGSRVPIQFQPAINPLAYVCYLPEDVNVNDGDIVEMRAVDGYDTIPKWELVRSRNDRKNEPGFYGNNYKIASDIYLNYIDVFHFEDLYKYNPGYFEKNKSDIYVAPNKYRRYLIKSLFGRYLRDAKWVIDAAAGRGADLHLYKAECVEHLLAIDIDPTAISELVRRRNEITGYNKSHRGGRNMHSHRGQSHCAKSTSLHALVADLRENPDVLIPKIIQSRPHERCYDAIVINFAIHYLCDTDEHIRDFLITVSRLLAPNGVFIFTTMDGESIVKLLADHKVRPGEAWTIHTGDVNSPDSTVPKYSIRRLYDSDKLTKTGQQIEVLLPMSGEMKAEPLCNIKNIISMARKMGLDLVESANFSVLYEAYARDYPDIYARMTPDDKLYNDLHTYAVFKRKKGAATAGPNIETQEALEAKMHGAGLDRFIRNNDRAITGGSASDTDWYRRLTRQFVKPMADGIQAYLDYYEGRRGKPSASLVHLRCLPTETASYIAIKTIFDGLTNPNQTAQNLSEQIGKRIEDEVRFMKLDNAAPKFIKAIKESLKRSSSQKYSHGHDVMVHAEKLLAEDKERQDKYGVMIDRHLSWPDNDIFNLGSKLIELFANNMLLDGEVLIQKRLIVRSQIDRVTVIEATPAMEKWIEEYKVVVGEMAPAFAPCVIKPLDWTGPRFGGWHTEKMRRQMPLAKCKQQSVLSRLTKKQMPRVYSAVNALQAVEWRVNHRILSVANEARLRGLPLALPDREKLATPPCPVPHMYAELRGAELMSVLSESQKEAFRTWKADAAKTYNDEAERKAKYREVCATIDQGIQYKDFDKIHFVYTLDFRGRVYCQSSLISPQGGDLQKSLCQFANGMPLGESGVHWFKVQGANVWGWDKKQFEERDELVMADDFVELCLDIAADPLTFTDWTQADKPWQFLAWCFEYADFVSHCETNPPETFVSRIAVAMDGSCSGIQHYSAMLRDEIGGKEVNLVPGDRPQDIYGAVAKIVGRWMEDISELQVPCESFTRMLSKGAVEGQFKLCPLEAQMLSRAWVEIGVTRSMTKKPVMTLPYGSSQMTCRESMAQYLNDLQKKENEQSRAQNRKPVAVHPFNDSKGSMSRMQAELFASGLTWEAIGHVVVAARAAMRFIKKVTHETSSRNVALEWIAPTGFIVNQCIFDSTPRRVKTQMMGETFFTIREENPTISVNKMKSSAAPNFVHSMDASHLCIAVDEFALAGITSIAVIHDSFGTHAGNTERMRKLLINSFVDMYQNHDVIRDFYEAQCERVKCDLELDLPAVGTLDLETVRHSKYIFA

SEQ ID NO: 22 - protein, 1772 aa, synthetic construct
MAKRLQRCQDVNQVCEIYNSKGGIGELELRFDKLPQNLFAGVFDKLKPDGEIQTTMRVSNRDGVAREITFGGGVKTNEIFVKKQNICVFDVVDIFSYKVAVSTEETVVEKPTMETTAGVRFKIRLSVEDVVKDWRIDLTAVKTAELGKIAQHTASIVQRTFPDNLLKLTGAEVAKLAADSYELELEYTGKSPATNEKVNVAAKYAVELLSSVRNANSTAAASFGESVSDLCRVAKIIHTHEYANVVCRTPSFKMLLPQVVSLTKSSYYGGLYPPENLWLAGKTDGVRALVVCEDGVAKVITAESVDITHGVCSATTILDCELNVDAKILYVFDVIISNNTQVYTQPFSTRITTDISDIKIDGYKIEMKPFVKVVKADEATFKSAYKAPHNEGLIMIEDGAAYAATKTYKWKPLSHNTIDFLIKACPKQLINVDPYKPRAGYKLWLLFTTISLDQQRELGIEFIPAWKILFTDINMFGSRVPIQFQPAINPLAYVCYLPEDVNVNDGDIVEMRAVDGYDTIPKWELVRSRNDRKNEPGFYGNNYKIASDIYLNYIDVFHFEDLYKYNPGYFEKNKSDIYVAPNKYRRYLIKSLFGRYLRDAKWVIDAAAGRGADLHLYKAECVEHLLAIDIDPTAISELVRRRNEITGYNKSHRGGRNMHSHRGQSHCAKSTSLHALVADLRENPDVLIPKIIQSRPHERCYDAIVINFAIHYLCDTDEHIRDFLITVSRLLAPNGVFIFTTMDGESIVKLLADHKVRPGEAWTIHTGDVNSPDSTVPKYSIRRLYDSDKLTKTGQQIEVLLPMSGEMKAEPLCNIKNIISMARKMGLDLVESANFSVLYEAYARDYPDIYARMTPDDKLYNDLHTYAVFKRKKGAATAGTGAATAGTPNIETQEALEAKMHGAGLDRFIRNNDRAITGGSASDTDWYRRLTRQFVKPMADGIQAYLDYYEGRRGKPSASLVHLRCLPTETASYIAIKTIFDGLTNPNQTAQNLSEQIGKRIEDEVRFMKLDNAAPKFIKAIKESLKRSSSQKYSHGHDVMVHAEKLLAEDKERQDKYGVMIDRHLSWPDNDIFNLGSKLIELFANNMLLDGEVLIQKRLIVRSQIDRVTVIEATPAMEKWIEEYKVVVGEMAPAFAPCVIKPLDWTGPRFGGWHTEKMRRQMPLAKCKQQSVLSRLTKKQMPRVYSAVNALQAVEWRVNHRILSVANEARLRGLPLALPDREKLATPPCPVPHMYAELRGAELMSVLSESQKEAFRTWKADAAKTYNDEAERKAKYREVCATIDQGIQYKDFDKIHFVYTLDFRGRVYCQSSLISPQGGDLQKSLCQFANGMPLGESGVHWFKVQGANVWGWDKKQFEERDELVMADDFVELCLDIAADPLTFTDWTQADKPWQFLAWCFEYADFVSHCETNPPETFVSRIAVAMDGSCSGIQHYSAMLRDEIGGKEVNLVPGDRPQDIYGAVAKIVGRWMEDISELQVPCESFTRMLSKGAVEGQFKLCPLEAQMLSRAWVEIGVTRSMTKKPVMTLPYGSSQMTCRESMAQYLNDLQKKENEQSRAQNRKPVAVHPFNDSKGSMSRMQAELFASGLTWEAIGHVVVAARAAMRFIKKVTHETSSRNVALEWIAPTGFIVNQCIFDSTPRRVKTQMMGETFFTIREENPTISVNKMKSSAAPNFVHSMDASHLCIAVDEFALAGITSIAVIHDSFGTHAGNTERMRKLLINSFVDMYQNHDVIRDFYEAQCERVKCDLELDLPAVGTLDLETVRHSKYIFA

SEQ ID NO: 23 - protein, 1770 aa, synthetic construct
MAKRLQRCQDVNQVCEIYNSKGGIGELELRFDKLPQNLFAGVFDKLKPDGEIQTTMRVSNRDGVAREITFGGGVKTNEIFVKKQNICVFDVVDIFSYKVAVSTEETVVEKPTMETTAGVRFKIRLSVEDVVKDWRIDLTAVKTAELGKIAQHTASIVQRTFPDNLLKLTGAEVAKLAADSYELELEYTGKSPATNEKVNVAAKYAVELLSSVRNANSTAAASFGESVSDLCRVAKIIHTHEYANVVCRTPSFKMLLPQVVSLTKSSYYGGLYPPENLWLAGKTDGVRALVVCEDGVAKVITAESVDITHGVCSATTILDCELNVDAKILYVFDVIISNNTQVYTQPFSTRITTDISDIKIDGYKIEMKPFVKVVKADEATFKSAYKAPHNEGLIMIEDGAAYAATKTYKWKPLSHNTIDFLIKACPKQLINVDPYKPRAGYKLWLLFTTISLDQQRELGIEFIPAWKILFTDINMFGSRVPIQFQPAINPLAYVCYLPEDVNVNDGDIVEMRAVDGYDTIPKWELVRSRNDRKNEPGFYGNNYKIASDIYLNYIDVFHFEDLYKYNPGYFEKNKSDIYVAPNKYRRYLIKSLFGRYLRDAKWVIDAAAGRGADLHLYKAECVEHLLAIDIDPTAISELVRRRNEITGYNKSHRGGRNMHSHRGQSHCAKSTSLHALVADLRENPDVLIPKIIQSRPHERCYDAIVINFAIHYLCDTDEHIRDFLITVSRLLAPNGVFIFTTMDGESIVKLLADHKVRPGEAWTIHTGDVNSPDSTVPKYSIRRLYDSDKLTKTGQQIEVLLPMSGEMKAEPLCNIKNIISMARKMGLDLVESANFSVLYEAYARDYPDIYARMTPDDKLYNDLHTYAVFKRKKGAATAGTGAATAPNIETQEALEAKMHGAGLDRFIRNNDRAITGGSASDTDWYRRLTRQFVKPMADGIQAYLDYYEGRRGKPSASLVHLRCLPTETASYIAIKTIFDGLTNPNQTAQNLSEQIGKRIEDEVRFMKLDNAAPKFIKAIKESLKRSSSQKYSHGHDVMVHAEKLLAEDKERQDKYGVMIDRHLSWPDNDIFNLGSKLIELFANNMLLDGEVLIQKRLIVRSQIDRVTVIEATPAMEKWIEEYKVVVGEMAPAFAPCVIKPLDWTGPRFGGWHTEKMRRQMPLAKCKQQSVLSRLTKKQMPRVYSAVNALQAVEWRVNHRILSVANEARLRGLPLALPDREKLATPPCPVPHMYAELRGAELMSVLSESQKEAFRTWKADAAKTYNDEAERKAKYREVCATIDQGIQYKDFDKIHFVYTLDFRGRVYCQSSLISPQGGDLQKSLCQFANGMPLGESGVHWFKVQGANVWGWDKKQFEERDELVMADDFVELCLDIAADPLTFTDWTQADKPWQFLAWCFEYADFVSHCETNPPETFVSRIAVAMDGSCSGIQHYSAMLRDEIGGKEVNLVPGDRPQDIYGAVAKIVGRWMEDISELQVPCESFTRMLSKGAVEGQFKLCPLEAQMLSRAWVEIGVTRSMTKKPVMTLPYGSSQMTCRESMAQYLNDLQKKENEQSRAQNRKPVAVHPFNDSKGSMSRMQAELFASGLTWEAIGHVVVAARAAMRFIKKVTHETSSRNVALEWIAPTGFIVNQCIFDSTPRRVKTQMMGETFFTIREENPTISVNKMKSSAAPNFVHSMDASHLCIAVDEFALAGITSIAVIHDSFGTHAGNTERMRKLLINSFVDMYQNHDVIRDFYEAQCERVKCDLELDLPAVGTLDLETVRHSKYIFA

SEQ ID NO: 24 - protein, 1768 aa, synthetic construct
MAKRLQRCQDVNQVCEIYNSKGGIGELELRFDKLPQNLFAGVFDKLKPDGEIQTTMRVSNRDGVAREITFGGGVKTNEIFVKKQNICVFDVVDIFSYKVAVSTEETVVEKPTMETTAGVRFKIRLSVEDVVKDWRIDLTAVKTAELGKIAQHTASIVQRTFPDNLLKLTGAEVAKLAADSYELELEYTGKSPATNEKVNVAAKYAVELLSSVRNANSTAAASFGESVSDLCRVAKIIHTHEYANVVCRTPSFKMLLPQVVSLTKSSYYGGLYPPENLWLAGKTDGVRALVVCEDGVAKVITAESVDITHGVCSATTILDCELNVDAKILYVFDVIISNNTQVYTQPFSTRITTDISDIKIDGYKIEMKPFVKVVKADEATFKSAYKAPHNEGLIMIEDGAAYAATKTYKWKPLSHNTIDFLIKACPKQLINVDPYKPRAGYKLWLLFTTISLDQQRELGIEFIPAWKILFTDINMFGSRVPIQFQPAINPLAYVCYLPEDVNVNDGDIVEMRAVDGYDTIPKWELVRSRNDRKNEPGFYGNNYKIASDIYLNYIDVFHFEDLYKYNPGYFEKNKSDIYVAPNKYRRYLIKSLFGRYLRDAKWVIDAAAGRGADLHLYKAECVEHLLAIDIDPTAISELVRRRNEITGYNKSHRGGRNMHSHRGQSHCAKSTSLHALVADLRENPDVLIPKIIQSRPHERCYDAIVINFAIHYLCDTDEHIRDFLITVSRLLAPNGVFIFTTMDGESIVKLLADHKVRPGEAWTIHTGDVNSPDSTVPKYSIRRLYDSDKLTKTGQQIEVLLPMSGEMKAEPLCNIKNIISMARKMGLDLVESANFSVLYEAYARDYPDIYARMTPDDKLYNDLHTYAVFKRKKGAATAGTGAAPNIETQEALEAKMHGAGLDRFIRNNDRAITGGSASDTDWYRRLTRQFVKPMADGIQAYLDYYEGRRGKPSASLVHLRCLPTETASYIAIKTIFDGLTNPNQTAQNLSEQIGKRIEDEVRFMKLDNAAPKFIKAIKESLKRSSSQKYSHGHDVMVHAEKLLAEDKERQDKYGVMIDRHLSWPDNDIFNLGSKLIELFANNMLLDGEVLIQKRLIVRSQIDRVTVIEATPAMEKWIEEYKVVVGEMAPAFAPCVIKPLDWTGPRFGGWHTEKMRRQMPLAKCKQQSVLSRLTKKQMPRVYSAVNALQAVEWRVNHRILSVANEARLRGLPLALPDREKLATPPCPVPHMYAELRGAELMSVLSESQKEAFRTWKADAAKTYNDEAERKAKYREVCATIDQGIQYKDFDKIHFVYTLDFRGRVYCQSSLISPQGGDLQKSLCQFANGMPLGESGVHWFKVQGANVWGWDKKQFEERDELVMADDFVELCLDIAADPLTFTDWTQADKPWQFLAWCFEYADFVSHCETNPPETFVSRIAVAMDGSCSGIQHYSAMLRDEIGGKEVNLVPGDRPQDIYGAVAKIVGRWMEDISELQVPCESFTRMLSKGAVEGQFKLCPLEAQMLSRAWVEIGVTRSMTKKPVMTLPYGSSQMTCRESMAQYLNDLQKKENEQSRAQNRKPVAVHPFNDSKGSMSRMQAELFASGLTWEAIGHVVVAARAAMRFIKKVTHETSSRNVALEWIAPTGFIVNQCIFDSTPRRVKTQMMGETFFTIREENPTISVNKMKSSAAPNFVHSMDASHLCIAVDEFALAGITSIAVIHDSFGTHAGNTERMRKLLINSFVDMYQNHDVIRDFYEAQCERVKCDLELDLPAVGTLDLETVRHSKYIFA

SEQ ID NO: 25 - protein, 1779 aa, synthetic construct
MAKRLQRCQDVNQVCEIYNSKGGIGELELRFDKLPQNLFAGVFDKLKPDGEIQTTMRVSNRDGVAREITFGGGVKTNEIFVKKQNICVFDVVDIFSYKVAVSTEETVVEKPTMETTAGVRFKIRLSVEDVVKDWRIDLTAVKTAELGKIAQHTASIVQRTFPDNLLKLTGAEVAKLAADSYELELEYTGKSPATNEKVNVAAKYAVELLSSVRNANSTAAASFGESVSDLCRVAKIIHTHEYANVVCRTPSFKMLLPQVVSLTKSSYYGGLYPPENLWLAGKTDGVRALVVCEDGVAKVITAESVDITHGVCSATTILDCELNVDAKILYVFDVIISNNTQVYTQPFSTRITTDISDIKIDGYKIEMKPFVKVVKADEATFKSAYKAPHNEGLIMIEDGAAYAATKTYKWKPLSHNTIDFLIKACPKQLINVDPYKPRAGYKLWLLFTTISLDQQRELGIEFIPAWKILFTDINMFGSRVPIQFQPAINPLAYVCYLPEDVNVNDGDIVEMRAVDGYDTIPKWELVRSRNDRKNEPGFYGNNYKIASDIYLNYIDVFHFEDLYKYNPGYFEKNKSDIYVAPNKYRRYLIKSLFGRYLRDAKWVIDAAAGRGADLHLYKAECVEHLLAIDIDPTAISELVRRRNEITGYNKSHRGGRNMHSHRGQSHCAKSTSLHALVADLRENPDVLIPKIIQSRPHERCYDAIVINFAIHYLCDTDEHIRDFLITVSRLLAPNGVFIFTTMDGESIVKLLADHKVRPGEAWTIHTGDVNSPDSTVPKYSIRRLYDSDKLTKTGQQIEVLLPMSGEMKAEPLCNIKNIISMARKMGLDLVESANFSVLYEAYARDYPDIYARMTPDDKLYNDLHTYAVFKRKKGAATAGTGAATAGTGAATAGTPNIETQEALEAKMHGAGLDRFIRNNDRAITGGSASDTDWYRRLTRQFVKPMADGIQAYLDYYEGRRGKPSASLVHLRCLPTETASYIAIKTIFDGLTNPNQTAQNLSEQIGKRIEDEVRFMKLDNAAPKFIKAIKESLKRSSSQKYSHGHDVMVHAEKLLAEDKERQDKYGVMIDRHLSWPDNDIFNLGSKLIELFANNMLLDGEVLIQKRLIVRSQIDRVTVIEATPAMEKWIEEYKVVVGEMAPAFAPCVIKPLDWTGPRFGGWHTEKMRRQMPLAKCKQQSVLSRLTKKQMPRVYSAVNALQAVEWRVNHRILSVANEARLRGLPLALPDREKLATPPCPVPHMYAELRGAELMSVLSESQKEAFRTWKADAAKTYNDEAERKAKYREVCATIDQGIQYKDFDKIHFVYTLDFRGRVYCQSSLISPQGGDLQKSLCQFANGMPLGESGVHWFKVQGANVWGWDKKQFEERDELVMADDFVELCLDIAADPLTFTDWTQADKPWQFLAWCFEYADFVSHCETNPPETFVSRIAVAMDGSCSGIQHYSAMLRDEIGGKEVNLVPGDRPQDIYGAVAKIVGRWMEDISELQVPCESFTRMLSKGAVEGQFKLCPLEAQMLSRAWVEIGVTRSMTKKPVMTLPYGSSQMTCRESMAQYLNDLQKKENEQSRAQNRKPVAVHPFNDSKGSMSRMQAELFASGLTWEAIGHVVVAARAAMRFIKKVTHETSSRNVALEWIAPTGFIVNQCIFDSTPRRVKTQMMGETFFTIREENPTISVNKMKSSAAPNFVHSMDASHLCIAVDEFALAGITSIAVIHDSFGTHAGNTERMRKLLINSFVDMYQNHDVIRDFYEAQCERVKCDLELDLPAVGTLDLETVRHSKYIFA

SEQ ID NO: 26 - protein, 1781 aa, synthetic construct
MAKRLQRCQDVNQVCEIYNSKGGIGELELRFDKLPQNLFAGVFDKLKPDGEIQTTMRVSNRDGVAREITFGGGVKTNEIFVKKQNICVFDVVDIFSYKVAVSTEETVVEKPTMETTAGVRFKIRLSVEDVVKDWRIDLTAVKTAELGKIAQHTASIVQRTFPDNLLKLTGAEVAKLAADSYELELEYTGKSPATNEKVNVAAKYAVELLSSVRNANSTAAASFGESVSDLCRVAKIIHTHEYANVVCRTPSFKMLLPQVVSLTKSSYYGGLYPPENLWLAGKTDGVRALVVCEDGVAKVITAESVDITHGVCSATTILDCELNVDAKILYVFDVIISNNTQVYTQPFSTRITTDISDIKIDGYKIEMKPFVKVVKADEATFKSAYKAPHNEGLIMIEDGAAYAATKTYKWKPLSHNTIDFLIKACPKQLINVDPYKPRAGYKLWLLFTTISLDQQRELGIEFIPAWKILFTDINMFGSRVPIQFQPAINPLAYVCYLPEDVNVNDGDIVEMRAVDGYDTIPKWELVRSRNDRKNEPGFYGNNYKIASDIYLNYIDVFHFEDLYKYNPGYFEKNKSDIYVAPNKYRRYLIKSLFGRYLRDAKWVIDAAAGRGADLHLYKAECVEHLLAIDIDPTAISELVRRRNEITGYNKSHRGGRNMHSHRGQSHCAKSTSLHALVADLRENPDVLIPKIIQSRPHERCYDAIVINFAIHYLCDTDEHIRDFLITVSRLLAPNGVFIFTTMDGESIVKLLADHKVRPGEAWTIHTGDVNSPDSTVPKYSIRRLYDSDKLTKTGQQIEVLLPMSGEMKAEPLCNIKNIISMARKMGLDLVESANFSVLYEAYARDYPDIYARMTPDDKLYNDLHTYAVFKRKKGAATAGTPGAATAGTPGAATAGTPNIETQEALEAKMHGAGLDRFIRNNDRAITGGSASDTDWYRRLTRQFVKPMADGIQAYLDYYEGRRGKPSASLVHLRCLPTETASYIAIKTIFDGLTNPNQTAQNLSEQIGKRIEDEVRFMKLDNAAPKFIKAIKESLKRSSSQKYSHGHDVMVHAEKLLAEDKERQDKYGVMIDRHLSWPDNDIFNLGSKLIELFANNMLLDGEVLIQKRLIVRSQIDRVTVIEATPAMEKWIEEYKVVVGEMAPAFAPCVIKPLDWTGPRFGGWHTEKMRRQMPLAKCKQQSVLSRLTKKQMPRVYSAVNALQAVEWRVNHRILSVANEARLRGLPLALPDREKLATPPCPVPHMYAELRGAELMSVLSESQKEAFRTWKADAAKTYNDEAERKAKYREVCATIDQGIQYKDFDKIHFVYTLDFRGRVYCQSSLISPQGGDLQKSLCQFANGMPLGESGVHWFKVQGANVWGWDKKQFEERDELVMADDFVELCLDIAADPLTFTDWTQADKPWQFLAWCFEYADFVSHCETNPPETFVSRIAVAMDGSCSGIQHYSAMLRDEIGGKEVNLVPGDRPQDIYGAVAKIVGRWMEDISELQVPCESFTRMLSKGAVEGQFKLCPLEAQMLSRAWVEIGVTRSMTKKPVMTLPYGSSQMTCRESMAQYLNDLQKKENEQSRAQNRKPVAVHPFNDSKGSMSRMQAELFASGLTWEAIGHVVVAARAAMRFIKKVTHETSSRNVALEWIAPTGFIVNQCIFDSTPRRVKTQMMGETFFTIREENPTISVNKMKSSAAPNFVHSMDASHLCIAVDEFALAGITSIAVIHDSFGTHAGNTERMRKLLINSFVDMYQNHDVIRDFYEAQCERVKCDLELDLPAVGTLDLETVRHSKYIFA

SEQ ID NO: 27 - protein, 1763 aa, synthetic construct
MAKRLQRCQDVNQVCEIYNSKGGIGELELRFDKLPQNLFAGVFDKLKPDGEIQTTMRVSNRDGVAREITFGGGVKTNEIFVKKQNICVFDVVDIFSYKVAVSTEETVVEKPTMETTAGVRFKIRLSVEDVVKDWRIDLTAVKTAELGKIAQHTASIVQRTFPDNLLKLTGAEVAKLAADSYELELEYTGKSPATNEKVNVAAKYAVELLSSVRNANSTAAASFGESVSDLCRVAKIIHTHEYANVVCRTPSFKMLLPQVVSLTKSSYYGGLYPPENLWLAGKTDGVRALVVCEDGVAKVITAESVDITHGVCSATTILDCELNVDAKILYVFDVIISNNTQVYTQPFSTRITTDISDIKIDGYKIEMKPFVKVVKADEATFKSAYKAPHNEGLIMIEDGAAYAATKTYKWKPLSHNTIDFLIKACPKQLINVDPYKPRAGYKLWLLFTTISLDQQRELGIEFIPAWKILFTDINMFGSRVPIQFQPAINPLAYVCYLPEDVNVNDGDIVEMRAVDGYDTIPKWELVRSRNDRKNEPGFYGNNYKIASDIYLNYIDVFHFEDLYKYNPGYFEKNKSDIYVAPNKYRRYLIKSLFGRYLRDAKWVIDAAAGRGADLHLYKAECVEHLLAIDIDPTAISELVRRRNEITGYNKSHRGGRNMHSHRGQSHCAKSTSLHALVADLRENPDVLIPKIIQSRPHERCYDAIVINFAIHYLCDTDEHIRDFLITVSRLLAPNGVFIFTTMDGESIVKLLADHKVRPGEAWTIHTGDVNSPDSTVPKYSIRRLYDSDKLTKTGQQIEVLLPMSGEMKAEPLCNIKNIISMARKMGLDLVESANFSVLYEAYARDYPDIYARMTPDDKLYNDLHTYAVFKRKKGGGGSPNIETQEALEAKMHGAGLDRFIRNNDRAITGGSASDTDWYRRLTRQFVKPMADGIQAYLDYYEGRRGKPSASLVHLRCLPTETASYIAIKTIFDGLTNPNQTAQNLSEQIGKRIEDEVRFMKLDNAAPKFIKAIKESLKRSSSQKYSHGHDVMVHAEKLLAEDKERQDKYGVMIDRHLSWPDNDIFNLGSKLIELFANNMLLDGEVLIQKRLIVRSQIDRVTVIEATPAMEKWIEEYKVVVGEMAPAFAPCVIKPLDWTGPRFGGWHTEKMRRQMPLAKCKQQSVLSRLTKKQMPRVYSAVNALQAVEWRVNHRILSVANEARLRGLPLALPDREKLATPPCPVPHMYAELRGAELMSVLSESQKEAFRTWKADAAKTYNDEAERKAKYREVCATIDQGIQYKDFDKIHFVYTLDFRGRVYCQSSLISPQGGDLQKSLCQFANGMPLGESGVHWFKVQGANVWGWDKKQFEERDELVMADDFVELCLDIAADPLTFTDWTQADKPWQFLAWCFEYADFVSHCETNPPETFVSRIAVAMDGSCSGIQHYSAMLRDEIGGKEVNLVPGDRPQDIYGAVAKIVGRWMEDISELQVPCESFTRMLSKGAVEGQFKLCPLEAQMLSRAWVEIGVTRSMTKKPVMTLPYGSSQMTCRESMAQYLNDLQKKENEQSRAQNRKPVAVHPFNDSKGSMSRMQAELFASGLTWEAIGHVVVAARAAMRFIKKVTHETSSRNVALEWIAPTGFIVNQCIFDSTPRRVKTQMMGETFFTIREENPTISVNKMKSSAAPNFVHSMDASHLCIAVDEFALAGITSIAVIHDSFGTHAGNTERMRKLLINSFVDMYQNHDVIRDFYEAQCERVKCDLELDLPAVGTLDLETVRHSKYIFA

SEQ ID NO: 28 - protein, 1770 aa, synthetic construct
MAKRLQRCQDVNQVCEIYNSKGGIGELELRFDKLPQNLFAGVFDKLKPDGEIQTTMRVSNRDGVAREITFGGGVKTNEIFVKKQNICVFDVVDIFSYKVAVSTEETVVEKPTMETTAGVRFKIRLSVEDVVKDWRIDLTAVKTAELGKIAQHTASIVQRTFPDNLLKLTGAEVAKLAADSYELELEYTGKSPATNEKVNVAAKYAVELLSSVRNANSTAAASFGESVSDLCRVAKIIHTHEYANVVCRTPSFKMLLPQVVSLTKSSYYGGLYPPENLWLAGKTDGVRALVVCEDGVAKVITAESVDITHGVCSATTILDCELNVDAKILYVFDVIISNNTQVYTQPFSTRITTDISDIKIDGYKIEMKPFVKVVKADEATFKSAYKAPHNEGLIMIEDGAAYAATKTYKWKPLSHNTIDFLIKACPKQLINVDPYKPRAGYKLWLLFTTISLDQQRELGIEFIPAWKILFTDINMFGSRVPIQFQPAINPLAYVCYLPEDVNVNDGDIVEMRAVDGYDTIPKWELVRSRNDRKNEPGFYGNNYKIASDIYLNYIDVFHFEDLYKYNPGYFEKNKSDIYVAPNKYRRYLIKSLFGRYLRDAKWVIDAAAGRGADLHLYKAECVEHLLAIDIDPTAISELVRRRNEITGYNKSHRGGRNMHSHRGQSHCAKSTSLHALVADLRENPDVLIPKIIQSRPHERCYDAIVINFAIHYLCDTDEHIRDFLITVSRLLAPNGVFIFTTMDGESIVKLLADHKVRPGEAWTIHTGDVNSPDSTVPKYSIRRLYDSDKLTKTGQQIEVLLPMSGEMKAEPLCNIKNIISMARKMGLDLVESANFSVLYEAYARDYPDIYARMTPDDKLYNDLHTYAVFKRKKGGGGSSGGGGSSPNIETQEALEAKMHGAGLDRFIRNNDRAITGGSASDTDWYRRLTRQFVKPMADGIQAYLDYYEGRRGKPSASLVHLRCLPTETASYIAIKTIFDGLTNPNQTAQNLSEQIGKRIEDEVRFMKLDNAAPKFIKAIKESLKRSSSQKYSHGHDVMVHAEKLLAEDKERQDKYGVMIDRHLSWPDNDIFNLGSKLIELFANNMLLDGEVLIQKRLIVRSQIDRVTVIEATPAMEKWIEEYKVVVGEMAPAFAPCVIKPLDWTGPRFGGWHTEKMRRQMPLAKCKQQSVLSRLTKKQMPRVYSAVNALQAVEWRVNHRILSVANEARLRGLPLALPDREKLATPPCPVPHMYAELRGAELMSVLSESQKEAFRTWKADAAKTYNDEAERKAKYREVCATIDQGIQYKDFDKIHFVYTLDFRGRVYCQSSLISPQGGDLQKSLCQFANGMPLGESGVHWFKVQGANVWGWDKKQFEERDELVMADDFVELCLDIAADPLTFTDWTQADKPWQFLAWCFEYADFVSHCETNPPETFVSRIAVAMDGSCSGIQHYSAMLRDEIGGKEVNLVPGDRPQDIYGAVAKIVGRWMEDISELQVPCESFTRMLSKGAVEGQFKLCPLEAQMLSRAWVEIGVTRSMTKKPVMTLPYGSSQMTCRESMAQYLNDLQKKENEQSRAQNRKPVAVHPFNDSKGSMSRMQAELFASGLTWEAIGHVVVAARAAMRFIKKVTHETSSRNVALEWIAPTGFIVNQCIFDSTPRRVKTQMMGETFFTIREENPTISVNKMKSSAAPNFVHSMDASHLCIAVDEFALAGITSIAVIHDSFGTHAGNTERMRKLLINSFVDMYQNHDVIRDFYEAQCERVKCDLELDLPAVGTLDLETVRHSKYIFA

SEQ ID NO: 29 - protein, 1779 aa, synthetic construct
MAKRLQRCQDVNQVCEIYNSKGGIGELELRFDKLPQNLFAGVFDKLKPDGEIQTTMRVSNRDGVAREITFGGGVKTNEIFVKKQNICVFDVVDIFSYKVAVSTEETVVEKPTMETTAGVRFKIRLSVEDVVKDWRIDLTAVKTAELGKIAQHTASIVQRTFPDNLLKLTGAEVAKLAADSYELELEYTGKSPATNEKVNVAAKYAVELLSSVRNANSTAAASFGESVSDLCRVAKIIHTHEYANVVCRTPSFKMLLPQVVSLTKSSYYGGLYPPENLWLAGKTDGVRALVVCEDGVAKVITAESVDITHGVCSATTILDCELNVDAKILYVFDVIISNNTQVYTQPFSTRITTDISDIKIDGYKIEMKPFVKVVKADEATFKSAYKAPHNEGLIMIEDGAAYAATKTYKWKPLSHNTIDFLIKACPKQLINVDPYKPRAGYKLWLLFTTISLDQQRELGIEFIPAWKILFTDINMFGSRVPIQFQPAINPLAYVCYLPEDVNVNDGDIVEMRAVDGYDTIPKWELVRSRNDRKNEPGFYGNNYKIASDIYLNYIDVFHFEDLYKYNPGYFEKNKSDIYVAPNKYRRYLIKSLFGRYLRDAKWVIDAAAGRGADLHLYKAECVEHLLAIDIDPTAISELVRRRNEITGYNKSHRGGRNMHSHRGQSHCAKSTSLHALVADLRENPDVLIPKIIQSRPHERCYDAIVINFAIHYLCDTDEHIRDFLITVSRLLAPNGVFIFTTMDGESIVKLLADHKVRPGEAWTIHTGDVNSPDSTVPKYSIRRLYDSDKLTKTGQQIEVLLPMSGEMKAEPLCNIKNIISMARKMGLDLVESANFSVLYEAYARDYPDIYARMTPDDKLYNDLHTYAVFKRKKGSSAGSAALINKERATETHERPNIETQEALEAKMHGAGLDRFIRNNDRAITGGSASDTDWYRRLTRQFVKPMADGIQAYLDYYEGRRGKPSASLVHLRCLPTETASYIAIKTIFDGLTNPNQTAQNLSEQIGKRIEDEVRFMKLDNAAPKFIKAIKESLKRSSSQKYSHGHDVMVHAEKLLAEDKERQDKYGVMIDRHLSWPDNDIFNLGSKLIELFANNMLLDGEVLIQKRLIVRSQIDRVTVIEATPAMEKWIEEYKVVVGEMAPAFAPCVIKPLDWTGPRFGGWHTEKMRRQMPLAKCKQQSVLSRLTKKQMPRVYSAVNALQAVEWRVNHRILSVANEARLRGLPLALPDREKLATPPCPVPHMYAELRGAELMSVLSESQKEAFRTWKADAAKTYNDEAERKAKYREVCATIDQGIQYKDFDKIHFVYTLDFRGRVYCQSSLISPQGGDLQKSLCQFANGMPLGESGVHWFKVQGANVWGWDKKQFEERDELVMADDFVELCLDIAADPLTFTDWTQADKPWQFLAWCFEYADFVSHCETNPPETFVSRIAVAMDGSCSGIQHYSAMLRDEIGGKEVNLVPGDRPQDIYGAVAKIVGRWMEDISELQVPCESFTRMLSKGAVEGQFKLCPLEAQMLSRAWVEIGVTRSMTKKPVMTLPYGSSQMTCRESMAQYLNDLQKKENEQSRAQNRKPVAVHPFNDSKGSMSRMQAELFASGLTWEAIGHVVVAARAAMRFIKKVTHETSSRNVALEWIAPTGFIVNQCIFDSTPRRVKTQMMGETFFTIREENPTISVNKMKSSAAPNFVHSMDASHLCIAVDEFALAGITSIAVIHDSFGTHAGNTERMRKLLINSFVDMYQNHDVIRDFYEAQCERVKCDLELDLPAVGTLDLETVRHSKYIFA

SEQ ID NO: 30 - DNA, 7921 bp, synthetic construct
  source 1..7921: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcaccctaatattgaaactcaggaagcgctggaggccaaaatgcatggagctgggttggaccgcttcatccgtaacaacgaccgtgctattaccgggggaagtgcttctgatacggactggtatcgtcgcttaacacgtcaattcgttaagccaatggcagatggaattcaagcttacctggattattatgagggccgtcgtggtaagccttcggccagcctggtccatcttcgctgtttgcctacagaaaccgcatcttatattgctatcaaaactatcttcgatggattgacgaaccccaatcagaccgcgcaaaacttaagtgagcagattggaaagcgcatcgaagatgaagttcgcttcatgaagcttgataatgcagccccaaaattcattaaggctatcaaggagtcgttaaaacgctcatcatctcagaaatactcgcatggccatgatgtgatggtgcatgctgaaaagctgttggcggaagacaaagagcgtcaagacaagtatggggtcatgattgatcgccacttatcctggcctgataatgacatttttaatcttggttccaagttaattgagttatttgcaaacaacatgctgttggatggagaagtactgatccaaaaacgccttatcgtccgctctcaaatcgatcgtgtaacagttatcgaggcaacgcctgctatggagaagtggattgaggagtacaaggttgtcgttggcgaaatggctcccgccttcgcaccatgtgtcattaagccactggattggacaggacctcgtttcggaggatggcacactgagaaaatgcgtcgtcaaatgccacttgccaagtgcaaacaacaatcggttcttagtcgcttgactaagaaacagatgccgcgtgtttactcagccgtaaatgctctgcaagcggtggaatggcgcgttaatcaccgtattttgtctgtcgcgaatgaggctcgcctgcgtggtttgccgttagccttaccggaccgtgaaaagcttgcaactccgccatgcccggttcctcacatgtatgcggagcttcgtggtgcagaactgatgagtgtcttaagtgaatcccagaaggaagcgtttcgtacttggaaagcggatgcggcaaagacttacaacgatgaagctgagcgcaaggcaaaatatcgcgaagtgtgcgcgacgatcgatcaaggaattcagtacaaggatttcgacaagattcatttcgtttatacattagactttcgcgggcgcgtgtactgtcagagttctttgatcagcccacagggtggggacttgcaaaagtccttgtgccagtttgccaatggtatgcccttaggagaatcgggagttcactggttcaaggtccaaggagcgaatgtctggggctgggataaaaaacaatttgaagagcgtgacgaattagtgatggctgacgattttgtagagctttgcttagacattgcggcggaccccctgactttcactgactggacgcaagccgacaagccgtggcaattcttagcgtggtgttttgagtatgcagactttgtttcacactgtgagactaatcctccagagacctttgtctcacgtattgcggtcgcgatggacggatcatgttcgggcatccagcactactccgctatgttgcgcgacgagattggaggcaaggaagttaatttggtacctggggaccgtccccaagatatctatggagctgttgccaagatcgtcggacgttggatggaggatatctcagagcttcaggtgccctgtgaatcattcacgcgtatgttgtcgaaaggtgcagtggaagggcaatttaagttgtgccctttggaggcgcagatgctgtcccgcgcttgggtcgaaatcggggtgactcgcagcatgaccaagaaaccagtcatgacgcttccgtacggaagctcacaaatgacatgtcgcgaaagcatggctcagtatctgaatgatttacagaagaaggagaatgaacaatcacgcgctcaaaatcgcaaacccgttgcggtacatccttttaatgattcaaaaggctccatgtctcgtatgcaagccgagttatttgcttccggtcttacatgggaggctatcgggcacgtggtagtagccgcgcgtgcggcgatgcgcttcatcaagaaagtcacacatgaaacttcgtcgcgtaacgtcgcgttggaatggattgcgccaactggttttatcgtcaatcaatgcatttttgactccacccctcgccgtgtcaaaacgcagatgatgggtgagacgttttttaccattcgcgaggaaaacccaacaattagtgtaaacaagatgaaatcatccgcagctcctaacttcgtgcacagcatggacgcgtctcacttgtgcattgcagttgacgagttcgcattggccggaatcacctctattgccgtcattcacgactccttcggcacccatgcgggaaataccgaacgtatgcgtaagttacttattaacagctttgttgatatgtaccaaaaccatgacgttattcgcgacttttatgaggcacaatgcgaacgtgtgaagtgcgatcttgaactggatttacctgcggtgggcaccctggacctggaaactgtgcgccacagtaagtatatctttgcctaactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 31 - DNA, 19 bp, synthetic construct
  source 1..19: mol_type = other DNA
aattaagttgcattataga

SEQ ID NO: 32 - DNA, 19 bp, synthetic construct
  source 1..19: mol_type = other DNA
gtttaagttgcattataga

SEQ ID NO: 33 - DNA, 19 bp, synthetic construct
  source 1..19: mol_type = other DNA
ttttaagttgcattatagg

SEQ ID NO: 34 - DNA, 19 bp, synthetic construct
  source 1..19: mol_type = other DNA
gattaggttgcattatagg

SEQ ID NO: 35 - DNA, 19 bp, synthetic construct
  source 1..19: mol_type = other DNA
gattaagttgcattaaagg

SEQ ID NO: 36 - DNA, 19 bp, synthetic construct
  source 1..19: mol_type = other DNA
atttaggttgcattagagg

SEQ ID NO: 37 - DNA, 19 bp, synthetic construct
  source 1..19: mol_type = other DNA
atttaagttgcattataga

SEQ ID NO: 38 - DNA, 19 bp, synthetic construct
  source 1..19: mol_type = other DNA
atttaagttgcattatagg

SEQ ID NO: 39 - DNA, 16 bp, synthetic construct
  source 1..16: mol_type = other DNA
ttaagttgcattatag

SEQ ID NO: 40 - DNA, 16 bp, synthetic construct
  source 1..16: mol_type = other DNA
ttaggttgcattatag

SEQ ID NO: 41 - DNA, 16 bp, synthetic construct
  source 1..16: mol_type = other DNA
ttaggttgcattagag

SEQ ID NO: 42 - DNA, 19 bp, synthetic construct
  source 1..19: mol_type = other DNA
ttttaagttgcattatagg

SEQ ID NO: 43 - DNA, 19 bp, synthetic construct
  source 1..19: mol_type = other DNA
ttttaagttgcattatagg

SEQ ID NO: 44 - DNA, 19 bp, synthetic construct
  source 1..19: mol_type = other DNA
ttttaagttgcattatagt

SEQ ID NO: 45 - DNA, 19 bp, synthetic construct
  source 1..19: mol_type = other DNA
ttttaagttgcattataag

SEQ ID NO: 46 - DNA, 19 bp, synthetic construct
  source 1..19: mol_type = other DNA
ttttaagttgcattataat

SEQ ID NO: 47 - DNA, 4083 bp, synthetic construct
  source 1..4083: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggctagccccgcgaaatgtttaagttgcattatagggaaataagagagaaaagaagagtaagaagaaatataagagccaccgccaccatggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaaaagtctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtgaggcgcgcctatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaaaaaaaaaaaaaaaaaaaaaagaagagcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 48 - DNA, 4083 bp, synthetic construct
  source 1..4083: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggctagccccgcgaaattaatacgactcactatagggaaataagagagaaaagaagagtaagaagaaatataagagccaccgccaccatggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaaaagtctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtgaggcgcgcctatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaaaaaaaaaaaaaaaaaaaaaagaagagcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 49 - RNA, 2101 nt, synthetic construct
  source 1..2101: mol_type = other RNA
gggaaataagagagaaaagaagagtaagaagaaatataagagccaccgccaccatggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaaaagtctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtgaggcgcgcctatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaaaaaaaaaaaaaaaaaa

SEQ ID NO: 50 - RNA, 2062 nt, synthetic construct
  source 1..2062: mol_type = other RNA
gggaaataagagagaaaagaagagtaagaagaaatataagaccccggcgccgccaccatggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaaaagtctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaaaaaaaaaaaaaaaaaaaaaaaaaaaaa

SEQ ID NO: 51 - DNA, 1823 bp, synthetic construct
  source 1..1823: mol_type = other DNA
ttttaagttgcattatagggagacccaagcttggtaccgagctcggatccgccaccatgaagaccttaattcttgccgttgcattagtctactgcgccactgttcattgccaggactgtccttacgaacctgatccaccaaacacagttccaacttcctgtgaagctaaagaaggagaatgtattgatagcagctgtggcacctgcacgagagacatactatcagatggactgtgtgaaaataaaccaggaaaaacatgttgccgaatgtgtcagtatgtaattgaatgcagagtagaggccgcaggatggtttagaacattctatggaaagagattccagttccaggaacctggtacatacgtgttgggtcaaggaaccaagggcggcgactggaaggtgtccatcaccctggagaacctggatggaaccaagggggctgtgctgaccaagacaagactggaagtggctggagacatcattgacatcgctcaagctactgagaatcccatcactgtaaacggtggagctgaccctatcatcgccaacccgtacaccatcggcgaggtcaccatcgctgttgttgagatgccaggcttcaacatcaccgtcattgagttcttcaaactgatcgtgatcgacatcctcggaggaagatctgtaagaatcgccccagacacagcaaacaaaggaatgatctctggcctctgtggagatcttaaaatgatggaagatacagacttcacttcagatccagaacaactcgctattcagcctaagatcaaccaggagtttgacggttgtccactctatggaaatcctgatgacgttgcatactgcaaaggtcttctggagccgtacaaggacagctgccgcaaccccatcaacttctactactacaccatctcctgcgccttcgcccgctgtatgggtggagacgagcgagcctcacacgtgctgcttgactacagggagacgtgcgctgctcccgaaactagaggaacctgcgttttgtctggacatactttctacgatacatttgacaaagcaagataccaattccagggtccctgcaaggagattcttatggccgccgactgtttctggaacacttgggatgtgaaggtttcacacaggaatgttgactcttacactgaagtagagaaagtacgaatcaggaaacaatcgactgtagtagaactcattgttgatggaaaacagattctggttggaggagaagccgtgtccgtcccgtacagctctcagaacacttccatctactggcaagatggtgacatactgactacagccatcctacctgaagctctggtggtcaagttcaacttcaagcaactgctcgtcgtacatattagagatccattcgatggtaagacttgcggtatttgcggtaactacaaccaggatttcagtgatgattcttttgatgctgaaggagcctgtgatctgacccccaacccaccgggatgcaccgaagaacagaaacctgaagctgaacgactctgcaatagtctcttcgccggtcaaagtgatcttgatcagaaatgtaacgtgtgccacaagcctgaccgtgtcgaacgatgcatgtacgagtattgcctgaggggacaacagggtttctgtgaccacgcatgggagttcaagaaagaatgctacataaagcatggagacaccctagaagtaccagatgaatgcaaataggcggccgcaataaaatatctttattttcattacatctgtgtgttggttttttgtgtgtctagaaataattcttactgtcatgccaagtaagatgcttttctgtgc

SEQ ID NO: 52 - RNA, 1806 nt, synthetic construct
  source 1..1806: mol_type = other RNA
gggagacccaagcttggtaccgagctcggatccgccaccatgaagaccttaattcttgccgttgcattagtctactgcgccactgttcattgccaggactgtccttacgaacctgatccaccaaacacagttccaacttcctgtgaagctaaagaaggagaatgtattgatagcagctgtggcacctgcacgagagacatactatcagatggactgtgtgaaaataaaccaggaaaaacatgttgccgaatgtgtcagtatgtaattgaatgcagagtagaggccgcaggatggtttagaacattctatggaaagagattccagttccaggaacctggtacatacgtgttgggtcaaggaaccaagggcggcgactggaaggtgtccatcaccctggagaacctggatggaaccaagggggctgtgctgaccaagacaagactggaagtggctggagacatcattgacatcgctcaagctactgagaatcccatcactgtaaacggtggagctgaccctatcatcgccaacccgtacaccatcggcgaggtcaccatcgctgttgttgagatgccaggcttcaacatcaccgtcattgagttcttcaaactgatcgtgatcgacatcctcggaggaagatctgtaagaatcgccccagacacagcaaacaaaggaatgatctctggcctctgtggagatcttaaaatgatggaagatacagacttcacttcagatccagaacaactcgctattcagcctaagatcaaccaggagtttgacggttgtccactctatggaaatcctgatgacgttgcatactgcaaaggtcttctggagccgtacaaggacagctgccgcaaccccatcaacttctactactacaccatctcctgcgccttcgcccgctgtatgggtggagacgagcgagcctcacacgtgctgcttgactacagggagacgtgcgctgctcccgaaactagaggaacctgcgttttgtctggacatactttctacgatacatttgacaaagcaagataccaattccagggtccctgcaaggagattcttatggccgccgactgtttctggaacacttgggatgtgaaggtttcacacaggaatgttgactcttacactgaagtagagaaagtacgaatcaggaaacaatcgactgtagtagaactcattgttgatggaaaacagattctggttggaggagaagccgtgtccgtcccgtacagctctcagaacacttccatctactggcaagatggtgacatactgactacagccatcctacctgaagctctggtggtcaagttcaacttcaagcaactgctcgtcgtacatattagagatccattcgatggtaagacttgcggtatttgcggtaactacaaccaggatttcagtgatgattcttttgatgctgaaggagcctgtgatctgacccccaacccaccgggatgcaccgaagaacagaaacctgaagctgaacgactctgcaatagtctcttcgccggtcaaagtgatcttgatcagaaatgtaacgtgtgccacaagcctgaccgtgtcgaacgatgcatgtacgagtattgcctgaggggacaacagggtttctgtgaccacgcatgggagttcaagaaagaatgctacataaagcatggagacaccctagaagtaccagatgaatgcaaataggcggccgcaataaaatatctttattttcattacatctgtgtgttggttttttgtgtgtctagaaataattcttactgtcatgccaagtaagatgcttttctgtgc

SEQ ID NO: 53 - DNA, 3294 bp, synthetic construct
  source 1..3294: mol_type = other DNA
gctagtggtgctagccccgcgaaatgtttaagttgcattatagggtctagaaataattttgtttaactttagagtacacgagtcaggctacagcatcctctggttcagactacttgattcatgtgtaccctatatgcgaggatatgtgtatcgtagaaattgtcaggcagtaacgttccgcgagttttaatgggcgcgccatgactctaagagtgatatacctcctcggtctcgggcccggggtgtaattagcccagttagacacgatcgcccgacgtatattgttgcttgggtatcgtcgcatgcgaagtattgcccaaggagacacaacaagcaacttatgttgactcccttcgaccattaaaatttgttagaacggacagaaaggatgcgccttataaatgtcctgtgcagtgatgaagcgacctcaaaacgcttcatgatctaaccgactcaccttgccgttccctccgcgccttaaaaccggccggtcttgcgaaaagcgggaaacgagtttacccacggatagcagggaatgttgcggctggctagggagcatgaaggtagatactccacggcttacctttccggggctcaacatctagccacagaccttttcgttaagcccacccccactggatactgaatcatcagggaaccggacccaaccagtttgggctcgtccaagcttcggtctcgtccctaagtgcaaagatatggaaagagcagcataggtatatggattattcttttaccactcgtttcttaccgtaacttacgcaatggatcacgtgccgaggcggcggtacagctgttcgaagggctctgtcgggaacgctaacatccagccggtaaattccaaactagggaaaggacacgcactgaattgaatatagtcgtgaagggtggtgtaagtcgtgcacagcccgcattaagtactaaacagcgtccaatcttgatctacttacggcctgatgttcttcagcacctcctagcactggagtacttcgctatcaatgagattagcactttgtacatgtcatccagcccgagtctggggtccgacaatgcggtcgccgattggtatctgcatgtagtattaaacggagctgccgcggctgcggattatagttcatgtcttgacggtcctcgtgaactgtggttaacctgcaggaggcctttaattaaggtggtgcggccgcgctagcggtcccgggggatcgatccggctgctaacaaagcccgaaaggaagctgagttggctgctgccaccgctgagcaataactagcataaccccttggggcctctaaacgggtcttgaggggttttttgctgaaaggaggaactatatccggaagcttggcactggccgaccggggtcgagcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccgctaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacagatccgggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagt

SEQ ID NO: 54 - DNA, 3294 bp, synthetic construct
  source 1..3294: mol_type = other DNA
gctagtggtgctagccccgcgaaattaatacgactcactatagggtctagaaataattttgtttaactttagagtacacgagtcaggctacagcatcctctggttcagactacttgattcatgtgtaccctatatgcgaggatatgtgtatcgtagaaattgtcaggcagtaacgttccgcgagttttaatgggcgcgccatgactctaagagtgatatacctcctcggtctcgggcccggggtgtaattagcccagttagacacgatcgcccgacgtatattgttgcttgggtatcgtcgcatgcgaagtattgcccaaggagacacaacaagcaacttatgttgactcccttcgaccattaaaatttgttagaacggacagaaaggatgcgccttataaatgtcctgtgcagtgatgaagcgacctcaaaacgcttcatgatctaaccgactcaccttgccgttccctccgcgccttaaaaccggccggtcttgcgaaaagcgggaaacgagtttacccacggatagcagggaatgttgcggctggctagggagcatgaaggtagatactccacggcttacctttccggggctcaacatctagccacagaccttttcgttaagcccacccccactggatactgaatcatcagggaaccggacccaaccagtttgggctcgtccaagcttcggtctcgtccctaagtgcaaagatatggaaagagcagcataggtatatggattattcttttaccactcgtttcttaccgtaacttacgcaatggatcacgtgccgaggcggcggtacagctgttcgaagggctctgtcgggaacgctaacatccagccggtaaattccaaactagggaaaggacacgcactgaattgaatatagtcgtgaagggtggtgtaagtcgtgcacagcccgcattaagtactaaacagcgtccaatcttgatctacttacggcctgatgttcttcagcacctcctagcactggagtacttcgctatcaatgagattagcactttgtacatgtcatccagcccgagtctggggtccgacaatgcggtcgccgattggtatctgcatgtagtattaaacggagctgccgcggctgcggattatagttcatgtcttgacggtcctcgtgaactgtggttaacctgcaggaggcctttaattaaggtggtgcggccgcgctagcggtcccgggggatcgatccggctgctaacaaagcccgaaaggaagctgagttggctgctgccaccgctgagcaataactagcataaccccttggggcctctaaacgggtcttgaggggttttttgctgaaaggaggaactatatccggaagcttggcactggccgaccggggtcgagcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccgctaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacagatccgggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagt

SEQ ID NO: 55 - RNA, 1118 nt, synthetic construct
  source 1..1118: mol_type = other RNA
gggtctagaaataattttgtttaactttagagtacacgagtcaggctacagcatcctctggttcagactacttgattcatgtgtaccctatatgcgaggatatgtgtatcgtagaaattgtcaggcagtaacgttccgcgagttttaatgggcgcgccatgactctaagagtgatatacctcctcggtctcgggcccggggtgtaattagcccagttagacacgatcgcccgacgtatattgttgcttgggtatcgtcgcatgcgaagtattgcccaaggagacacaacaagcaacttatgttgactcccttcgaccattaaaatttgttagaacggacagaaaggatgcgccttataaatgtcctgtgcagtgatgaagcgacctcaaaacgcttcatgatctaaccgactcaccttgccgttccctccgcgccttaaaaccggccggtcttgcgaaaagcgggaaacgagtttacccacggatagcagggaatgttgcggctggctagggagcatgaaggtagatactccacggcttacctttccggggctcaacatctagccacagaccttttcgttaagcccacccccactggatactgaatcatcagggaaccggacccaaccagtttgggctcgtccaagcttcggtctcgtccctaagtgcaaagatatggaaagagcagcataggtatatggattattcttttaccactcgtttcttaccgtaacttacgcaatggatcacgtgccgaggcggcggtacagctgttcgaagggctctgtcgggaacgctaacatccagccggtaaattccaaactagggaaaggacacgcactgaattgaatatagtcgtgaagggtggtgtaagtcgtgcacagcccgcattaagtactaaacagcgtccaatcttgatctacttacggcctgatgttcttcagcacctcctagcactggagtacttcgctatcaatgagattagcactttgtacatgtcatccagcccgagtctggggtccgacaatgcggtcgccgattggtatctgcatgtagtattaaacggagctgccgcggctgcggattatagttcatgtcttgacggtcctcgtgaactgt

SEQ ID NO: 56 - DNA, 3296 bp, synthetic construct
  source 1..3296: mol_type = other DNA
gctagtggtgctagccccgcgaaatgtttaagttgcattatagggtctagaaataattttgtttaactttagagtacacgagtcaggctacagcatcttgacaccagaatattatggattggacgcttcccactaaatggaagactgttcggtcataaacactactaggaattcctctccagtcatcatgttcgatcgtctagcagcaatctcttccgatcgatatttgcgcgtgactcaggcgagcccatgacagcttctccccgtgagaaccacgactagaagttatctgttgagctgctagcttcgtggcccggccatggtagtagcggctcactcgcgctaactttgcctgctcgagaaaacgggcgaaacacccagcaacacaagccacttaatttgttgatagataataagatcaggttattagtcgctctgcacttactttaagtgccaactatgctgtatcggccagggtgaaaacgggtgccgccacttcagtgtgtcggagtctgctgacggattagggcacagacgtatggttatatcctaaggtagtgtgtcaatgtactggggacaaagtcagtgggcaccgcatcaggagtgcaacctccgctagtaccgactcgtcaatgctttgagcgatggcttgcgctcccaaatccttaagcttttatgcattcggctctggccctcaggcctgacctggaatttcatcggaaacgccttaaccgacattacatcgacaccaagatcccgacgcttcatgcggagacgatagagactctaaccaagaataaaaggagtagtccctaatctactgaaacggggatacctcaaatcacgggaatgcgttactgacccgctatgtgaggctcggatcaccctcgttctattgccttgtaatcatggtggggcggcggagcgggattagagggtgtccctaatgtgagtagatctgtagtaatgatacgtctcctcaatatgaggcgtattgcaggtcacagcacagggagatttcggcgcacccagccgagttgcctccgtcgttgtttaggtatatgcataactgctcacgacaaatacagcagagcctacgttgggttatcgaatccttgtggacaagaagcttcttcatgtcttgacggtcctcgtgaactgtggttaacctgcaggaggcctttaattaaggtggtgcggccgcgctagcggtcccgggggatcgatccggctgctaacaaagcccgaaaggaagctgagttggctgctgccaccgctgagcaataactagcataaccccttggggcctctaaacgggtcttgaggggttttttgctgaaaggaggaactatatccggaagcttggcactggccgaccggggtcgagcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccgctaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacagatccgggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagt

SEQ ID NO: 57 - DNA, 3296 bp, synthetic construct
  source 1..3296: mol_type = other DNA
gctagtggtgctagccccgcgaaattaatacgactcactatagggtctagaaataattttgtttaactttagagtacacgagtcaggctacagcatcttgacaccagaatattatggattggacgcttcccactaaatggaagactgttcggtcataaacactactaggaattcctctccagtcatcatgttcgatcgtctagcagcaatctcttccgatcgatatttgcgcgtgactcaggcgagcccatgacagcttctccccgtgagaaccacgactagaagttatctgttgagctgctagcttcgtggcccggccatggtagtagcggctcactcgcgctaactttgcctgctcgagaaaacgggcgaaacacccagcaacacaagccacttaatttgttgatagataataagatcaggttattagtcgctctgcacttactttaagtgccaactatgctgtatcggccagggtgaaaacgggtgccgccacttcagtgtgtcggagtctgctgacggattagggcacagacgtatggttatatcctaaggtagtgtgtcaatgtactggggacaaagtcagtgggcaccgcatcaggagtgcaacctccgctagtaccgactcgtcaatgctttgagcgatggcttgcgctcccaaatccttaagcttttatgcattcggctctggccctcaggcctgacctggaatttcatcggaaacgccttaaccgacattacatcgacaccaagatcccgacgcttcatgcggagacgatagagactctaaccaagaataaaaggagtagtccctaatctactgaaacggggatacctcaaatcacgggaatgcgttactgacccgctatgtgaggctcggatcaccctcgttctattgccttgtaatcatggtggggcggcggagcgggattagagggtgtccctaatgtgagtagatctgtagtaatgatacgtctcctcaatatgaggcgtattgcaggtcacagcacagggagatttcggcgcacccagccgagttgcctccgtcgttgtttaggtatatgcataactgctcacgacaaatacagcagagcctacgttgggttatcgaatccttgtggacaagaagcttcttcatgtcttgacggtcctcgtgaactgtggttaacctgcaggaggcctttaattaaggtggtgcggccgcgctagcggtcccgggggatcgatccggctgctaacaaagcccgaaaggaagctgagttggctgctgccaccgctgagcaataactagcataaccccttggggcctctaaacgggtcttgaggggttttttgctgaaaggaggaactatatccggaagcttggcactggccgaccggggtcgagcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccgctaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacagatccgggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagt

SEQ ID NO: 58 - RNA, 1120 nt, synthetic construct
  source 1..1120: mol_type = other RNA
gggtctagaaataattttgtttaactttagagtacacgagtcaggctacagcatcttgacaccagaatattatggattggacgcttcccactaaatggaagactgttcggtcataaacactactaggaattcctctccagtcatcatgttcgatcgtctagcagcaatctcttccgatcgatatttgcgcgtgactcaggcgagcccatgacagcttctccccgtgagaaccacgactagaagttatctgttgagctgctagcttcgtggcccggccatggtagtagcggctcactcgcgctaactttgcctgctcgagaaaacgggcgaaacacccagcaacacaagccacttaatttgttgatagataataagatcaggttattagtcgctctgcacttactttaagtgccaactatgctgtatcggccagggtgaaaacgggtgccgccacttcagtgtgtcggagtctgctgacggattagggcacagacgtatggttatatcctaaggtagtgtgtcaatgtactggggacaaagtcagtgggcaccgcatcaggagtgcaacctccgctagtaccgactcgtcaatgctttgagcgatggcttgcgctcccaaatccttaagcttttatgcattcggctctggccctcaggcctgacctggaatttcatcggaaacgccttaaccgacattacatcgacaccaagatcccgacgcttcatgcggagacgatagagactctaaccaagaataaaaggagtagtccctaatctactgaaacggggatacctcaaatcacgggaatgcgttactgacccgctatgtgaggctcggatcaccctcgttctattgccttgtaatcatggtggggcggcggagcgggattagagggtgtccctaatgtgagtagatctgtagtaatgatacgtctcctcaatatgaggcgtattgcaggtcacagcacagggagatttcggcgcacccagccgagttgcctccgtcgttgtttaggtatatgcataactgctcacgacaaatacagcagagcctacgttgggttatcgaatccttgtggacaagaagcttcttcatgtcttgacggtcctcgtgaactgt

SEQ ID NO: 59 - DNA, 11580 bp, synthetic construct
  source 1..11580: mol_type = other DNA
ttttaagttgcattataggattgacggcgtagtacacactattgaatcaaacagccgaccaattgcactaccatcacaatggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatacgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccaaggctgcttttgaggatgctcaggaggaagccagagcggagaagctccgagaagcacttccaccattagtggcagacaaaggcatcgaggcagccgcagaagttgtctgcgaagtggaggggctccaggcggacatcggagcagcattagttgaaaccccgcgcggtcacgtaaggataatacctcaagcaaatgaccgtatgatcggacagtatatcgttgtctcgccaaactctgtgctgaagaatgccaaactcgcaccagcgcacccgctagcagatcaggttaagatcataacacactccggaagatcaggaaggtacgcggtcgaaccatacgacgctaaagtactgatgccagcaggaggtgccgtaccatggccagaattcctagcactgagtgagagcgccacgttagtgtacaacgaaagagagtttgtgaaccgcaaactataccacattgccatgcatggccccgccaagaatacagaagaggagcagtacaaggttacaaaggcagagcttgcagaaacagagtacgtgtttgacgtggacaagaagcgttgcgttaagaaggaagaagcctcaggtctggtcctctcgggagaactgaccaaccctccctatcatgagctagctctggagggactgaagacccgacctgcggtcccgtacaaggtcgaaacaataggagtgataggcacaccggggtcgggcaagtcagctattatcaagtcaactgtcacggcacgagatcttgttaccagcggaaagaaagaaaattgtcgcgaaattgaggccgacgtgctaagactgaggggtatgcagattacgtcgaagacagtagattcggttatgctcaacggatgccacaaagccgtagaagtgctgtacgttgacgaagcgttcgcgtgccacgcaggagcactacttgccttgattgctatcgtcaggccccgcaagaaggtagtactatgcggagaccccatgcaatgcggattcttcaacatgatgcaactaaaggtacatttcaatcaccctgaaaaagacatatgcaccaagacattctacaagtatatctcccggcgttgcacacagccagttacagctattgtatcgacactgcattacgatggaaagatgaaaaccacgaacccgtgcaagaagaacattgaaatcgatattacaggggccacaaagccgaagccaggggatatcatcctgacatgtttccgcgggtgggttaagcaattgcaaatcgactatcccggacatgaagtaatgacagccgcggcctcacaagggctaaccagaaaaggagtgtatgccgtccggcaaaaagtcaatgaaaacccactgtacgcgatcacatcagagcatgtgaacgtgttgctcacccgcactgaggacaggctagtgtggaaaaccttgcagggcgacccatggattaagcagcccactaacatacctaaaggaaactttcaggctactatagaggactgggaagctgaacacaagggaataattgctgcaataaacagccccactccccgtgccaatccgttcagctgcaagaccaacgtttgctgggcgaaagcattggaaccgatactagccacggccggtatcgtacttaccggttgccagtggagcgaactgttcccacagtttgcggatgacaaaccacattcggccatttacgccttagacgtaatttgcattaagtttttcggcatggacttgacaagcggactgttttctaaacagagcatcccactaacgtaccatcccgccgattcagcgaggccggtagctcattgggacaacagcccaggaacccgcaagtatgggtacgatcacgccattgccgccgaactctcccgtagatttccggtgttccagctagctgggaagggcacacaacttgatttgcagacggggagaaccagagttatctctgcacagcataacctggtcccggtgaaccgcaatcttcctcacgccttagtccccgagtacaaggagaagcaacccggcccggtcaaaaaattcttgaaccagttcaaacaccactcagtacttgtggtatcagaggaaaaaattgaagctccccgtaagagaatcgaatggatcgccccgattggcatagccggtgcagataagaactacaacctggctttcgggtttccgccgcaggcacggtacgacctggtgttcatcaacattggaactaaatacagaaaccaccactttcagcagtgcgaagaccatgcggcgaccttaaaaaccctttcgcgttcggccctgaattgccttaacccaggaggcaccctcgtggtgaagtcctatggctacgccgaccgcaacagtgaggacgtagtcaccgctcttgccagaaagtttgtcagggtgtctgcagcgagaccagattgtgtctcaagcaatacagaaatgtacctgattttccgacaactagacaacagccgtacacggcaattcaccccgcaccatctgaattgcgtgatttcgtccgtgtatgagggtacaagagatggagttggagccgcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccccgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggatccatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttccactacgcaagcagagacgtagacgcaggagcaggaggactgaatactgactaaccggggtaggtgggtacatattttcgacggacacaggccctgggcacttgcaaaagaagtccgttctgcagaaccagcttacagaaccgaccttggagcgcaatgtcctggaaagaattcatgccccggtgctcgacacgtcgaaagaggaacaactcaaactcaggtaccagatgatgcccaccgaagccaacaaaagtaggtaccagtctcgtaaagtagaaaatcagaaagccataaccactgagcgactactgtcaggactacgactgtataactctgccacagatcagccagaatgctataagatcacctatccgaaaccattgtactccagtagcgtaccggcgaactactccgatccacagttcgctgtagctgtctgtaacaactatctgcatgagaactatccgacagtagcatcttatcagattactgacgagtacgatgcttacttggatatggtagacgggacagtcgcctgcctggatactgcaaccttctgccccgctaagcttagaagttacccgaaaaaacatgagtatagagccccgaatatccgcagtgcggttccatcagcgatgcagaacacgctacaaaatgtgctcattgccgcaactaaaagaaattgcaacgtcacgcagatgcgtgaactgccaacactggactcagcgacattcaatgtcgaatgctttcgaaaatatgcatgtaatgacgagtattgggaggagttcgctcggaagccaattaggattaccactgagtttgtcaccgcatatgtagctagactgaaaggccctaaggccgccgcactatttgcaaagacgtataatttggtcccattgcaagaagtgcctatggatagattcgtcatggacatgaaaagagacgtgaaagttacaccaggcacgaaacacacagaagaaagaccgaaagtacaagtgatacaagccgcagaacccctggcgactgcttacttatgcgggattcaccgggaattagtgcgtaggcttacggccgtcttgcttccaaacattcacacgctttttgacatgtcggcggaggattttgatgcaatcatagcagaacacttcaagcaaggcgacccggtactggagacggatatcgcatcattcgacaaaagccaagacgacgctatggcgttaaccggtctgatgatcttggaggacctgggtgtggatcaaccactactcgacttgatcgagtgcgcctttggagaaatatcatccacccatctacctacgggtactcgttttaaattcggggcgatgatgaaatccggaatgttcctcacactttttgtcaacacagttttgaatgtcgttatcgccagcagagtactagaggaacggcttaaaacgtccagatgtgcagcgttcattggcgacgacaacatcatacatggagtagtatctgacaaagaaatggctgagaggtgcgccacctggctcaacatggaggttaagatcatcgacgcagtcatcggtgagagaccaccttacttctgcggcggatttatcttgcaagattcggttacttccacagcgtgccgcgtggcggatcccctgaaaaggctgtttaagttgggtaaaccgctcccagccgacgacgagcaagacgaagacagaagacgcgctctgctagatgaaacaaaggcgtggtttagagtaggtataacaggcactttagcagtggccgtgacgacccggtatgaggtagacaatattacacctgtcctactggcattgagaacttttgcccagagcaaaagagcattccaagccatcagaggggaaataaagcatctctacggtggtcctaaatagtcagcatagtacatttcatctgactaatactacaacaccaccaccatgaagaccttaattcttgccgttgcattagtctactgcgccactgttcattgccaggactgtccttacgaacctgatccaccaaacacagttccaacttcctgtgaagctaaagaaggagaatgtattgatagcagctgtggcacctgcacgagagacatactatcagatggactgtgtgaaaataaaccaggaaaaacatgttgccgaatgtgtcagtatgtaattgaatgcagagtagaggccgcaggatggtttagaacattctatggaaagagattccagttccaggaacctggtacatacgtgttgggtcaaggaaccaagggcggcgactggaaggtgtccatcaccctggagaacctggatggaaccaagggggctgtgctgaccaagacaagactggaagtggctggagacatcattgacatcgctcaagctactgagaatcccatcactgtaaacggtggagctgaccctatcatcgccaacccgtacaccatcggcgaggtcaccatcgctgttgttgagatgccaggcttcaacatcaccgtcattgagttcttcaaactgatcgtgatcgacatcctcggaggaagatctgtaagaatcgccccagacacagcaaacaaaggaatgatctctggcctctgtggagatcttaaaatgatggaagatacagacttcacttcagatccagaacaactcgctattcagcctaagatcaaccaggagtttgacggttgtccactctatggaaatcctgatgacgttgcatactgcaaaggtcttctggagccgtacaaggacagctgccgcaaccccatcaacttctactactacaccatctcctgcgccttcgcccgctgtatgggtggagacgagcgagcctcacacgtgctgcttgactacagggagacgtgcgctgctcccgaaactagaggaacctgcgttttgtctggacatactttctacgatacatttgacaaagcaagataccaattccagggtccctgcaaggagattcttatggccgccgactgtttctggaacacttgggatgtgaaggtttcacacaggaatgttgactcttacactgaagtagagaaagtacgaatcaggaaacaatcgactgtagtagaactcattgttgatggaaaacagattctggttggaggagaagccgtgtccgtcccgtacagctctcagaacacttccatctactggcaagatggtgacatactgactacagccatcctacctgaagctctggtggtcaagttcaacttcaagcaactgctcgtcgtacatattagagatccattcgatggtaagacttgcggtatttgcggtaactacaaccaggatttcagtgatgattcttttgatgctgaaggagcctgtgatctgacccccaacccaccgggatgcaccgaagaacagaaacctgaagctgaacgactctgcaatagtctcttcgccggtcaaagtgatcttgatcagaaatgtaacgtgtgccacaagcctgaccgtgtcgaacgatgcatgtacgagtattgcctgaggggacaacagggtttctgtgaccacgcatgggagttcaagaaagaatgctacataaagcatggagacaccctagaagtaccagatgaatgcaaatagggcgcgcctatgttacgtgcaaaggtgattgtcaccccccgaaagaccatattgtgacacaccctcagtatcacgcccaaacatttacagccgcggtgtcaaaaaccgcgtggacgtggttaacatccctgctgggagaatcagccgtaattattataattggcttggtgctggctactattgtggccatgtacgtgctgaccaaccagaaacataattgaatacagcagcaattggcaagctgcttacatagaactcgcggcgattggcatgcgccttaaaatttttattttatttttcttttcttttccgaatcggattttgtttttaatatttcaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaagaagagctagggataacagggtaattgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagtacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacaccacatttccccgaaaagtgccacctgacgttagggataacagggtaat

SEQ ID NO: 60 - DNA, 11526 bp, synthetic construct
  source 1..11526: mol_type = other DNA
taatacgactcactataggattgacggcgtagtacacactattgaatcaaacagccgaccaattgcactaccatcacaatggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatacgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccaaggctgcttttgaggatgctcaggaggaagccagagcggagaagctccgagaagcacttccaccattagtggcagacaaaggcatcgaggcagccgcagaagttgtctgcgaagtggaggggctccaggcggacatcggagcagcattagttgaaaccccgcgcggtcacgtaaggataatacctcaagcaaatgaccgtatgatcggacagtatatcgttgtctcgccaaactctgtgctgaagaatgccaaactcgcaccagcgcacccgctagcagatcaggttaagatcataacacactccggaagatcaggaaggtacgcggtcgaaccatacgacgctaaagtactgatgccagcaggaggtgccgtaccatggccagaattcctagcactgagtgagagcgccacgttagtgtacaacgaaagagagtttgtgaaccgcaaactataccacattgccatgcatggccccgccaagaatacagaagaggagcagtacaaggttacaaaggcagagcttgcagaaacagagtacgtgtttgacgtggacaagaagcgttgcgttaagaaggaagaagcctcaggtctggtcctctcgggagaactgaccaaccctccctatcatgagctagctctggagggactgaagacccgacctgcggtcccgtacaaggtcgaaacaataggagtgataggcacaccggggtcgggcaagtcagctattatcaagtcaactgtcacggcacgagatcttgttaccagcggaaagaaagaaaattgtcgcgaaattgaggccgacgtgctaagactgaggggtatgcagattacgtcgaagacagtagattcggttatgctcaacggatgccacaaagccgtagaagtgctgtacgttgacgaagcgttcgcgtgccacgcaggagcactacttgccttgattgctatcgtcaggccccgcaagaaggtagtactatgcggagaccccatgcaatgcggattcttcaacatgatgcaactaaaggtacatttcaatcaccctgaaaaagacatatgcaccaagacattctacaagtatatctcccggcgttgcacacagccagttacagctattgtatcgacactgcattacgatggaaagatgaaaaccacgaacccgtgcaagaagaacattgaaatcgatattacaggggccacaaagccgaagccaggggatatcatcctgacatgtttccgcgggtgggttaagcaattgcaaatcgactatcccggacatgaagtaatgacagccgcggcctcacaagggctaaccagaaaaggagtgtatgccgtccggcaaaaagtcaatgaaaacccactgtacgcgatcacatcagagcatgtgaacgtgttgctcacccgcactgaggacaggctagtgtggaaaaccttgcagggcgacccatggattaagcagcccactaacatacctaaaggaaactttcaggctactatagaggactgggaagctgaacacaagggaataattgctgcaataaacagccccactccccgtgccaatccgttcagctgcaagaccaacgtttgctgggcgaaagcattggaaccgatactagccacggccggtatcgtacttaccggttgccagtggagcgaactgttcccacagtttgcggatgacaaaccacattcggccatttacgccttagacgtaatttgcattaagtttttcggcatggacttgacaagcggactgttttctaaacagagcatcccactaacgtaccatcccgccgattcagcgaggccggtagctcattgggacaacagcccaggaacccgcaagtatgggtacgatcacgccattgccgccgaactctcccgtagatttccggtgttccagctagctgggaagggcacacaacttgatttgcagacggggagaaccagagttatctctgcacagcataacctggtcccggtgaaccgcaatcttcctcacgccttagtccccgagtacaaggagaagcaacccggcccggtcaaaaaattcttgaaccagttcaaacaccactcagtacttgtggtatcagaggaaaaaattgaagctccccgtaagagaatcgaatggatcgccccgattggcatagccggtgcagataagaactacaacctggctttcgggtttccgccgcaggcacggtacgacctggtgttcatcaacattggaactaaatacagaaaccaccactttcagcagtgcgaagaccatgcggcgaccttaaaaaccctttcgcgttcggccctgaattgccttaacccaggaggcaccctcgtggtgaagtcctatggctacgccgaccgcaacagtgaggacgtagtcaccgctcttgccagaaagtttgtcagggtgtctgcagcgagaccagattgtgtctcaagcaatacagaaatgtacctgattttccgacaactagacaacagccgtacacggcaattcaccccgcaccatctgaattgcgtgatttcgtccgtgtatgagggtacaagagatggagttggagccgcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccccgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggatccatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttccactacgcaagcagagacgtagacgcaggagcaggaggactgaatactgactaaccggggtaggtgggtacatattttcgacggacacaggccctgggcacttgcaaaagaagtccgttctgcagaaccagcttacagaaccgaccttggagcgcaatgtcctggaaagaattcatgccccggtgctcgacacgtcgaaagaggaacaactcaaactcaggtaccagatgatgcccaccgaagccaacaaaagtaggtaccagtctcgtaaagtagaaaatcagaaagccataaccactgagcgactactgtcaggactacgactgtataactctgccacagatcagccagaatgctataagatcacctatccgaaaccattgtactccagtagcgtaccggcgaactactccgatccacagttcgctgtagctgtctgtaacaactatctgcatgagaactatccgacagtagcatcttatcagattactgacgagtacgatgcttacttggatatggtagacgggacagtcgcctgcctggatactgcaaccttctgccccgctaagcttagaagttacccgaaaaaacatgagtatagagccccgaatatccgcagtgcggttccatcagcgatgcagaacacgctacaaaatgtgctcattgccgcaactaaaagaaattgcaacgtcacgcagatgcgtgaactgccaacactggactcagcgacattcaatgtcgaatgctttcgaaaatatgcatgtaatgacgagtattgggaggagttcgctcggaagccaattaggattaccactgagtttgtcaccgcatatgtagctagactgaaaggccctaaggccgccgcactatttgcaaagacgtataatttggtcccattgcaagaagtgcctatggatagattcgtcatggacatgaaaagagacgtgaaagttacaccaggcacgaaacacacagaagaaagaccgaaagtacaagtgatacaagccgcagaacccctggcgactgcttacttatgcgggattcaccgggaattagtgcgtaggcttacggccgtcttgcttccaaacattcacacgctttttgacatgtcggcggaggattttgatgcaatcatagcagaacacttcaagcaaggcgacccggtactggagacggatatcgcatcattcgacaaaagccaagacgacgctatggcgttaaccggtctgatgatcttggaggacctgggtgtggatcaaccactactcgacttgatcgagtgcgcctttggagaaatatcatccacccatctacctacgggtactcgttttaaattcggggcgatgatgaaatccggaatgttcctcacactttttgtcaacacagttttgaatgtcgttatcgccagcagagtactagaggaacggcttaaaacgtccagatgtgcagcgttcattggcgacgacaacatcatacatggagtagtatctgacaaagaaatggctgagaggtgcgccacctggctcaacatggaggttaagatcatcgacgcagtcatcggtgagagaccaccttacttctgcggcggatttatcttgcaagattcggttacttccacagcgtgccgcgtggcggatcccctgaaaaggctgtttaagttgggtaaaccgctcccagccgacgacgagcaagacgaagacagaagacgcgctctgctagatgaaacaaaggcgtggtttagagtaggtataacaggcactttagcagtggccgtgacgacccggtatgaggtagacaatattacacctgtcctactggcattgagaacttttgcccagagcaaaagagcattccaagccatcagaggggaaataaagcatctctacggtggtcctaaatagtcagcatagtacatttcatctgactaatactacaacaccaccaccatgaagaccttaattcttgccgttgcattagtctactgcgccactgttcattgccaggactgtccttacgaacctgatccaccaaacacagttccaacttcctgtgaagctaaagaaggagaatgtattgatagcagctgtggcacctgcacgagagacatactatcagatggactgtgtgaaaataaaccaggaaaaacatgttgccgaatgtgtcagtatgtaattgaatgcagagtagaggccgcaggatggtttagaacattctatggaaagagattccagttccaggaacctggtacatacgtgttgggtcaaggaaccaagggcggcgactggaaggtgtccatcaccctggagaacctggatggaaccaagggggctgtgctgaccaagacaagactggaagtggctggagacatcattgacatcgctcaagctactgagaatcccatcactgtaaacggtggagctgaccctatcatcgccaacccgtacaccatcggcgaggtcaccatcgctgttgttgagatgccaggcttcaacatcaccgtcattgagttcttcaaactgatcgtgatcgacatcctcggaggaagatctgtaagaatcgccccagacacagcaaacaaaggaatgatctctggcctctgtggagatcttaaaatgatggaagatacagacttcacttcagatccagaacaactcgctattcagcctaagatcaaccaggagtttgacggttgtccactctatggaaatcctgatgacgttgcatactgcaaaggtcttctggagccgtacaaggacagctgccgcaaccccatcaacttctactactacaccatctcctgcgccttcgcccgctgtatgggtggagacgagcgagcctcacacgtgctgcttgactacagggagacgtgcgctgctcccgaaactagaggaacctgcgttttgtctggacatactttctacgatacatttgacaaagcaagataccaattccagggtccctgcaaggagattcttatggccgccgactgtttctggaacacttgggatgtgaaggtttcacacaggaatgttgactcttacactgaagtagagaaagtacgaatcaggaaacaatcgactgtagtagaactcattgttgatggaaaacagattctggttggaggagaagccgtgtccgtcccgtacagctctcagaacacttccatctactggcaagatggtgacatactgactacagccatcctacctgaagctctggtggtcaagttcaacttcaagcaactgctcgtcgtacatattagagatccattcgatggtaagacttgcggtatttgcggtaactacaaccaggatttcagtgatgattcttttgatgctgaaggagcctgtgatctgacccccaacccaccgggatgcaccgaagaacagaaacctgaagctgaacgactctgcaatagtctcttcgccggtcaaagtgatcttgatcagaaatgtaacgtgtgccacaagcctgaccgtgtcgaacgatgcatgtacgagtattgcctgaggggacaacagggtttctgtgaccacgcatgggagttcaagaaagaatgctacataaagcatggagacaccctagaagtaccagatgaatgcaaatagggcgcgcctatgttacgtgcaaaggtgattgtcaccccccgaaagaccatattgtgacacaccctcagtatcacgcccaaacatttacagccgcggtgtcaaaaaccgcgtggacgtggttaacatccctgctgggagaatcagccgtaattattataattggcttggtgctggctactattgtggccatgtacgtgctgaccaaccagaaacataattgaatacagcagcaattggcaagctgcttacatagaactcgcggcgattggcatgcgccttaaaatttttattttatttttcttttcttttccgaatcggattttgtttttaatatttcaaaaaaaaaaaaaaaaaaaaaaaaaaaaaaaaaaaaaaaaaagaagagctagggataacagggtaattgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaattatatatgagtaaacttggtctgacagtacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacaccacatttccccgaaaagtgccacctgacgttagggataacagggtaat

SEQ ID NO: 61 - RNA, 9742 nt, synthetic construct
  source 1..9742: mol_type = other RNA
ggattgacggcgtagtacacactattgaatcaaacagccgaccaattgcactaccatcacaatggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatacgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccaaggctgcttttgaggatgctcaggaggaagccagagcggagaagctccgagaagcacttccaccattagtggcagacaaaggcatcgaggcagccgcagaagttgtctgcgaagtggaggggctccaggcggacatcggagcagcattagttgaaaccccgcgcggtcacgtaaggataatacctcaagcaaatgaccgtatgatcggacagtatatcgttgtctcgccaaactctgtgctgaagaatgccaaactcgcaccagcgcacccgctagcagatcaggttaagatcataacacactccggaagatcaggaaggtacgcggtcgaaccatacgacgctaaagtactgatgccagcaggaggtgccgtaccatggccagaattcctagcactgagtgagagcgccacgttagtgtacaacgaaagagagtttgtgaaccgcaaactataccacattgccatgcatggccccgccaagaatacagaagaggagcagtacaaggttacaaaggcagagcttgcagaaacagagtacgtgtttgacgtggacaagaagcgttgcgttaagaaggaagaagcctcaggtctggtcctctcgggagaactgaccaaccctccctatcatgagctagctctggagggactgaagacccgacctgcggtcccgtacaaggtcgaaacaataggagtgataggcacaccggggtcgggcaagtcagctattatcaagtcaactgtcacggcacgagatcttgttaccagcggaaagaaagaaaattgtcgcgaaattgaggccgacgtgctaagactgaggggtatgcagattacgtcgaagacagtagattcggttatgctcaacggatgccacaaagccgtagaagtgctgtacgttgacgaagcgttcgcgtgccacgcaggagcactacttgccttgattgctatcgtcaggccccgcaagaaggtagtactatgcggagaccccatgcaatgcggattcttcaacatgatgcaactaaaggtacatttcaatcaccctgaaaaagacatatgcaccaagacattctacaagtatatctcccggcgttgcacacagccagttacagctattgtatcgacactgcattacgatggaaagatgaaaaccacgaacccgtgcaagaagaacattgaaatcgatattacaggggccacaaagccgaagccaggggatatcatcctgacatgtttccgcgggtgggttaagcaattgcaaatcgactatcccggacatgaagtaatgacagccgcggcctcacaagggctaaccagaaaaggagtgtatgccgtccggcaaaaagtcaatgaaaacccactgtacgcgatcacatcagagcatgtgaacgtgttgctcacccgcactgaggacaggctagtgtggaaaaccttgcagggcgacccatggattaagcagcccactaacatacctaaaggaaactttcaggctactatagaggactgggaagctgaacacaagggaataattgctgcaataaacagccccactccccgtgccaatccgttcagctgcaagaccaacgtttgctgggcgaaagcattggaaccgatactagccacggccggtatcgtacttaccggttgccagtggagcgaactgttcccacagtttgcggatgacaaaccacattcggccatttacgccttagacgtaatttgcattaagtttttcggcatggacttgacaagcggactgttttctaaacagagcatcccactaacgtaccatcccgccgattcagcgaggccggtagctcattgggacaacagcccaggaacccgcaagtatgggtacgatcacgccattgccgccgaactctcccgtagatttccggtgttccagctagctgggaagggcacacaacttgatttgcagacggggagaaccagagttatctctgcacagcataacctggtcccggtgaaccgcaatcttcctcacgccttagtccccgagtacaaggagaagcaacccggcccggtcaaaaaattcttgaaccagttcaaacaccactcagtacttgtggtatcagaggaaaaaattgaagctccccgtaagagaatcgaatggatcgccccgattggcatagccggtgcagataagaactacaacctggctttcgggtttccgccgcaggcacggtacgacctggtgttcatcaacattggaactaaatacagaaaccaccactttcagcagtgcgaagaccatgcggcgaccttaaaaaccctttcgcgttcggccctgaattgccttaacccaggaggcaccctcgtggtgaagtcctatggctacgccgaccgcaacagtgaggacgtagtcaccgctcttgccagaaagtttgtcagggtgtctgcagcgagaccagattgtgtctcaagcaatacagaaatgtacctgattttccgacaactagacaacagccgtacacggcaattcaccccgcaccatctgaattgcgtgatttcgtccgtgtatgagggtacaagagatggagttggagccgcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccccgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggatccatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttccactacgcaagcagagacgtagacgcaggagcaggaggactgaatactgactaaccggggtaggtgggtacatattttcgacggacacaggccctgggcacttgcaaaagaagtccgttctgcagaaccagcttacagaaccgaccttggagcgcaatgtcctggaaagaattcatgccccggtgctcgacacgtcgaaagaggaacaactcaaactcaggtaccagatgatgcccaccgaagccaacaaaagtaggtaccagtctcgtaaagtagaaaatcagaaagccataaccactgagcgactactgtcaggactacgactgtataactctgccacagatcagccagaatgctataagatcacctatccgaaaccattgtactccagtagcgtaccggcgaactactccgatccacagttcgctgtagctgtctgtaacaactatctgcatgagaactatccgacagtagcatcttatcagattactgacgagtacgatgcttacttggatatggtagacgggacagtcgcctgcctggatactgcaaccttctgccccgctaagcttagaagttacccgaaaaaacatgagtatagagccccgaatatccgcagtgcggttccatcagcgatgcagaacacgctacaaaatgtgctcattgccgcaactaaaagaaattgcaacgtcacgcagatgcgtgaactgccaacactggactcagcgacattcaatgtcgaatgctttcgaaaatatgcatgtaatgacgagtattgggaggagttcgctcggaagccaattaggattaccactgagtttgtcaccgcatatgtagctagactgaaaggccctaaggccgccgcactatttgcaaagacgtataatttggtcccattgcaagaagtgcctatggatagattcgtcatggacatgaaaagagacgtgaaagttacaccaggcacgaaacacacagaagaaagaccgaaagtacaagtgatacaagccgcagaacccctggcgactgcttacttatgcgggattcaccgggaattagtgcgtaggcttacggccgtcttgcttccaaacattcacacgctttttgacatgtcggcggaggattttgatgcaatcatagcagaacacttcaagcaaggcgacccggtactggagacggatatcgcatcattcgacaaaagccaagacgacgctatggcgttaaccggtctgatgatcttggaggacctgggtgtggatcaaccactactcgacttgatcgagtgcgcctttggagaaatatcatccacccatctacctacgggtactcgttttaaattcggggcgatgatgaaatccggaatgttcctcacactttttgtcaacacagttttgaatgtcgttatcgccagcagagtactagaggaacggcttaaaacgtccagatgtgcagcgttcattggcgacgacaacatcatacatggagtagtatctgacaaagaaatggctgagaggtgcgccacctggctcaacatggaggttaagatcatcgacgcagtcatcggtgagagaccaccttacttctgcggcggatttatcttgcaagattcggttacttccacagcgtgccgcgtggcggatcccctgaaaaggctgtttaagttgggtaaaccgctcccagccgacgacgagcaagacgaagacagaagacgcgctctgctagatgaaacaaaggcgtggtttagagtaggtataacaggcactttagcagtggccgtgacgacccggtatgaggtagacaatattacacctgtcctactggcattgagaacttttgcccagagcaaaagagcattccaagccatcagaggggaaataaagcatctctacggtggtcctaaatagtcagcatagtacatttcatctgactaatactacaacaccaccaccatgaagaccttaattcttgccgttgcattagtctactgcgccactgttcattgccaggactgtccttacgaacctgatccaccaaacacagttccaacttcctgtgaagctaaagaaggagaatgtattgatagcagctgtggcacctgcacgagagacatactatcagatggactgtgtgaaaataaaccaggaaaaacatgttgccgaatgtgtcagtatgtaattgaatgcagagtagaggccgcaggatggtttagaacattctatggaaagagattccagttccaggaacctggtacatacgtgttgggtcaaggaaccaagggcggcgactggaaggtgtccatcaccctggagaacctggatggaaccaagggggctgtgctgaccaagacaagactggaagtggctggagacatcattgacatcgctcaagctactgagaatcccatcactgtaaacggtggagctgaccctatcatcgccaacccgtacaccatcggcgaggtcaccatcgctgttgttgagatgccaggcttcaacatcaccgtcattgagttcttcaaactgatcgtgatcgacatcctcggaggaagatctgtaagaatcgccccagacacagcaaacaaaggaatgatctctggcctctgtggagatcttaaaatgatggaagatacagacttcacttcagatccagaacaactcgctattcagcctaagatcaaccaggagtttgacggttgtccactctatggaaatcctgatgacgttgcatactgcaaaggtcttctggagccgtacaaggacagctgccgcaaccccatcaacttctactactacaccatctcctgcgccttcgcccgctgtatgggtggagacgagcgagcctcacacgtgctgcttgactacagggagacgtgcgctgctcccgaaactagaggaacctgcgttttgtctggacatactttctacgatacatttgacaaagcaagataccaattccagggtccctgcaaggagattcttatggccgccgactgtttctggaacacttgggatgtgaaggtttcacacaggaatgttgactcttacactgaagtagagaaagtacgaatcaggaaacaatcgactgtagtagaactcattgttgatggaaaacagattctggttggaggagaagccgtgtccgtcccgtacagctctcagaacacttccatctactggcaagatggtgacatactgactacagccatcctacctgaagctctggtggtcaagttcaacttcaagcaactgctcgtcgtacatattagagatccattcgatggtaagacttgcggtatttgcggtaactacaaccaggatttcagtgatgattcttttgatgctgaaggagcctgtgatctgacccccaacccaccgggatgcaccgaagaacagaaacctgaagctgaacgactctgcaatagtctcttcgccggtcaaagtgatcttgatcagaaatgtaacgtgtgccacaagcctgaccgtgtcgaacgatgcatgtacgagtattgcctgaggggacaacagggtttctgtgaccacgcatgggagttcaagaaagaatgctacataaagcatggagacaccctagaagtaccagatgaatgcaaatagggcgcgcctatgttacgtgcaaaggtgattgtcaccccccgaaagaccatattgtgacacaccctcagtatcacgcccaaacatttacagccgcggtgtcaaaaaccgcgtggacgtggttaacatccctgctgggagaatcagccgtaattattataattggcttggtgctggctactattgtggccatgtacgtgctgaccaaccagaaacataattgaatacagcagcaattggcaagctgcttacatagaactcgcggcgattggcatgcgccttaaaatttttattttatttttcttttcttttccgaatcggattttgtttttaatatttcaaaaaaaaaaaaaaaaaaaaaaaaaaaagcatatgactaaaaaaaaaaaaaaaaaaaaaaaaaaaaaaaaaaaaaaaaaaaaaaaaaaaaaaaaa

SEQ ID NO: 62 - DNA, 6904 bp, synthetic construct
  source 1..6904: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattttaagttgcattataggagaacccaagcttggtaccgagctcggatccgccaccatgaagaccttaattcttgccgttgcattagtctactgcgccactgttcattgccaggactgtccttacgaacctgatccaccaaacacagttccaacttcctgtgaagctaaagaaggagaatgtattgatagcagctgtggcacctgcacgagagacatactatcagatggactgtgtgaaaataaaccaggaaaaacatgttgccgaatgtgtcagtatgtaattgaatgcagagtagaggccgcaggatggtttagaacattctatggaaagagattccagttccaggaacctggtacatacgtgttgggtcaaggaaccaagggcggcgactggaaggtgtccatcaccctggagaacctggatggaaccaagggggctgtgctgaccaagacaagactggaagtggctggagacatcattgacatcgctcaagctactgagaatcccatcactgtaaacggtggagctgaccctatcatcgccaacccgtacaccatcggcgaggtcaccatcgctgttgttgagatgccaggcttcaacatcaccgtcattgagttcttcaaactgatcgtgatcgacatcctcggaggaagatctgtaagaatcgccccagacacagcaaacaaaggaatgatctctggcctctgtggagatcttaaaatgatggaagatacagacttcacttcagatccagaacaactcgctattcagcctaagatcaaccaggagtttgacggttgtccactctatggaaatcctgatgacgttgcatactgcaaaggtcttctggagccgtacaaggacagctgccgcaaccccatcaacttctactactacaccatctcctgcgccttcgcccgctgtatgggtggagacgagcgagcctcacacgtgctgcttgactacagggagacgtgcgctgctcccgaaactagaggaacctgcgttttgtctggacatactttctacgatacatttgacaaagcaagataccaattccagggtccctgcaaggagattcttatggccgccgactgtttctggaacacttgggatgtgaaggtttcacacaggaatgttgactcttacactgaagtagagaaagtacgaatcaggaaacaatcgactgtagtagaactcattgttgatggaaaacagattctggttggaggagaagccgtgtccgtcccgtacagctctcagaacacttccatctactggcaagatggtgacatactgactacagccatcctacctgaagctctggtggtcaagttcaacttcaagcaactgctcgtcgtacatattagagatccattcgatggtaagacttgcggtatttgcggtaactacaaccaggatttcagtgatgattcttttgatgctgaaggagcctgtgatctgacccccaacccaccgggatgcaccgaagaacagaaacctgaagctgaacgactctgcaatagtctcttcgccggtcaaagtgatcttgatcagaaatgtaacgtgtgccacaagcctgaccgtgtcgaacgatgcatgtacgagtattgcctgaggggacaacagggtttctgtgaccacgcatgggagttcaagaaagaatgctacataaagcatggagacaccctagaagtaccagatgaatgcaaataggcggccgcaataaaatatctttattttcattacatctgtgtgttggttttttgtgtgtctagaaataattcttactgtcatgccaagtaagatgcttttctgtgctgcaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 63 - DNA, 6906 bp, synthetic construct
  source 1..6906: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccgccaccatgaagaccttaattcttgccgttgcattagtctactgcgccactgttcattgccaggactgtccttacgaacctgatccaccaaacacagttccaacttcctgtgaagctaaagaaggagaatgtattgatagcagctgtggcacctgcacgagagacatactatcagatggactgtgtgaaaataaaccaggaaaaacatgttgccgaatgtgtcagtatgtaattgaatgcagagtagaggccgcaggatggtttagaacattctatggaaagagattccagttccaggaacctggtacatacgtgttgggtcaaggaaccaagggcggcgactggaaggtgtccatcaccctggagaacctggatggaaccaagggggctgtgctgaccaagacaagactggaagtggctggagacatcattgacatcgctcaagctactgagaatcccatcactgtaaacggtggagctgaccctatcatcgccaacccgtacaccatcggcgaggtcaccatcgctgttgttgagatgccaggcttcaacatcaccgtcattgagttcttcaaactgatcgtgatcgacatcctcggaggaagatctgtaagaatcgccccagacacagcaaacaaaggaatgatctctggcctctgtggagatcttaaaatgatggaagatacagacttcacttcagatccagaacaactcgctattcagcctaagatcaaccaggagtttgacggttgtccactctatggaaatcctgatgacgttgcatactgcaaaggtcttctggagccgtacaaggacagctgccgcaaccccatcaacttctactactacaccatctcctgcgccttcgcccgctgtatgggtggagacgagcgagcctcacacgtgctgcttgactacagggagacgtgcgctgctcccgaaactagaggaacctgcgttttgtctggacatactttctacgatacatttgacaaagcaagataccaattccagggtccctgcaaggagattcttatggccgccgactgtttctggaacacttgggatgtgaaggtttcacacaggaatgttgactcttacactgaagtagagaaagtacgaatcaggaaacaatcgactgtagtagaactcattgttgatggaaaacagattctggttggaggagaagccgtgtccgtcccgtacagctctcagaacacttccatctactggcaagatggtgacatactgactacagccatcctacctgaagctctggtggtcaagttcaacttcaagcaactgctcgtcgtacatattagagatccattcgatggtaagacttgcggtatttgcggtaactacaaccaggatttcagtgatgattcttttgatgctgaaggagcctgtgatctgacccccaacccaccgggatgcaccgaagaacagaaacctgaagctgaacgactctgcaatagtctcttcgccggtcaaagtgatcttgatcagaaatgtaacgtgtgccacaagcctgaccgtgtcgaacgatgcatgtacgagtattgcctgaggggacaacagggtttctgtgaccacgcatgggagttcaagaaagaatgctacataaagcatggagacaccctagaagtaccagatgaatgcaaataggcggccgcaataaaatatctttattttcattacatctgtgtgttggttttttgtgtgtctagaaataattcttactgtcatgccaagtaagatgcttttctgtgctgcaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtca

SEQ ID NO: 64 - RNA, 1703 nt, synthetic construct
  source 1..1703: mol_type = other RNA
ggagaacccaagcttggtaccgagctcggatccgccaccatgaagaccttaattcttgccgttgcattagtctactgcgccactgttcattgccaggactgtccttacgaacctgatccaccaaacacagttccaacttcctgtgaagctaaagaaggagaatgtattgatagcagctgtggcacctgcacgagagacatactatcagatggactgtgtgaaaataaaccaggaaaaacatgttgccgaatgtgtcagtatgtaattgaatgcagagtagaggccgcaggatggtttagaacattctatggaaagagattccagttccaggaacctggtacatacgtgttgggtcaaggaaccaagggcggcgactggaaggtgtccatcaccctggagaacctggatggaaccaagggggctgtgctgaccaagacaagactggaagtggctggagacatcattgacatcgctcaagctactgagaatcccatcactgtaaacggtggagctgaccctatcatcgccaacccgtacaccatcggcgaggtcaccatcgctgttgttgagatgccaggcttcaacatcaccgtcattgagttcttcaaactgatcgtgatcgacatcctcggaggaagatctgtaagaatcgccccagacacagcaaacaaaggaatgatctctggcctctgtggagatcttaaaatgatggaagatacagacttcacttcagatccagaacaactcgctattcagcctaagatcaaccaggagtttgacggttgtccactctatggaaatcctgatgacgttgcatactgcaaaggtcttctggagccgtacaaggacagctgccgcaaccccatcaacttctactactacaccatctcctgcgccttcgcccgctgtatgggtggagacgagcgagcctcacacgtgctgcttgactacagggagacgtgcgctgctcccgaaactagaggaacctgcgttttgtctggacatactttctacgatacatttgacaaagcaagataccaattccagggtccctgcaaggagattcttatggccgccgactgtttctggaacacttgggatgtgaaggtttcacacaggaatgttgactcttacactgaagtagagaaagtacgaatcaggaaacaatcgactgtagtagaactcattgttgatggaaaacagattctggttggaggagaagccgtgtccgtcccgtacagctctcagaacacttccatctactggcaagatggtgacatactgactacagccatcctacctgaagctctggtggtcaagttcaacttcaagcaactgctcgtcgtacatattagagatccattcgatggtaagacttgcggtatttgcggtaactacaaccaggatttcagtgatgattcttttgatgctgaaggagcctgtgatctgacccccaacccaccgggatgcaccgaagaacagaaacctgaagctgaacgactctgcaatagtctcttcgccggtcaaagtgatcttgatcagaaatgtaacgtgtgccacaagcctgaccgtgtcgaacgatgcatgtacgagtattgcctgaggggacaacagggtttctgtgaccacgcatgggagttcaagaaagaatgctacataaagcatggagacaccctagaagtaccagatgaatgcaaataggc

SEQ ID NO: 65 - DNA, 404 bp, synthetic construct
  source 1..404: mol_type = other DNA
aaatttaaatttaaataatacgactcactataggctcgttatagccaacataactgcgcagctgaaccgtcacgaatgccacctatagcccaatacaacacaatcgtgcaatgctgcaaatggtcctcgttgcggtcagacgtatgggaactctttgctacagacccattcggttcctactgcacgatgattccgacaaagaatcggtagactaactttacgatacacgtacccggcgtaatattgtcgcgaactccataggcttacgccagtcatcttacgtagttgtgggagcactagtccgtgttatccgctaccgtcatacgaccctctaattcgctacataaggtgcatgcgtacatggtaatcctgggatggcatggatgaactgtacaactgagggg

SEQ ID NO: 66 - DNA, 404 bp, synthetic construct
  source 1..404: mol_type = other DNA
aaatttaaatttaaagtttaagttgcattataggctcgttatagccaacataactgcgcagctgaaccgtcacgaatgccacctatagcccaatacaacacaatcgtgcaatgctgcaaatggtcctcgttgcggtcagacgtatgggaactctttgctacagacccattcggttcctactgcacgatgattccgacaaagaatcggtagactaactttacgatacacgtacccggcgtaatattgtcgcgaactccataggcttacgccagtcatcttacgtagttgtgggagcactagtccgtgttatccgctaccgtcatacgaccctctaattcgctacataaggtgcatgcgtacatggtaatcctgggatggcatggatgaactgtacaactgagggg

SEQ ID NO: 67 - RNA, 372 nt, synthetic construct
  source 1..372: mol_type = other RNA
ggctcgttatagccaacataactgcgcagctgaaccgtcacgaatgccacctatagcccaatacaacacaatcgtgcaatgctgcaaatggtcctcgttgcggtcagacgtatgggaactctttgctacagacccattcggttcctactgcacgatgattccgacaaagaatcggtagactaactttacgatacacgtacccggcgtaatattgtcgcgaactccataggcttacgccagtcatcttacgtagttgtgggagcactagtccgtgttatccgctaccgtcatacgaccctctaattcgctacataaggtgcatgcgtacatggtaatcctgggatggcatggatgaactgtacaactgagggg

SEQ ID NO: 68 - DNA, 23 bp, synthetic construct
  source 1..23: mol_type = other DNA
  misc_feature 1: note = 5Bios
ggtgtgtactacgccgtcaatcc

SEQ ID NO: 69 - DNA, 19 bp, synthetic construct
  source 1..19: mol_type = other DNA
taatacgactcactatagg

SEQ ID NO: 70 - DNA, 19 bp, synthetic construct
  source 1..19: mol_type = other DNA
atttaggtgacactataga

SEQ ID NO: 71 - DNA, 19 bp, synthetic construct
  source 1..19: mol_type = other DNA
taatacgactcactatagg

SEQ ID NO: 72 - DNA, 19 bp, synthetic construct
  source 1..19: mol_type = other DNA
taatacgactcactatagt

SEQ ID NO: 73 - DNA, 19 bp, synthetic construct
  source 1..19: mol_type = other DNA
taatacgactcactataag

SEQ ID NO: 74 - DNA, 19 bp, synthetic construct
  source 1..19: mol_type = other DNA
taatacgactcactataat

SEQ ID NO: 75 - protein, 873 aa, synthetic construct
MAKRLQRCQDVNQVCEIYNSKGGIGELELRFDKLPQNLFAGVFDKLKPDGEIQTTMRVSNRDGVAREITFGGGVKTNEIFVKKQNICVFDVVDIFSYKVAVSTEETVVEKPTMETTAGVRFKIRLSVEDVVKDWRIDLTAVKTAELGKIAQHTASIVQRTFPDNLLKLTGAEVAKLAADSYELELEYTGKSPATNEKVNVAAKYAVELLSSVRNANSTAAASFGESVSDLCRVAKIIHTHEYANVVCRTPSFKMLLPQVVSLTKSSYYGGLYPPENLWLAGKTDGVRALVVCEDGVAKVITAESVDITHGVCSATTILDCELNVDAKILYVFDVIISNNTQVYTQPFSTRITTDISDIKIDGYKIEMKPFVKVVKADEATFKSAYKAPHNEGLIMIEDGAAYAATKTYKWKPLSHNTIDFLIKACPKQLINVDPYKPRAGYKLWLLFTTISLDQQRELGIEFIPAWKILFTDINMFGSRVPIQFQPAINPLAYVCYLPEDVNVNDGDIVEMRAVDGYDTIPKWELVRSRNDRKNEPGFYGNNYKIASDIYLNYIDVFHFEDLYKYNPGYFEKNKSDIYVAPNKYRRYLIKSLFGRYLRDAKWVIDAAAGRGADLHLYKAECVEHLLAIDIDPTAISELVRRRNEITGYNKSHRGGRNMHSHRGQSHCAKSTSLHALVADLRENPDVLIPKIIQSRPHERCYDAIVINFAIHYLCDTDEHIRDFLITVSRLLAPNGVFIFTTMDGESIVKLLADHKVRPGEAWTIHTGDVNSPDSTVPKYSIRRLYDSDKLTKTGQQIEVLLPMSGEMKAEPLCNIKNIISMARKMGLDLVESANFSVLYEAYARDYPDIYARMTPDDKLYNDLHTYAVFKRKK

SEQ ID NO: 76 - protein, 890 aa, synthetic construct
MRRVFNSAKKQQRCDSVEQVCEFYNADNKTNELELRFDKLNRELFVVLFDKLKPDGEITTTMRVSNADGMAREITFGGGVKTGEMFVKKQNICVFDVVDVFSYKVAVSSEDEIKDKPKMDTNASVRFKIRLSCDTLIPDWRIDLTAVKVADLGKIAQHTSTVVLQTFPENLLRMKGAEVAALATNSYELELEYIGKSAASKEKVLAAAEYAMELLTNSRNAISPAAATLGESVSDICRIAKLIHPAEYANVICRTPSFKNLLPQVISLTKSSYYGGIYPPVDMYIAGKTDGVRALVLCENGVAKIITATTVDTTTVGNTPITILDCELSTSGHNGATDNKHLYIFDVIMNRGVHSHREGFNKRIDIDLSDLTPAGYTLELKPFTKLVDAASVNETTFKSVFKPPHNEGLVLVESGPPYALTKTYKWKPITHNTIDFLIKACPKQLINVDPFKPRNGHDLWLLFTTISLDQQRELGIDLIPAWKLLFTDVNLFGNKIPIQFMPAINPLAYICYLPSSTGVNDGDIVEMRAVDGFDGIPTWELVRTRPDRKDERGFYGNNYKIASDIYLNYIDVFNFDDLWKYNPGYFEKNKSDIYIAPNKYRRYLIKSLFNKYIKNAKWVIDAAAGRGADLHLYKQECVENLLAIDIDPTAISELIRRRNEITGWQQRGRGGNTRHNARHNTHCASSTSLHALVADLRTEPNMLIPKIIQSRPPERGYDAIVINFAIHYLCETDDYIRNFLITVSRLLAPDGVFIFTTMDGEAIVNLLAEHKVAPGASWVVHTDGNANATDANVVKYSIKRLYDSDKLTKTGQKIAVLLPMSGEMREEPLCNIKNIVSMARKMGLDLVESANFSVLYGAYAKDYPEIYAKLTPDDKLYNDLHAFAVFKRKK

SEQ ID NO: 77 - protein, 894 aa, synthetic construct
MRRVSNSAKKQQRCDSVEQVCEFYNADNKTNELELRFDKLNRELFVALFDKLKPDGEITTTMRVSNADGMAREITFGGGVKTGEMFVKKQNICVFDVVDVFSYKVAVSSEDEVKDKPKMDTNASVRFKIRLSCDTLIPDWRIDLTAVKVADLGKIAQHTSTVVLQTFPENLLRMKGAEVAALATNSYELELEYIGKSTAGKEKVLKAAEYAIELLTNLRNAVSPVAATLGESVSDICRIAKLIHPAEYANVICRTPSFKNLLPQVISLTKSSYYGGIYPPVDMYITGKTDGVRALVLCENGVAKIITATTVDTTTVGDTPITILDCELSTSGHNGATDNKHLYVFDVIMNRGAHSHRDGFNKRIDIDLSDLTPAGYTLELKPFTKVADAASVNETTFKSVFKPPHNEGLVLVESGPPYALTKTYKWKPITHNTIDFLIKACPKQLINVDPFKPRNGHDLWLLFTTISLDQQRELGIDLIPAWKLLFTDVNLFGNKIPIQFMPAINPLAYICYLPSSTGVNDGDIVEMRAVDGFDGIPTWELVRTRPDRKDERGFYGNNYKIASDIYLNYIDVFNFDDLWKYNPGYFEKNKSDIYIAPNKYRRYLIKSLFNKYIKNAKWVIDAAAGRGADLHLYKQECVENLLAIDIDPTAISELIRRRNEITGWQQRGRGGNMHHNSRHNTRHNTHCASSTSLHALVADLRTEPNMLIPKIIQSRPPERGYDALVINFAIHYLCETDDYIRNFLITVSRLLAHDGVFIFTTMDGEAIVNLLTEHKVAPGASWVVHTDGNTNATDANVVKYSIKRLYDSDKLTKTGQKIAVLLPMSGEMREEPLCNIKNIVSMARKMGLDLVESANFSVLYGAYAKDYPEIYAKLTPDDKLYNDLHAFAVFKRKK

SEQ ID NO: 78 - protein, 901 aa, synthetic construct
LEYQYYKYILVNIMSRRLQRCRDVDQVCEYYNAKGAIGELELRFDKLTPDLFAHVFDKLKPDGEISTTMRVSNSDGTAREITFGGGVKTGETFVRKQNICVFDVVDIFSYKVAVSTEETLVDKPAMEKDASVRFKIRMSVEGAVPNWRIDLTAVKTAELGKIAQHTASLVLQTFPPNLLKMSGAEVAKLANNSYELELEYIGKTPATKERVDAAAKYAVDLLAGIKNANSAVGAVLGESISDICRVAKVIHTPDYATVVCRNPSFKMLLPQVISLTKSSYYGGIYPPEGMYVAGKTDGVRALVLCEDGVAKVITAESVDITTGTCTGTTILDCELSTGKSGATLHVFDIIMHNSKPIHSQPFSTRIATDISDVKIPEYKIAIKPFVKIQATALEAAFKEVYKAPHNEGLILIMDGNDYAMTKTYKWKPLSHNTIDFLIKACPKQLLNIDPYKPRPGHKLWLLFTTISLDQQRELGIEFIPAWKMLFTDINLFGSRVPIQFMPAINPLAYICYLPEAATCANGDAINDGDIVEMRAVDGFDTVPKWEPIRVRSDRKDEPGFYGNNYKIASDIYLNYIDIFQFEDLYKYNPGYFEKNKSDIYVAPNKYRRFLIKNIFSKYLKNAKWVIDAAAGRGADLHLYKAECVENLLAIDIDPTAISELVRRRNEITGYNRGHRGHRGGSMRAHMGASHHGAQNCAKSTTLHALVADLRTDPDVLIPKIIQSRPPERGYDAIVINFAIHYLCDTDEHIRDFLITVSRLLAPNGIFMFTTMDGESIVKLLETHKVKSGESWTVHTGADDPEAGVIKYSIRRLYDSDKLTKTGQQIAVLLPMSGEMKTEPLCNIKNIISIARKMGLDLVESADFSVMYDAFARAYPEISARLTPDDKLYNDLHSYAVFKRKK

SEQ ID NO: 79 - protein, 1217 aa, synthetic construct
MQKSNIQSKIGNDNVKQIEDLFKKINKDSEFEFIFFSKKDKHLTFEKYRTLVKFISKRASSDSKIKLVKNEQTLDISFSPEIEKVYRCTLIGKDAINTYMKKLSIANNHVIFRNLIKLSKKDDKIEILKKEKKSDQTIDIDDLYMRARLSSETKISDEEYKKLIAIDETQMDNIIFRYKERTSLYVYQNDKDYIRIDLTITKMDKKYNKLNNAISNYELELETMCEKPKNEILEKMYNEIEVIFKVVQQSNFIITKSEANSVIENYKNLLAVPKQNERSLYGRQPISLEIQYVESLANKYAVTDKADGERHFLVIFNNHVYLMNKNLDVKNTGIILKKEQERYNNSVIDGELIFLKNRHVFLTFDCLFNGGNDIRKEAKLEERLKNADIIINDCFVFNGQKGCKYLETEHHKEFNLNKTVEFHRKQIKMTLDNLNHDIEIEKQFLLVRRKYFIHSIGAKPWEIARYTALIWESYTGDPEIKCPYTLDGLIFQPNEQKYAVNKKDSRYDDYKWKPREKNSIDFYVEFLKDDDGNILTVYDNSYSKISDDPNAVDNAQERNANQTYRICRLHVGQIVGQLQAPVLFRENDNLYEAYIPLQNGEIRDIEGNILADKTVVEFYYNSMSNLPNKFKWIPMRTRYDKTEMVMKYKQNYGNFVSVADKVWQSIVNPVLMTDFEDLAKGNNPDKNQYFYDKKLDELRKRIGHDMIVSAAKEDKYFQLITKLGIPMRNFHNFIKSNIIFTFCHSMYQDNKQKSVLDLGCGRGGDQNRFYYAKVAFYVGVDYDRDALFNPLDSSTSRYNQQKRKPGFTKMFFIHGDQSNELDYESQFASLGGMDFQNKQLIERFFSKDPAKRTTFDVINCQFAIHYSFKNDDSLSNFKKNINNYLRNDGYVLITTFDGNAIRKLLKNKERFTQEYVNEEGEVKILFDIVKKYDDVNDDVIMGTGNAIDVHMAWLFNEGVYQTEYLVDEKFIIEEFKRDCNLELVTMDSFENQFNILKEYLTEYVQYEADDRTREGILKRAGEFYKSNSVNDGCKKYTDLEKYYVFRKNIPKAKKQKGGNNDLTNTHKYSISPLPGYDNNYSFINSIHHILKNHEIIPKTLSPKTFCSDMGIDYENDINIDSNFKKIAKNIVIYHANNDGKHEKILNGLNILLAERNKDDEYTFKIIEKGKKITQNDNVIMLAKEGVMYAPIYQIDSDLRNGIFHMDDENIKQLLELQ

SEQ ID NO: 80 - protein, 1161 aa, synthetic construct
MATKNKSDNIRNIIGLDNVSRVEEMINNFKKNRDMEFEISVRKINYSNYIRISEYFVNTTTDIQQITSLDISIILEDGNTYRISFLDEAMINDFLSKYSNMKYGDIIRYILSLNPSENIEIIYKNRGSADRLSIDDLNLVIKLTEELSLSNTTKPKLSGREKIFYRYKNRYSFTIDNTTRVDITDVKETPNIWELSRKPSNYEIELEFTNNKINSNQVFDKIFDLLKVVQNTEIPIGIRESKQVINDYQNLLNLRSSNHLDSRNVVSIESQHIIKFIPNRYAITDKADGERYFLFSTSSGIYLLSTNMTVKKVNIPVLQKDFQNMLLDGELIEIDGKELFMVFDVVYHNNIDYRYDNNYSLTHRIIVINDIIDKAFNNLIPFTDYTDKYDNLELDKIKEFYSGGIKTYWKNFNKKLKNYPGLFISRKLYFVPYGIDSSEVFMYADLVWKLCVYDQLTPYKLDGIIYTPISSPYMIKTSANELDSVPMEYKWKPPSQNSIDFFIKFDKNAKGDEAIYYDNTVVAGEGRPYKICSLYVGLNKGGEEKPIPFKVAGVEQRAFIYLTNDEALDLSGNVINDNTVVEFIFDNFKTDMDDPYKWIAVRTRYDKTESVQKYKKKYGNNLHIATRIWRTITNPVTEEIIAALGNTLTYEKEMSKLIKMHESYNKQSFAYYQKNTSNAIGMRAFNNFIKSNMIITYCKEKLSVLDIGCGRGGDLIKFIHANIREYVGLDIDNNGLYVINDSAFNRYKNLKKTNKNVPPMTFINADARGLFNVEAQEKILPNMPESNKKLINNYLSSNKRYDAINCQFTLHYYLSDDISWNNFCQNINNHIKDNGYLLITCFDGQLIYDKLKGKQKYSSSYTDNFGKKNIFFEINKIYSDEEIKPIGMPIDIYNSLISNPGTYQREYLVFPDFLQKSLKDLCGLELVETDMFYNIFNLYRNYFRINDGTFSIGEISGKRYNEIKDFYLALEGKSSSTSESDIAFASFKLAMLNRYYVFKKKTVTNLTEPSHIVSGVNKKTDLGKVLMPYFITNNMIIDHSLANNDVNKIYHIIRKKYNPVKPSVYLVRHNIIDNPMDGITFSRNKLEFIKIKNGIDPKILLIYKSPEKIFYPLYYQRFENYDSSEDYSKGDIYLKDKGTYLLDSDKIVNDLNMLVNISGKV

SEQ ID NO: 81-82 - skipped (2 entries)

SEQ ID NO: 83 - protein, 14 aa, synthetic construct
GAATAGTGAATAGT

SEQ ID NO: 84 - protein, 12 aa, synthetic construct
GAATAGTGAATA

SEQ ID NO: 85 - protein, 10 aa, synthetic construct
TAATAGTGAA

SEQ ID NO: 86 - protein, 21 aa, synthetic construct
GAATAGTGAATAGTGAATAGT

SEQ ID NO: 87 - protein, 23 aa, synthetic construct
GAATAGTPGAATAGTPGAATAGT

SEQ ID NO: 88 - protein, 5 aa, synthetic construct
GGGGS

SEQ ID NO: 89 - protein, 12 aa, synthetic construct
GGGGSSGGGGSS

SEQ ID NO: 90 - protein, 21 aa, synthetic construct
GSSAGSAALINKERATETHER

SEQ ID NO: 91 - protein, 4044 aa, synthetic construct
TCGCGCGTTTCGGTGATGACGGTGAAAACCTCTGACACATGCAGCTCCCGGAGACGGTCACAGCTTGTCTGTAAGCGGATGCCGGGAGCAGACAAGCCCGTCAGGGCGCGTCAGCGGGTGTTGGCGGGTGTCGGGGCTGGCTTAACTATGCGGCATCAGAGCAGATTGTACTGAGAGTGCACCATATGGCTAGCCCCGCGAAATGTTTAAGTTGCATTATAGGGAAATAAGAGAGAAAAGAAGAGTAAGAAGAAATATAAGACCCCGGCGCCGCCACCATGGAGGACGCCAAGAACATCAAGAAGGGCCCCGCCCCCTTCTACCCCCTGGAGGACGGCACCGCCGGCGAGCAGCTGCACAAGGCCATGAAGCGGTACGCCCTGGTGCCCGGCACCATCGCCTTCACCGACGCCCACATCGAGGTGGACATCACCTACGCCGAGTACTTCGAGATGAGCGTGCGGCTGGCCGAGGCCATGAAGCGGTACGGCCTGAACACCAACCACCGGATCGTGGTGTGCAGCGAGAACAGCCTGCAGTTCTTCATGCCCGTGCTGGGCGCCCTGTTCATCGGCGTGGCCGTGGCCCCCGCCAACGACATCTACAACGAGCGGGAGCTGCTGAACAGCATGGGCATCAGCCAGCCCACCGTGGTGTTCGTGAGCAAGAAGGGCCTGCAGAAGATCCTGAACGTGCAGAAGAAGCTGCCCATCATCCAGAAGATCATCATCATGGACAGCAAGACCGACTACCAGGGCTTCCAGAGCATGTACACCTTCGTGACCAGCCACCTGCCCCCCGGCTTCAACGAGTACGACTTCGTGCCCGAGAGCTTCGACCGGGACAAGACCATCGCCCTGATCATGAACAGCAGCGGCAGCACCGGCCTGCCCAAGGGCGTGGCCCTGCCCCACCGGACCGCCTGCGTGCGGTTCAGCCACGCCCGGGACCCCATCTTCGGCAACCAGATCATCCCCGACACCGCCATCCTGAGCGTGGTGCCCTTCCACCACGGCTTCGGCATGTTCACCACCCTGGGCTACCTGATCTGCGGCTTCCGGGTGGTGCTGATGTACCGGTTCGAGGAGGAGCTGTTCCTGCGGAGCCTGCAGGACTACAAGATCCAGAGCGCCCTGCTGGTGCCCACCCTGTTCAGCTTCTTCGCCAAGAGCACCCTGATCGACAAGTACGACCTGAGCAACCTGCACGAGATCGCCAGCGGCGGCGCCCCCCTGAGCAAGGAGGTGGGCGAGGCCGTGGCCAAGCGGTTCCACCTGCCCGGCATCCGGCAGGGCTACGGCCTGACCGAGACCACCAGCGCCATCCTGATCACCCCCGAGGGCGACGACAAGCCCGGCGCCGTGGGCAAGGTGGTGCCCTTCTTCGAGGCCAAGGTGGTGGACCTGGACACCGGCAAGACCCTGGGCGTGAACCAGCGGGGCGAGCTGTGCGTGCGGGGCCCCATGATCATGAGCGGCTACGTGAACAACCCCGAGGCCACCAACGCCCTGATCGACAAGGACGGCTGGCTGCACAGCGGCGACATCGCCTACTGGGACGAGGACGAGCACTTCTTCATCGTGGACCGGCTGAAAAGTCTGATCAAGTACAAGGGCTACCAGGTGGCCCCCGCCGAGCTGGAGAGCATCCTGCTGCAGCACCCCAACATCTTCGACGCCGGCGTGGCCGGCCTGCCCGACGACGACGCCGGCGAGCTGCCCGCCGCCGTGGTGGTGCTGGAGCACGGCAAGACCATGACCGAGAAGGAGATCGTGGACTACGTGGCCAGCCAGGTGACCACCGCCAAGAAGCTGCGGGGCGGCGTGGTGTTCGTGGACGAGGTGCCCAAGGGCCTGACCGGCAAGCTGGACGCCCGGAAGATCCGGGAGATCCTGATCAAGGCCAAGAAGGGCGGCAAGATCGCCGTGTGACTCGAGCTGGTACTGCATGCACGCAATGCTAGCTGCCCCTTTCCCGTCCTGGGTACCCCGAGTCTCCCCCGACCTCGGGTCCCAGGTATGCTCCCACCTCCACCTGCCCCACTCACCACCTCTGCTAGTTCCAGACACCTCCCAAGCACGCAGCAATGCAGCTCAAAACGCTTAGCCTAGCCACACCCCCACGGGAAACAGCAGTGATTAACCTTTAGCAATAAACGAAAGTTTAACTAAGCTATACTAACCCCAGGGTTGGTCAATTTCGTGCCAGCCACACCCTGGAGCTAGCAAAAAAAAAAAAAAAAAAAAAAAAAAAAAAAAAAAAAAAAAAAAAAAAAAAAAAAAAAAAAGAAGAGCACATGTGAGCAAAAGGCCAGCAAAAGGCCAGGAACCGTAAAAAGGCCGCGTTGCTGGCGTTTTTCCATAGGCTCCGCCCCCCTGACGAGCATCACAAAAATCGACGCTCAAGTCAGAGGTGGCGAAACCCGACAGGACTATAAAGATACCAGGCGTTTCCCCCTGGAAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGTAGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTTGAGTCCAACCCGGTAAGACACGACTTATCGCCACTGGCAGCAGCCACTGGTAACAGGATTAGCAGAGCGAGGTATGTAGGCGGTGCTACAGAGTTCTTGAAGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCCGGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTACGGGGTCTGACGCTCAGTGGAACGAAAACTCACGTTAAGGGATTTTGGTCATGAGATTATCAAAAAGGATCTTCACCTAGATCCTTTTAAATTAAAAATGAAGTTTTAAATCAAGCCCAATCTGAATAATGTTACAACCAATTAACCAATTCTGATTAGAAAAACTCATCGAGCATCAAATGAAACTGCAATTTATTCATATCAGGATTATCAATACCATATTTTTGAAAAAGCCGTTTCTGTAATGAAGGAGAAAACTCACCGAGGCAGTTCCATAGGATGGCAAGATCCTGGTATCGGTCTGCGATTCCGACTCGTCCAACATCAATACAACCTATTAATTTCCCCTCGTCAAAAATAAGGTTATCAAGTGAGAAATCACCATGAGTGACGACTGAATCCGGTGAGAATGGCAAAAGTTTATGCATTTCTTTCCAGACTTGTTCAACAGGCCAGCCATTACGCTCGTCATCAAAATCACTCGCATCAACCAAACCGTTATTCATTCGTGATTGCGCCTGAGCGAGACGAAATACGCGATCGCTGTTAAAAGGACAATTACAAACAGGAATCGAATGCAACCGGCGCAGGAACACTGCCAGCGCATCAACAATATTTTCACCTGAATCAGGATATTCTTCTAATACCTGGAATGCTGTTTTTCCGGGGATCGCAGTGGTGAGTAACCATGCATCATCAGGAGTACGGATAAAATGCTTGATGGTCGGAAGAGGCATAAATTCCGTCAGCCAGTTTAGTCTGACCATCTCATCTGTAACATCATTGGCAACGCTACCTTTGCCATGTTTCAGAAACAACTCTGGCGCATCGGGCTTCCCATACAAGCGATAGATTGTCGCACCTGATTGCCCGACATTATCGCGAGCCCATTTATACCCATATAAATCAGCATCCATGTTGGAATTTAATCGCGGCCTCGACGTTTCCCGTTGAATATGGCTCATAACACCCCTTGTATTACTGTTTATGTAAGCAGACAGTTTTATTGTTCATGATGATATATTTTTATCTTGTGCAATGTAACATCAGAGATTTTGAGACACGGGCCAGAGCTGCA

SEQ ID NO: 92 - protein, 4044 aa, synthetic construct
TCGCGCGTTTCGGTGATGACGGTGAAAACCTCTGACACATGCAGCTCCCGGAGACGGTCACAGCTTGTCTGTAAGCGGATGCCGGGAGCAGACAAGCCCGTCAGGGCGCGTCAGCGGGTGTTGGCGGGTGTCGGGGCTGGCTTAACTATGCGGCATCAGAGCAGATTGTACTGAGAGTGCACCATATGGCTAGCCCCGCGAAATTAATACGACTCACTATAGGGAAATAAGAGAGAAAAGAAGAGTAAGAAGAAATATAAGACCCCGGCGCCGCCACCATGGAGGACGCCAAGAACATCAAGAAGGGCCCCGCCCCCTTCTACCCCCTGGAGGACGGCACCGCCGGCGAGCAGCTGCACAAGGCCATGAAGCGGTACGCCCTGGTGCCCGGCACCATCGCCTTCACCGACGCCCACATCGAGGTGGACATCACCTACGCCGAGTACTTCGAGATGAGCGTGCGGCTGGCCGAGGCCATGAAGCGGTACGGCCTGAACACCAACCACCGGATCGTGGTGTGCAGCGAGAACAGCCTGCAGTTCTTCATGCCCGTGCTGGGCGCCCTGTTCATCGGCGTGGCCGTGGCCCCCGCCAACGACATCTACAACGAGCGGGAGCTGCTGAACAGCATGGGCATCAGCCAGCCCACCGTGGTGTTCGTGAGCAAGAAGGGCCTGCAGAAGATCCTGAACGTGCAGAAGAAGCTGCCCATCATCCAGAAGATCATCATCATGGACAGCAAGACCGACTACCAGGGCTTCCAGAGCATGTACACCTTCGTGACCAGCCACCTGCCCCCCGGCTTCAACGAGTACGACTTCGTGCCCGAGAGCTTCGACCGGGACAAGACCATCGCCCTGATCATGAACAGCAGCGGCAGCACCGGCCTGCCCAAGGGCGTGGCCCTGCCCCACCGGACCGCCTGCGTGCGGTTCAGCCACGCCCGGGACCCCATCTTCGGCAACCAGATCATCCCCGACACCGCCATCCTGAGCGTGGTGCCCTTCCACCACGGCTTCGGCATGTTCACCACCCTGGGCTACCTGATCTGCGGCTTCCGGGTGGTGCTGATGTACCGGTTCGAGGAGGAGCTGTTCCTGCGGAGCCTGCAGGACTACAAGATCCAGAGCGCCCTGCTGGTGCCCACCCTGTTCAGCTTCTTCGCCAAGAGCACCCTGATCGACAAGTACGACCTGAGCAACCTGCACGAGATCGCCAGCGGCGGCGCCCCCCTGAGCAAGGAGGTGGGCGAGGCCGTGGCCAAGCGGTTCCACCTGCCCGGCATCCGGCAGGGCTACGGCCTGACCGAGACCACCAGCGCCATCCTGATCACCCCCGAGGGCGACGACAAGCCCGGCGCCGTGGGCAAGGTGGTGCCCTTCTTCGAGGCCAAGGTGGTGGACCTGGACACCGGCAAGACCCTGGGCGTGAACCAGCGGGGCGAGCTGTGCGTGCGGGGCCCCATGATCATGAGCGGCTACGTGAACAACCCCGAGGCCACCAACGCCCTGATCGACAAGGACGGCTGGCTGCACAGCGGCGACATCGCCTACTGGGACGAGGACGAGCACTTCTTCATCGTGGACCGGCTGAAAAGTCTGATCAAGTACAAGGGCTACCAGGTGGCCCCCGCCGAGCTGGAGAGCATCCTGCTGCAGCACCCCAACATCTTCGACGCCGGCGTGGCCGGCCTGCCCGACGACGACGCCGGCGAGCTGCCCGCCGCCGTGGTGGTGCTGGAGCACGGCAAGACCATGACCGAGAAGGAGATCGTGGACTACGTGGCCAGCCAGGTGACCACCGCCAAGAAGCTGCGGGGCGGCGTGGTGTTCGTGGACGAGGTGCCCAAGGGCCTGACCGGCAAGCTGGACGCCCGGAAGATCCGGGAGATCCTGATCAAGGCCAAGAAGGGCGGCAAGATCGCCGTGTGACTCGAGCTGGTACTGCATGCACGCAATGCTAGCTGCCCCTTTCCCGTCCTGGGTACCCCGAGTCTCCCCCGACCTCGGGTCCCAGGTATGCTCCCACCTCCACCTGCCCCACTCACCACCTCTGCTAGTTCCAGACACCTCCCAAGCACGCAGCAATGCAGCTCAAAACGCTTAGCCTAGCCACACCCCCACGGGAAACAGCAGTGATTAACCTTTAGCAATAAACGAAAGTTTAACTAAGCTATACTAACCCCAGGGTTGGTCAATTTCGTGCCAGCCACACCCTGGAGCTAGCAAAAAAAAAAAAAAAAAAAAAAAAAAAAAAAAAAAAAAAAAAAAAAAAAAAAAAAAAAAAAGAAGAGCACATGTGAGCAAAAGGCCAGCAAAAGGCCAGGAACCGTAAAAAGGCCGCGTTGCTGGCGTTTTTCCATAGGCTCCGCCCCCCTGACGAGCATCACAAAAATCGACGCTCAAGTCAGAGGTGGCGAAACCCGACAGGACTATAAAGATACCAGGCGTTTCCCCCTGGAAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGTAGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTTGAGTCCAACCCGGTAAGACACGACTTATCGCCACTGGCAGCAGCCACTGGTAACAGGATTAGCAGAGCGAGGTATGTAGGCGGTGCTACAGAGTTCTTGAAGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCCGGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTACGGGGTCTGACGCTCAGTGGAACGAAAACTCACGTTAAGGGATTTTGGTCATGAGATTATCAAAAAGGATCTTCACCTAGATCCTTTTAAATTAAAAATGAAGTTTTAAATCAAGCCCAATCTGAATAATGTTACAACCAATTAACCAATTCTGATTAGAAAAACTCATCGAGCATCAAATGAAACTGCAATTTATTCATATCAGGATTATCAATACCATATTTTTGAAAAAGCCGTTTCTGTAATGAAGGAGAAAACTCACCGAGGCAGTTCCATAGGATGGCAAGATCCTGGTATCGGTCTGCGATTCCGACTCGTCCAACATCAATACAACCTATTAATTTCCCCTCGTCAAAAATAAGGTTATCAAGTGAGAAATCACCATGAGTGACGACTGAATCCGGTGAGAATGGCAAAAGTTTATGCATTTCTTTCCAGACTTGTTCAACAGGCCAGCCATTACGCTCGTCATCAAAATCACTCGCATCAACCAAACCGTTATTCATTCGTGATTGCGCCTGAGCGAGACGAAATACGCGATCGCTGTTAAAAGGACAATTACAAACAGGAATCGAATGCAACCGGCGCAGGAACACTGCCAGCGCATCAACAATATTTTCACCTGAATCAGGATATTCTTCTAATACCTGGAATGCTGTTTTTCCGGGGATCGCAGTGGTGAGTAACCATGCATCATCAGGAGTACGGATAAAATGCTTGATGGTCGGAAGAGGCATAAATTCCGTCAGCCAGTTTAGTCTGACCATCTCATCTGTAACATCATTGGCAACGCTACCTTTGCCATGTTTCAGAAACAACTCTGGCGCATCGGGCTTCCCATACAAGCGATAGATTGTCGCACCTGATTGCCCGACATTATCGCGAGCCCATTTATACCCATATAAATCAGCATCCATGTTGGAATTTAATCGCGGCCTCGACGTTTCCCGTTGAATATGGCTCATAACACCCCTTGTATTACTGTTTATGTAAGCAGACAGTTTTATTGTTCATGATGATATATTTTTATCTTGTGCAATGTAACATCAGAGATTTTGAGACACGGGCCAGAGCTGCA

(19) United States
(12) Patent Application Publication
(10) Pub. No.: US 2025/0305019 A1
(43) Pub. Date: Oct. 2, 2025

(54) COMPOSITIONS, KITS, AND METHODS FOR IN VITRO TRANSCRIPTION

(71) Applicant: New England Biolabs, Inc., Ipswich, MA (US)

(72) Inventors: Dillon B. Nye, Somerville, MA (US); Jennifer L. Curcuru, Gloucester, MA (US); Tien-Hao Chen, Wakefield, MA (US); Lili Mitchell, Ipswich, MA (US); Ivan R. Correa, JR., Hamilton, MA (US)

(73) Assignee: New England Biolabs, Inc., Ipswich, MA (US)

(21) Appl. No.: 19/095,861

(22) Filed: Mar. 31, 2025

Related U.S. Application Data
(60) Provisional application No. 63/571,752, filed on Mar. 29, 2024.

(51) Int. Cl. C12P 21/00; C12N 9/10; C12N 9/12; C12N 15/52

(52) U.S. Cl. CPC C12P 21/00; C12N 9/1007; C12N 9/1247; C12N 15/52; C12Y 207/07006

(57) ABSTRACT

The present disclosure relates, according to some embodiments, to cold-active RNA polymerases, variants thereof, compositions and kits comprising cold-active RNA polymerases, and methods of using cold-active RNA polymerases. Cold-active polymerases may have, for example, an amino acid sequence ≥90%, ≥91%, ≥92%, ≥93%, ≥94%, ≥95%, ≥96%, ≥97%, ≥98%, ≥99% identical (e.g., ≥95% or ≥98% identical) to any of SEQ ID NOS: 1-19 and optionally may have at least one substitution relative to SEQ ID NO:1.

### CROSS-REFERENCE TO RELATED APPLICATIONS

This application also claims priority to U.S. Provisional Application No. 63/571,752 filed Mar. 29, 2024, the entire contents of which are hereby incorporated in their entirety by reference.

### SEQUENCE LISTING STATEMENT

This disclosure includes a Sequence Listing submitted electronically in .xml format under the file name “NEB-487-US.xml” created on Mar. 31, 2025, and having a size of 219,978 bytes. This Sequence Listing is incorporated herein in its entirety by this reference.

### BACKGROUND

mRNA therapeutics can be tailored to induce cellular expression of any encoded protein, representing a customizable and adaptable drug modality that is rapidly maturing. Therapeutic mRNAs have applications as cancer immunotherapies, infectious disease vaccines, protein replacement treatments and gene editing tools. The synthetic mRNA drug substance is required to mimic eukaryotic transcripts and typically contains an N7-methylguanosine cap, 5′ and 3′ untranslated regions, and a 3′ polyadenosine tail. These structural elements are desirable for reducing or preventing RNA degradation and/or promoting RNA translation. Typically, the mRNA is synthesized by in vitro transcription (IVT) using T7 RNA polymerase or one of its variants. The designed mRNA sequence is encoded on a linearized plasmid which contains the T7 promoter and is used as an IVT template to produce large amounts of RNA. Covalent modifications at the 5′ terminus can be installed co-transcriptionally by supplying the IVT reaction with cap analogs, or added in subsequent enzymatic reactions, or eschewed altogether by circularizing the RNA and including an internal ribosome entry site. The mRNA can be designed to include the replication machinery and subgenomic promoter of an alphavirus, yielding a large self-amplifying RNA (saRNA) molecule. Modified nucleotides such as pseudouridine (ψ) can be incorporated into the mRNA to attenuate unwanted immunostimulatory properties of the drug substance. Whatever forms the mRNA therapy takes, its design and production are beholden to the capabilities of T7 RNA polymerase.

### SUMMARY

Accordingly, needs have arisen for improved RNA polymerases, for example, cold-active RNA polymerases and/or RNA polymerases that produce transcripts with desirable properties (e.g., transcripts having reduced immunogenicity when administered to humans or other mammals). The present disclosure relates, in some embodiments, to cold-active RNA polymerases and variants thereof. For example, cold-active polymerases may have an amino acid sequence ≥90%, ≥91%, ≥92%, ≥93%, ≥94%, ≥95%, ≥96%, ≥97%, ≥98%, ≥99% identical (e.g., ≥95% or ≥98% identical) to any of SEQ ID NOS: 1-29 (e.g., any of SEQ ID NOS: 1-19; any of SEQ ID NOS: 1-15) and may have at least one substitution relative to SEQ ID NO:1. In some embodiments, a variant cold-active RNA polymerase may have (a) an amino acid sequence ≥90%, ≥91%, ≥92%, ≥93%, ≥94%, ≥95%, ≥96%, ≥97%, ≥98%, ≥99% identical (e.g., ≥95% or ≥98% identical) to any of SEQ ID NOS: 1-29 (e.g., any of SEQ ID NOS: 1-19; any of SEQ ID NOS: 1-15), and (b) optionally at least one conservative substitution relative to SEQ ID NO: 1. A variant cold-active RNA polymerase may have (a) an amino acid sequence ≥90%, ≥91%, ≥92%, ≥93%, ≥94%, ≥95%, ≥96%, ≥97%, ≥98%, ≥99% identical (e.g., ≥95% or ≥98% identical) to any of SEQ ID NOS: 1-29 (e.g., any of SEQ ID NOS: 1-19; any of SEQ ID NOS: 1-15), and (b) at least one conservative substitution relative to SEQ ID NO:1 or at least two conservative substitutions relative to SEQ ID NO:1. Optionally, a variant cold-active RNA polymerase may have a third substitution relative to SEQ ID NO:1. In some embodiments, a variant cold-active RNA polymerase may be fused to another polypeptide or protein. According to some embodiments, a fusion may comprise, in an N-terminal to C-terminal direction, (I) a purification tag or a sorting signal peptide, and (II) any of the cold-active RNA polymerases set forth in this paragraph (or otherwise disclosed herein) operably linked to (I) or a fusion may comprise, in an N-terminal to C-terminal direction, (III) any of the cold-active RNA polymerases set forth in this paragraph (or otherwise disclosed herein) and (IV) a purification tag or a sorting signal peptide operably linked to (III).

The present disclosure also relates, in some embodiments, to compositions comprising any of the cold-active RNA polymerases, variants thereof or fusions thereof set forth in the preceding paragraph (or otherwise disclosed herein). For example, a composition may comprise a cold-active RNA polymerase (or a variant thereof or a fusion thereof) and a template comprising a sequence encoding an RNA of interest. A template may comprise (e.g., may further comprise) a cold-active RNA polymerase promoter. Example cold-active RNA polymerase promoters include promoters having any of the nucleotide sequences of SEQ ID NOS: 31-46. Compositions, according to some embodiments, may comprise at least one of a buffering agent and a polyamine or may comprise both a buffering agent and a polyamine. Example buffering agents include HEPES, MES, MOPS, TAPS, tricine, Tris, ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, and triethanolamine. Example polyamines include spermidine, spermine, putrescine, polyethylenimine, 1,4,7-triazacyclononane, cyclen, ethylenediamine, and 1, 3, 5,-triazinane.

The present disclosure further relates to methods of making an RNA of interest. Methods may comprise, according to some embodiments, contacting a cold-active RNA polymerase having an amino acid sequence ≥90%, ≥91%, ≥92%, ≥93%, ≥94%, ≥95%, ≥96%, ≥97%, ≥98%, ≥99% identical (e.g., ≥95% or ≥98% identical) to any of SEQ ID NOS: 1-29; a template comprising a nucleotide (e.g., DNA) sequence encoding the RNA of interest; optionally, one or more NTPs; optionally, one or more modified NTPs; and optionally, a buffer, to produce the RNA of interest, wherein the contacting is at a temperature in a range of 18° C.-32° C. and/or for a time in the range of seconds to hours and/or the RNA of interest is a therapeutic RNA. A template, in some embodiments, may comprise (e.g., may further comprise) a cold-active RNA polymerase promoter. Example cold-active RNA polymerase promoters include promoters having any of the nucleotide sequences of SEQ ID NOS: 31-46. In some embodiments, an RNA of interest may be capped (e.g., comprise a capped RNA). For example, contacting may further comprise contacting the polymerase, the template, the optional components, if present, and a capping enzyme to produce the capped RNA. A method may comprise, in some embodiments, contacting the RNA of interest with one or more pharmaceutically acceptable additives.

The present disclosure also relates to cold-active RNA polymerase kits, according to some embodiments. A kit may comprise, for example, any cold-active RNA polymerase disclosed herein (optionally in a storage buffer) and one or more NTPs. A kit may comprise one or more modified NTPs (e.g., wTP) and/or a capping enzye (e.g., VCE or FCE). A kit, in some embodiments, may comprise a reaction buffer (e.g., a reaction buffer comprising a buffering agent and a polyamine). Example buffering agents include HEPES, MES, MOPS, TAPS, tricine, Tris, ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, and triethanolamine. Example polyamines include spermidine, spermine, putrescine, polyethylenimine, 1,4,7-triazacyclononane, cyclen, ethylenediamine, and 1, 3, 5,-triazinane.

The present disclosure, in some embodiments, relates to compositions comprising a cold-active RNA polymerase (a cold-active RNA polymerase having an amino acid sequence ≥90, ≥95, ≥98% identical to any of SEQ ID NOS: 1-19) and a capping enzyme. A cold-active RNA polymerase may be a non-naturally occurring cold-active RNA polymerase or a cold-active RNA polymerase of Pseudomonas phage Njord, Pseudomonas phage Nerthus, Pseudomonas phage Alpheus, Pseudomonas phage Achelous, Pseudomonas phage uligo, Vibrio phage qA318, Vibrio phage qAS51, Vibrio phage Vp670, Vibrio phage Vc1, Vibrio phage VEN, Marinomonas phage CPP1m, Marinomonas phage CB5A, Pseudomonas phage Ulina01, Pseudomonas phage Ulitu01, or Pseudomonas phage BUCT553. A capping enzyme may be a non-naturally occurring capping enzyme or a single chain capping enzyme (e.g., a capping enzyme of Faustovirus, mimivirus, or moumouvirus). In some embodiments, a cold-active RNA polymerase may have an amino acid sequence that is (a) at least 95% identical to any of SEQ ID NOS: 1-19, (b) 100% identical to SEQ ID NO:17, and/or (C)<100% identical to each of SEQ ID NOS: 1-15. A cold-active RNA polymerase may have, according to some embodiments, ≥1, ≥2, ≥3, ≥4, ≥5, ≥6, ≥8, ≥10, ≥12, ≥15, ≥18, ≥20 conservative substitutions relative to SEQ ID NO:1. A capping enzyme may have an amino acid sequence ≥90, ≥95, ≥98% identical to any of SEQ ID NOS: 75-80, according to some embodiments. In some embodiments, a cold-active RNA polymerase may be immobilized to a support or a capping enzyme may be immobilized to a support or the polymerase and the capping enzyme each may be immobilized to a separate support or the polymerase and the capping enzyme each may be immobilized to a common support (e.g., with each attached separately to the support or with the polymerase and the capping enzyme fused and the fusion attached to the support). A composition, in some embodiments, may comprise a fusion protein, wherein the fusion protein comprises the cold-active RNA polymerase and the capping enzyme (e.g., in an N-terminal to C-terminal direction, (a) the polymerase and the capping enzyme or (b) the capping enzyme and the polymerase), optionally with or without a linker disposed between the polymerase and the capping enzyme. A composition may comprise one or more components suitable for capping reactions including, for example, guanosine triphosphate (GTP) or modified GTP, a methyl group donor (e.g., S-adenosyl methionine), a 2′ O-methyltransferase, and a buffering agent. A composition may comprise one or more components suitable for a transcription reaction including, for example, a polynucleotide template (e.g., comprising, in a 5′ to 3′ direction, a promoter corresponding to the polymerase and a sequence of interest), NTPs, a cap analog, and a buffering agent. A promoter may have a nucleotide sequence according to one of SEQ ID NOS: 31-46 (or a sequence ≥85%, ≥90%, or ≥95% identical thereto) and/or wherein the sequence of interest comprises a coding sequence. Example coding sequences include therapeutic protein coding sequences, vaccine protein coding sequence (e.g., proteins that trigger a desirable immune response, for example, conferring resistance to a microbial infection), replacement protein coding sequence (e.g., a protein or enzyme that is defective or missing in a host cell of interest), supplemental protein coding sequences (e.g., a protein or enzyme that is present in a host cell of interest, but in insufficient quantites). In some embodiments, a composition may comprise a polyribonucleotide product of the polymerase (e.g., a transcription product). A polyribonucleotide product of the polymerase, in some embodiments, may have fewer double-stranded RNA molecules (e.g., fewer polyribonucleotide product molecules comprising a double-stranded region, fewer double-stranded regions per molecule, and/or shorter double-stranded regions) than a polyribonucleotide product of T7 RNA polymerase having the same nucleotide sequence. In some embodiments, a composition may comprise at least one of a buffering agent and/or a polyamine. Examples buffering agents include or comprise HEPES, MES, MOPS, TAPS, tricine, Tris, ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, or triethanolamine. Examples polyamines include or comprise spermidine, spermine, putrescine, polyethylenimine, 1,4,7-triazacyclononane, cyclen, ethylenediamine, or 1, 3, 5,-triazinane.

The present disclosure relates, in some embodiments, to fusions comprising a polymerase (e.g., a naturally or non-naturally occurring cold-active RNA polymerase) and a capping enzyme. A fusion may comprise, for example, an N-terminal polymerase and a C-terminal capping enzyme or an N-terminal capping enzyme and a C-terminal polymerase.

In some embodiments, a fusion may comprise (a) in an N-terminal to C-terminal direction, (i) a polymerase, wherein the polymerase (1) is a non-naturally occurring cold-active RNA polymerase or a cold-active RNA polymerase of Pseudomonas phage Njord, Pseudomonas phage Nerthus, Pseudomonas phage Alpheus, Pseudomonas phage Achelous, Pseudomonas phage uligo, Vibrio phage qA318, Vibrio phage AS51, Vibrio phage Vp670, Vibrio phage Vc1, Vibrio phage VEN, Marinomonas phage CPPIm, Marinomonas phage CB5A, Pseudomonas phage Ulina01, Pseudomonas phage Ulitu01, or Pseudomonas phage BUCT553; and (2) has an amino acid sequence at least 90%, at least 95%, or at least 98% identical to any of SEQ ID NOS: 1-19; and (ii) a capping enzyme, wherein the capping enzyme is a non-naturally occurring capping enzyme or a capping enzyme of Faustovirus, mimivirus, or moumouvirus (e.g., having n amino acid sequence at least 90%, at least 95%, or at least 98% identical to any of SEQ ID NOS: 75-78); or (b) in an N-terminal to C-terminal direction, the capping enzyme and the polymerase. For example, a fusion may comprise (e.g., in an N-terminal to C-terminal direction) a cold active RNA polymerase and a capping enzyme, wherein the polymerase has an amino acid sequence at least 98% identity to SEQ ID NO:1 and/or the capping enzyme has at least 98% identity to SEQ ID NO:75. In some embodiments, a fusion may be immobilized to a support (e.g., a magnetic bead, a surface of a container) with or without a linker (e.g., a linker disposed between the fusion and the support). A fusion may include, according to some embodiments, a purification tag or a sorting signal peptide. For example, a fusion may comprise in an N-terminal to C-terminal direction, the purification tag or the sorting signal, the polymerase, and the capping enzyme, or in an N-terminal to C-terminal direction, the purification tag or the sorting signal, the capping enzyme and the polymerase.

The present disclosure further provides kits including a cold-active RNA polymerase and one or more other materials (e.g., materials for a cold-active RNA polymerase reaction). A kit may include, for example, any of the disclosed cold-active RNA polymerases including disclosed fusions and (optionally) one or more of guanosine triphosphate (GTP) or modified GTP; a methyl group donor; a 2′ O-methyltransferase; and a buffering agent. A kit may include a support to which a cold-active RNA polymerase and/or a cold-active RNA polymerase fusion may be immobilized or a kit may include an immobilized cold-active RNA polymerase and/or a cold-active RNA polymerase fusion.

The present disclosure further provides methods of using a cold-active RNA polymerase and/or a cold-active RNA polymerase fusion. A method may include, for example, contacting an RNA polymerase (e.g., a cold-active RNA polymerase having an amino acid sequence at least 90%, at least 95%, or at least 98% identical to any of SEQ ID NOS: 1-19) and a polynucleotide template comprising an expression control sequence of the RNA polymerase (e.g., operable, together with the polymerase, to initiate transcription) and a coding sequence operably linked to the expression control sequence to produce a transcript (e.g., an elicitor transcript that is heterologous to and/or translatable by a mammalian cell and/or operable to elicit desired reaction or have a desired effect on such cell). A method may further comprise contacting the transcript (e.g., elicitor transcript) with the mammalian cell to form a translation product of the elicitor transcript. In some embodiments, a mammalian cell contacted with a transcript (e.g., elicitor transcript) that arises from a cold-active RNA polymerase and/or a cold-active RNA polymerase fusion may produce smaller quantities of at least one cytokine than a reference mammalian cell (e.g., a reference mammalian cell comprising a reference transcript, wherein the reference transcript is produced by contacting the same polynucleotide template under the same conditions except with T7 RNA polymerase instead of the cold-active RNA polymerase). In some embodiments, the transcript (e.g., elicitor transcript) may be produced in vitro by cold-active RNA polymerase and/or a cold-active RNA polymerase fusion at a first temperature (e.g., 25° C.) and the reference transcript may be produced by T7 RNA polymerase at a second temperature (e.g., 37° C.). In some embodiments, a translation product of a transcript (e.g., an elicitor transcript) expressible in a mammalian cell may have a desired (e.g., therapeutic, cytotoxic) effect on the cell in which is is formed. A method may include contacting the transcript (e.g., elicitor transcript) with a mammalian cell that is contiguous with and/or in communication (e.g., in fluid communication) with other mammalian cells in a mammal. For example, a method may include contacting the transcript (e.g., elicitor transcript) in situ with a mammalian cell of the respiratory system, circulatory system, immune system, digestive system, nervous system, integumentary system, musculoskeletal system, excretory system, cardiovascular system, heart, the nervous system, and/or the endocrine system. In some embodiments, a coding sequence may be operable to give rise to and/or a transcript (e.g., elicitor transcript) may comprise a therapeutic RNA and/or a vaccine RNA. In some embodiments, a coding sequence may be operable to give rise to and/or a transcript (e.g., elicitor transcript) may be an artificial transcript.

A method may include, according to some embodiments, contacting (i) an RNA polymerase at least 90%, at least 95%, or at least 98% identical to any of SEQ ID NOS: 1-19; (ii) a polynucleotide template comprising an expression control sequence of the RNA polymerase and a coding sequence encoding an artificial transcript, the coding sequence operably linked to the expression control sequence; and (iii) ribonucleotide triphosphates, to produce the artificial transcript. A method may further comprise (b) contacting the artificial transcript with a capping enzyme and one or more of (i) guanosine triphosphate (GTP) or modified GTP, (ii) a methyl group donor, (iii) a 2′ O-methyltransferase, and (iv) a buffering agent, to produce a capped artificial transcript.

### BRIEF DESCRIPTION OF THE FIGURES

FIG. 1A shows an example phylogenetic tree of Molineuxvirinae and Colwellvirinae bacteriophages. Phage T7 was included and used to root the tree. The scale bar of 0.25 represents the phylogenomic distance (Bioinformatics 33, 3396-3404 (2017)).

FIG. 1B shows example results of an assessment of the locations of transcriptional promoters in the genomes of phages T7, SP6 and Njord. Open reading frames are denoted with blocks and promoters are indicated with arrows. Representative promoter sequences are shown with the initiating guanosine underlined (T7 SEQ ID NO:69; SP6 SEQ ID NO: 70; Njord SEQ ID NO:38). “CA-RNAP” refers to cold-active RNA polymerase. FIG. 1C shows an example SDS-PAGE analysis of T7 RNAP and cold-active RNA polymerase preparations. FIG. 1D shows results of example IVT reactions performed with T7 and cold-active RNA polymerases at various temperatures but otherwise identical conditions. IVT reactions were performed in triplicate and RNA yield was measured using a dye-based assay. IVT was performed using a Firefly luciferase template (T7 SEQ ID NO:48; CA-RNAP SEQ ID NO:47) to produce RNA SEQ ID NO:49, or using a self-amplifying RNA template (T7 SEQ ID NO:57; CA-RNAP SEQ ID NO:56) to produce RNA SEQ ID NO: 58. FIG. 1E shows an example agarose gel electrophoresis analysis of transcripts produced using cold-active RNA polymerase with unmodified or modified nucleotides. IVT was performed as indicated for FIG. 1D.

FIG. 2 shows example results of experiments examining the RNA yield from IVT reactions performed with variations of a Njord promoter. The sequence of the reference Njord promoter (SEQ ID NO:33) appears on the bottom with positions numbered according to the T7 promoter convention. IVT was performed using a promoter optimization template (SEQ ID NO:51) to produce RNA SEQ ID NO:52. Each bar represents the RNA yield when the reference nucleotide at that position is replaced by the nucleotide indicated in color. Yields are presented relative to the yield of the reference promoter, indicated by a dashed line. Error bars represent the standard error of three technical replicates.

FIG. 3A and FIG. 3B show results of example error profiles in nucleotide incorporation of IVT reactions with uridine analogs. Cold-active RNA polymerase and T7 RNA polymerase incorporate uridine analogs with varying error rates during in vitro transcription. RNA transcripts produced by each enzyme were converted into cDNA with ProtoScript II reverse transcriptase and the cDNAs produced were subjected to library preparation and Pacific Biosciences SMRT sequencing. FIG. 3A shows total combined error rates with uridine and the indicated analogs. FIG. 3B shows a base substitution profile for each polymerase with specific substitutions as indicated in the legend. Results reflect 4 independent experiments, including two IVT reactions using template Fidelity 1 (T7 SEQ ID NO:54; CA-RNAP SEQ ID NO:53) to produce RNA SEQ ID NO:55 and two IVT reactions using template Fidelity 2 (T7 SEQ ID NO:57; CA-RNAP SEQ ID NO:56) to produce RNA SEQ ID NO:58. “CA” refers to cold-active RNA polymerase.

FIG. 4A, FIG. 4B, and FIG. 4C show aspects of cap analog incorporation by T7 and cold-active RNA polymerases. FIG. 4A shows structures of cap analogs and template sequences (SEQ ID NOS: 43-46 and 71-74) used for an example demonstration of co-transcriptional capping. “CA-RNAP” refers to cold-active RNA polymerase. FIG. 4B shows the mass of RNA (μg) produced by in vitro transcription reactions with T7 (left) and cold-active RNA polymerase (right) RNA polymerases using the indicated templates in the presence or absence of trinucleotide cap analogs. Below, the proportions of 5′ capped mRNA observed in reactions including the trinucleotide cap analogs is shown as determined by mass spectrometry. The trinucleotide cap analogs and the template sequences used are indicated by the dinucleotide sequence. FIG. 4C shows the proportions of 5′ capped mRNA observed in reactions with varying concentrations of ARCA using a self-amplifying RNA template (T7 SEQ ID NO:57; CA-RNAP SEQ ID NO:56) to produce RNA SEQ ID NO: 58.

FIG. 5A and FIG. 5B show example production of double-stranded RNA by T7 and cold-active RNA polymerases. FIG. 5A shows native polyacrylamide gel electrophoresis analysis of unstructured RNA (SEQ ID NO:67) produced by T7 and cold-active RNA polymerases using a template (T7 SEQ ID NO:65; CA-RNAP SEQ ID NO:66). Acridine orange was used to stain the gel and fluorescence emission was collected at two wavelengths to visualize single-stranded RNA (670 nm) and double-stranded RNA (525 nm). Digestion of the reactions by either RNase If or RNase III confirmed production of dsRNA by T7 RNAP and not cold-active RNA polymerase. “CA-RNAP” refers to cold-active RNA polymerase. FIG. 5B shows homogeneous time-resolved fluorescence measurement of double-stranded RNA reactions produced by T7 and cold-active RNA polymerases. The ratio of fluorescence emission at 665 nm and 620 nm is proportional to the amount of double-stranded RNA present in the IVT reaction. IVT reactions were performed using C. luciferase templates (T7 SEQ ID NO:63; CA-RNAP SEQ ID NO:62) to produce RNA SEQ ID NO:64 and self-amplifying RNA templates (T7 SEQ ID NO:60; CA-RNAP SEQ ID NO:59) to produce RNA SEQ ID NO:61. Abbreviations: IVT−CTC=IVT with co-transcriptional capping; IVT−PTEC=IVT with post-transcriptional enzymatic capping; and CA−RNAP=Cold active RNA polymerase.

FIG. 6A and FIG. 6B shows the effect of transfection of example luciferase mRNA preparations into A549 human lung cancer cell culture. The mRNA species (SEQ ID NO: 50) were prepared by reaction of appropriate templates (T7 SEQ ID NO:92; CA-RNAP SEQ ID NO:91) with either T7 RNAP or cold-active RNAP polymerase in the presence of either unmodified uridine (U) or N1-methylpseudouridine (N1 ml). “CA-RNAP” refers to cold-active RNA polymerase. FIG. 6A shows expression of the delivered mRNA measured by detecting activity in cell lysates of the firefly luciferase enzyme encoded by the mRNA. The expression levels are reported relative to a control Renilla luciferase mRNA which was co-transfected with the test mRNA. Regardless of polymerase used or nucleotide modification, the expression levels are not significantly different. Data point icons represent independent mRNA preparations. FIG. 6B shows secreted cytokine interferon beta (IFN-B) measured in the culture supernatant using an enzyme-linked immunosorbent assay. Data points lying on the y-axis represent levels below the detection limit of the assay.

FIG. 7A and FIG. 7B illustrate the results from complexing firefly luciferase mRNA with a commercial lipid reagent and introducing the mRNA nanoparticles into mice. The mRNA species (SEQ ID NO:50) were prepared by reaction of appropriate templates (T7 SEQ ID NO:92; CA-RNAP SEQ ID NO:91) with either T7 RNAP or cold-active RNAP polymerase in the presence of either unmodified uridine (U) or N1-methylpseudouridine (N1 ml). “CA-RNAP” refers to cold-active RNA polymerase and “Vehicle” refers to the lipid reagent without mRNA. Mice that received a lower dose of the mRNA nanoparticles were also injected with luciferin. In vivo images revealed expression in the liver. FIG. 7A shows the luciferase expression levels obtained by treating the mice with luciferin and assessing signal intensity of collected images. In parallel, interferon alpha (IFN-A) concentration was measured in serum collected from mice that received a higher dose of the mRNA nanoparticles. FIG. 7B shows the results of quantifying IFN-A concentration with an enzyme-linked immunosorbent assay.

FIG. 8 shows results of an example co-transcriptional capping reaction in which either a cold-active RNA polymerase plus Faustovirus capping enzyme (FCE) or a cold-active RNA polymerase::FCE fusion was used to prepare capped RNA. Reaction products were treated with RNase 4 and loaded on a denaturing polyacrylamide gel for short RNA species. Lane 1 was loaded with a microRNA ladder (sizes 17, 21, and 25 nt). Lane 2 is the 5′ fragment of an RNA species synthesized by a cold-active RNA polymerase alone; the prominent band represents the results uncapped transcript. Lane 3 is the 5′ fragment of an RNA species synthesized by a cold-active RNA polymerase and subsequently reacted with a Faustovirus capping enzyme; the prominent band represents 5′ N7-methylguanosine capped transcript. Lane 4 is the 5′ fragment of an RNA species synthesized and co-transcriptionally capped by a cold-active RNA polymerase::FCE fusion. Bands corresponding to the uncapped product of lane 2 and the capped product of lane 3 demonstrate the fusion has capping activity, although under the specific conditions tested did not cap 100% of the available RNA.

### BRIEF DESCRIPTION OF THE SEQUENCES

Some embodiments of this disclosure relate to the following provided sequences of example polynucleotides and/or example polypeptides.

SEQ ID NO:1 is an example cold-active RNA polymerase of Pseudomonas phage Njord.

SEQ ID NO:2 is an example cold-active RNA polymerase of Pseudomonas phage Nerthus.

SEQ ID NO:3 is an example cold-active RNA polymerase of Pseudomonas phage Alpheus.

SEQ ID NO:4 is an example cold-active RNA polymerase of Pseudomonas phage Achelous.

SEQ ID NO:5 is an example cold-active RNA polymerase of Pseudomonas phage uligo.

SEQ ID NO:6 is an example cold-active RNA polymerase of Vibrio phage qA318.

SEQ ID NO:7 is an example cold-active RNA polymerase of Vibrio phage PAS51.

SEQ ID NO:8 is an example cold-active RNA polymerase of Vibrio phage Vp670.

SEQ ID NO:9 is an example cold-active RNA polymerase of Vibrio phage Vc1.

SEQ ID NO:10 is an example cold-active RNA polymerase of Vibrio phage VEN.

SEQ ID NO:11 is an example cold-active RNA polymerase of Marinomonas phage CPP1m.

SEQ ID NO:12 is an example cold-active RNA polymerase of Marinomonas phage CB5A.

SEQ ID NO:13 is an example cold-active RNA polymerase of Pseudomonas phage Ulina01.

SEQ ID NO:14 is an example cold-active RNA polymerase of Pseudomonas phage Ulitu01.

SEQ ID NO:15 is an example cold-active RNA polymerase of Pseudomonas phage BUCT553.

SEQ ID NO:16 is an example variant cold-active RNA polymerase with a his tag.

SEQ ID NO:17 is an example variant cold-active RNA polymerase consensus sequence, wherein B=D or N, J=I or L, Z=Q or E, X=any residue.

SEQ ID NO:18 is an example variant cold-active RNA polymerase consensus sequence.

SEQ ID NO:19 is an example variant cold-active RNA polymerase consensus sequence.

SEQ ID NO:20 is an example cold-active RNA polymerase and Faustovirus capping enzyme fusion.

SEQ ID NO:21 is an example fusion protein comprising a cold-active RNA polymerase, a linker, and a capping enzyme.

SEQ ID NO:22 is an example fusion protein comprising a cold-active RNA polymerase, a linker, and a capping enzyme.

SEQ ID NO:23 is an example fusion protein comprising a cold-active RNA polymerase, a linker, and a capping enzyme.

SEQ ID NO:24 is an example fusion protein comprising a cold-active RNA polymerase, a linker, and a capping enzyme.

SEQ ID NO:25 is an example fusion protein comprising a cold-active RNA polymerase, a linker, and a capping enzyme.

SEQ ID NO:26 is an example fusion protein comprising a cold-active RNA polymerase, a linker, and a capping enzyme.

SEQ ID NO:27 is an example fusion protein comprising a cold-active RNA polymerase, a linker, and a capping enzyme.

SEQ ID NO:28 is an example fusion protein comprising a cold-active RNA polymerase, a linker, and a capping enzyme.

SEQ ID NO:29 is an example fusion protein comprising a cold-active RNA polymerase, a linker, and a capping enzyme.

SEQ ID NO:30 is an example cold-active RNA polymerase expression plasmid.

SEQ ID NO:31 is an example cold-active RNA polymerase promoter sequence (1).

SEQ ID NO:32 is an example cold-active RNA polymerase promoter sequence (2).

SEQ ID NO:33 is an example cold-active RNA polymerase promoter sequence (3).

SEQ ID NO:34 is an example cold-active RNA polymerase promoter sequence (4).

SEQ ID NO:35 is an example cold-active RNA polymerase promoter sequence (5).

SEQ ID NO:36 is an example cold-active RNA polymerase promoter sequence (6).

SEQ ID NO:37 is an example cold-active RNA polymerase promoter sequence (7).

SEQ ID NO:38 is an example cold-active RNA polymerase promoter sequence (8).

SEQ ID NO:39 is an example cold-active RNA polymerase promoter sequence (Core 1).

SEQ ID NO:40 is an example cold-active RNA polymerase promoter sequence (Core 2).

SEQ ID NO:41 is an example cold-active RNA polymerase promoter sequence (Core 3).

SEQ ID NO:42 is an example cold-active RNA polymerase promoter sequence.

SEQ ID NO:43 is an example cold-active RNA polymerase template promoter sequence (GG).

SEQ ID NO:44 is an example cold-active RNA polymerase template promoter sequence (GU).

SEQ ID NO:45 is an example cold-active RNA polymerase template promoter sequence (AG).

SEQ ID NO:46 is an example cold-active RNA polymerase template promoter sequence (AU).

SEQ ID NO:47 is an example cold-active RNA polymerase transcription template (Firefly luciferase).

SEQ ID NO:48 is an example T7 RNA polymerase transcription template (Firefly luciferase).

SEQ ID NO:49 is an example transcription product of Firefly luciferase SEQ ID NO:50 is an example firefly luciferase suitable for transfection of mammalian cells.

SEQ ID NO:51 is an example cold-active RNA polymerase transcription template (Promoter optimization template).

SEQ ID NO:52 is an example transcription product of promoter optimization template

SEQ ID NO:53 is an example cold-active RNA polymerase transcription template (Fidelity 1).

SEQ ID NO:54 is an example T7 RNA polymerase transcription template (Fidelity 1).

SEQ ID NO:55 is an example transcription product of Fidelity 1

SEQ ID NO:56 is an example cold-active RNA polymerase transcription template (Fidelity 2).

SEQ ID NO:57 is an example T7 RNA polymerase transcription template (Fidelity 2).

SEQ ID NO:58 is an example transcription product of Fidelity 2

SEQ ID NO:59 is an example cold active RNA polymerase transcription template, self-amplifying RNA.

SEQ ID NO:60 is an example T7 transcription template, self-amplifying RNA.

SEQ ID NO:61 is an example cold-active RNA polymerase and T7 transcription product, self-amplifying RNA.

SEQ ID NO:62 is an example cold-active RNA polymerase transcription template (C. luciferase).

SEQ ID NO:63 is an example T7 RNA polymerase transcription template (C. luciferase).

SEQ ID NO:64 is an example transcription product of C. luciferase.

SEQ ID NO:65 is an example T7 RNA polymerase transcription template (unstructured)

SEQ ID NO:66 is an example cold-active RNA polymerase transcription template (unstructured)

SEQ ID NO:67 is an example transcription product of unstructured RNA SEQ ID NO:68 is an example oligonucleotide used for measurement of 5′ capping efficiency.

SEQ ID NO:69 is an example T7 RNA polymerase promoter sequence (initiating guanosine underlined).

SEQ ID NO:70 is an example SP6 RNA polymerase promoter sequence (initiating guanosine underlined).

SEQ ID NO:71 is an example T7 RNA polymerase template promoter sequence (GG).

SEQ ID NO:72 is an example T7 RNA polymerase template promoter sequence (GU).

SEQ ID NO:73 is an example T7 RNA polymerase template promoter sequence (AG).

SEQ ID NO:74 is an example T7 RNA polymerase template promoter sequence (AU).

SEQ ID NO:75 is an example Faustovirus D5b capping enzyme.

SEQ ID NO:76 is an example Faustovirus E12 capping enzyme.

SEQ ID NO:77 is an example Faustovirus ST1 capping enzyme.

SEQ ID NO:78 is an example Faustovirus LC9 capping enzyme.

SEQ ID NO:79 is an example mimivirus capping enzyme.

SEQ ID NO:80 is an example moumouvirus capping enzyme.

SEQ ID NO:81 is an example linker usable in fusion proteins (GAATAGT).

SEQ ID NO:82 is an example linker usable in fusion proteins (GAATAG).

SEQ ID NO:83 is an example linker usable in fusion proteins.

SEQ ID NO:84 is an example linker usable in fusion proteins.

SEQ ID NO:85 is an example linker usable in fusion proteins.

SEQ ID NO:86 is an example linker usable in fusion proteins.

SEQ ID NO:87 is an example linker usable in fusion proteins.

SEQ ID NO:88 is an example linker usable in fusion proteins.

SEQ ID NO:89 is an example linker usable in fusion proteins.

SEQ ID NO:90 is an example linker usable in fusion proteins.

SEQ ID NO:91 is an example cold active RNA polymerase transcription template, firefly luciferase suitable for transfection of mammalian cells.

SEQ ID NO:92 is an example T7 RNA polymerase transcription template, firefly luciferase suitable for transfection of mammalian cells.

### DETAILED DESCRIPTION

Since its discovery over fifty years ago, the RNA polymerase from Escherichia phage T7 (T7 RNA polymerase) has played an outsized role in molecular biology. Owing to its high transcriptional activity, stringent promoter specificity, and ease of use as a single subunit enzyme, T7 RNA polymerase has enabled technologies which include recombinant protein expression, in vitro transcription and translation, genetic circuitry, molecular diagnostics, and single-cell whole-genome sequencing methods, among others. T7 RNA polymerase is also the principal enzymatic tool used in the manufacturing of mRNA drugs.

A key limitation to the efficacy of mRNA therapeutics is off-target immune stimulation caused by recognition of the delivered nucleic acid as foreign. Despite the benefits of nucleotide modification, unwanted immunostimulatory species formed in the IVT reaction remain a barrier to the development of mRNA drugs beyond immunotherapies. The problematic reaction byproducts may include degraded or uncapped RNA molecules, or double-stranded RNA (dsRNA) species. dsRNA activates cellular sensors which recognize viral genomes and may be the principle immunostimulatory species present in mRNA therapeutic formulations. Two mechanisms by which T7 RNA polymerase produces dsRNA have been elucidated. In one mechanism, the run-off transcript folds back on itself to prime the 3′ end and is extended by the weak RNA-dependent RNA polymerase activity of T7 RNA polymerase. In another mechanism, T7 RNA polymerase initiates transcription at the terminus of the linear DNA template lacking a T7 promoter to produce a fully complementary anti-sense RNA molecule. Production of dsRNA species during IVT can be minimized by conducting IVT at elevated temperatures or in the presence of chaotropes to disfavor RNA self-annealing, or the desired single-stranded RNA (ssRNA) can be purified from byproducts by gel electrophoresis or chromatography. These methods are imperfect and may be difficult to scale up. New RNA polymerases which produce a higher quality drug substance with less required purification steps are desirable to help realize the full potential of this drug modality.

T7 RNA polymerase has been extensively investigated and engineered; mutations to T7 RNA polymerase that confer thermotolerance, reduce abortive transcription, alter promoter specificity, or result in lower levels of dsRNA in IVT have been identified. The residue Ser43 was implicated as a determinant of dsRNA production by two independent groups who took distinct approaches, a result that highlights the limited sequence space which can be explored by mutating T7 RNA polymerase. Moreover, T7 RNA polymerase activity depends on the initially transcribed sequence and directing evolution on a single DNA template may produce enzymes which are specifically adapted for that sequence. An alternative approach to the discovery of improved RNA polymerases is exploring natural homologs of T7 RNA polymerase found in the Autographiviridae (self-transcribing) family of bacteriophages. Salmonella phage SP6 RNA polymerase has served for decades as a comparison to T7 RNA polymerase and synthesizes RNA with similarly high yield, but also produces high levels of immunostimulatory contaminants. Polymerases from Synechococcus phage Syn5, Klebsiella phage KP34 and Pseudomonas phage VSW-3 have some advantages compared to T7 RNA polymerase including higher processivity, lower temperature optima, and reduced byproduct synthesis, but all suffer from low RNA yield.

The present disclosure relates, in some embodiments, compositions, kits, methods, workflows, and systems for the synthesis of RNA molecules. For example, RNA molecules (e.g., therapeutic mRNA molecules) may be synthesized using the RNA polymerase derived from bacteriophage T7. Rapid growth in the field of mRNA therapies motivates engineering and discovery of novel RNA polymerases which have specific advantages for mRNA manufacturing. The present disclosure provides, in some embodiments, cold-active RNA polymerases including, for example, an RNA polymerase from Pseudomonas phage Njord. Cold-active RNA polymerases may be derived from a viral subfamily adapted for lytic infection of marine bacteria (e.g., Colwellvirinae). These polymerases may synthesize RNA at yields comparable to SP6 and T7 RNA polymerases but with peak activity at a temperature that is ≥5° C., ≥10° C., ≥12° C., ≥15° C., ≥18° C., or ≥20° C. lower than the peak activity of wildtype SP6 or T7.

According to some embodiments, a cold-active RNA polymerase may synthesize RNA with high fidelity (e.g., higher fidelity than wild type T7 RNA polymerase). A cold-active RNA polymerase may have the capacity to initiate RNA synthesis from a 5′ cap analog oligonucleotide, in some embodiments. A cold-active RNA polymerase may synthesize RNA molecules, in some embodiments, comprising fewer immunostimulatory double-stranded RNA molecules than a reference RNA polymerase (e.g., T7 RNA polymerase). For example, RNA synthesis (e.g., IVT) with a cold-active RNA polymerase may result in formation of fewer long double-stranded byproducts than a reference RNA polymerase (e.g., T7 RNA polymerase) as reflected by reduced interferon induction in cell culture. RNA synthesis (e.g., IVT) with a cold-active RNA polymerase may constitute a useful alternative to T7 RNA polymerase when reduced temperature during transcription is desired, for example in the synthesis of self-amplifying mRNA vaccines. Liquid RNA synthesis reactions comprising an RNA polymerase from Pseudomonas phage Njord (and/or other cold-active RNA polymerases) may yield high-quality mRNAs at yields comparable to T7 polymerase, but at temperatures≤35° C., ≤30° C., ≤25° C., ≤20° C., ≤15° C., ≤10° C., and/or ≤5° C. Activity at lower temperatures may be desirable, for example, in reactions for synthesizing saRNA vaccines.

Synthetic RNA for use as a therapeutic drug substance may be capped at its 5′ terminus with a N7-methylguanosine residue via a triphosphate linkage. This ‘mRNA cap’ is conserved in the biology of higher eukaryotes. RNA produced by a bacteriophage RNA polymerase bears a 5′ triphosphate moiety and cannot be translated by eukaryotic ribosomes. Thus, additional measures must be taken to convert the RNA 5′ triphosphate group to the N7-methylguanosine cap, and additional methylation events may further improve its quality as a drug substance. A method of producing capped RNA with bacteriophage RNA polymerases may include using synthetic oligonucleotides to initiate transcription. In some embodiments, a method of producing capped RNA may include using capping enzymes derived from viruses of eukaryotes to post-transcriptionally cap the RNA. A challenge in manufacturing mRNA therapies is the inability of T7 RNA polymerase to perform co-transcriptional capping with vaccinia capping enzyme. An RNA polymerase that can function in concert with a capping enzyme and/or which performs well when fused to a capping enzyme may facilitate the synthesis of capped mRNA therapies. Thus, a desirable RNA polymerase for mRNA synthesis would both produce low levels of dsRNA and facilitate co-transcriptional mRNA capping.

### General Considerations

Aspects of the present disclosure can be understood in light of the provided descriptions, figures, sequences, embodiments, section headings, and examples, none of which should be construed as limiting the entire scope of the present disclosure in any way. Accordingly, the innovations set forth herein should be construed in view of the full breadth and spirit of the disclosure.

Each of the individual embodiments described and illustrated herein has discrete components and features which can be readily separated from or combined with the components and/or features of any of the other several embodiments without departing from the scope or spirit of the present teachings. Lists of example species within a particular genus may vary in length at different places throughout the disclosure. Species lists shortened for convenience shall not be construed to exclude example species listed elsewhere in the specification. Any recited method can be carried out in the order of events recited or in any other order which is logically possible. Unless otherwise expressly stated to be required herein, each component, feature, and method step disclosed herein is optional and the disclosure contemplates embodiments in which each optional element may be expressly excluded. As such, this statement is intended to serve as antecedent basis for use of such exclusive terminology as “solely,” “only” and the like in connection with the recitation of claim elements or use of a “negative” limitation. It is further intended to serve as antecedent basis for use of such elective terminology as “optionally” and the like in connection with the recitation of one or more claim elements.

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and ease of reference.

Sources of commonly understood terms and symbols may include: standard treatises and texts such as Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like.

As used herein and in the appended claims, the singular forms “a” and “an” include plural referents unless the context clearly dictates otherwise. For example, the term “a protein” refers to one or more proteins, i.e., a single protein and multiple proteins.

Numeric ranges are inclusive of the numbers defining the range. All numbers should be understood to encompass the midpoint of the integer above and below the integer i.e., the number 2 encompasses 1.5-2.5. The number 2.5 encompasses 2.45-2.55 etc. When sample numerical values are provided, each alone may represent an intermediate value in a range of values and together may represent the extremes of a range unless specified. Percent ranges with only one end point (e.g., ≥90% or ≤10%) optionally include a second endpoint at the maximum or minimum percentage (e.g., ≥90% includes a range of 90%-100% and ≤10% includes a range of 0%-10%). Ranges (including percent ranges) with only one end point (e.g., ≥90 or ≤10) optionally include a second endpoint 10% higher or 10% lower than the provided endpoint (e.g., ≥90 includes a range of 90-99 and ≤10 includes a range of 1-10). Concentration percentages are w/v percentages unless otherwise indicated.

In the context of the present disclosure, “artificial transcript” refers to a non-naturally occurring transcript that is translatable in a mammalian cell (e.g., to form a translation product) and/or heterologous to such mammalian cell. In some embodiments, a translation product may also be heterologous to mammalian cells. In some embodiments, the mammalian cell is a human cell. Example artificial transcripts include therapeutic RNAs and vaccines. An artificial transcript may be encoded by an artificial coding sequence. An artificial RNA transcript may differ from naturally-occurring RNA transcripts, for example, in that it comprises one or more modified nucleotides, it comprises a non-naturally occurring nucleotide sequence (e.g., designed by a computer algorithm or artificial intelligence), and/or it is bound (e.g., covalently) to a support (e.g., immobilized), a dye, fluorophore or other label, a protein (e.g., enzyme), an additional RNA molecule to form a branched structure, and/or or a carbohydrate.

In the context of the present disclosure, “buffer” and “buffering agent” refer to a chemical entity or composition that itself resists and, when present in a solution, allows such solution to resist changes in pH when such solution is contacted with a chemical entity or composition having a higher or lower pH (e.g., an acid or alkali). Examples of suitable non-naturally occurring buffering agents that may be used in disclosed compositions, kits, and methods include HEPES, MES, MOPS, TAPS, tricine, and Tris. Additional examples of suitable buffering agents that may be used in disclosed compositions, kits, and methods include ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, and triethanolamine.

In the context of the present disclosure, “cap” refers to a natural polyribonucleotide cap (e.g., ⁷ mG) and to a compound of the general formula R3p₃N1-[p-N](x), where R3 is a guanine, adenine, cytosine, uridine or analogs thereof (e.g., N⁷-methylguanosine; m⁷G), p₃ is a triphosphate linkage, N1 and Nx are ribonucleosides, x is 0-8 and p is, independently for each position, a phosphate group, a phosphorothioate, a phosphorodithioate, an alkylphosphonate, an arylphosphonate, or a N-phosphoramidate linkage. R3 may have an added label at the 2′ or 3′ position of the ribose, and, in some embodiments, the label may be an oligonucleotide, a detectable label such as a fluorophore, or a capture moiety such as biotin or desthiobiotin, where the label may be optionally linked to the ribose of the nucleotide by a linker, for example. See, e.g., WO 2015/085142. A cap may have a cap 0 structure, a cap 1 structure or a cap 2 structure (e.g., as reviewed in Ramanathan, Nucleic Acids Res. 2016 44:7511-7526), depending on which enzymes and/or whether SAM is present in the capping reaction.

Caps include dinucleotide cap analogs, e.g., of formula m⁷G (5′) p₃ (5′) G, in which a guanine nucleotide (G) is linked via its 5′OH to the triphosphate bridge. In some dinucleotide caps the 3′—OH group is replaced with hydrogen or OCH₃ (U.S. Pat. No. 7,074,596; Kore, Nucleosides, Nucleotides, and Nucleic Acids, 2006, 25:15 307-14; and Kore, Nucleosides, Nucleotides, and Nucleic Acids, 2006, 25:337-40). Dinucleotide caps include m⁷G (5′) p₃G, 3′-OMe-m⁷G (5′) p₃G (ARCA). Caps also include trinucleotide cap analogs (defined below) as well as other, longer, molecules (e.g., cap that have four, five or six or more nucleotides joined to the triphosphate bridge). In a cap analog, the 2′ and 3′ groups on the ribose of the m⁷G may be independently selected O-alkyl (e.g., O-methyl), halogen, a linker, hydrogen or a hydroxyl and the sugars 20 in N1 and NX may be independently selected from ribose, deoxyribose, 2′-O-alkyl, 2′-O-methoxyethyl, 2′-O-allyl, 2′-O-alkylamine, 2′-fluororibose, and 2′-deoxyribose. N1 and NX may independently (for each position) comprise a base selected from adenine, uridine, guanine, or cytidine or analogs of adenine, uridine, guanine, or cytidine, and nucleotide modifications can be selected from N⁶-methyladenine, N¹-methyladenine, N⁶-2′-Odimethyladenosine, pseudouridine, N¹-methylpseudouridine, 5-iodouridine, 4-thiouridine, 2-thiouridine, 5-methyluridine, pseudoisocytosine, 5-methoxycytosine, 2-thiocytosine, 5-hydroxycytosine, N⁴-methylcytosine, 5-hydroxymethylcytosine, hypoxanthine, N1-methylguanine, O⁶-methylguanine, 1-methyl-guanosine, N²-methylguanosine, N²,N²-dimethyl-guanosine, 2-methyl-2′-O-methyl-guanosine, N²,N²-dimethyl-2′-O-methyl-guanosine, 1-methyl-2′-O-methyl-guanosine, N²,N⁷-dimethyl-2′-O-methyl-guanosine, and isoguanineadenine.

In the context of the present disclosure, “capping” refers to the enzymatic addition of a Nppp-moiety onto the 5′ end of an RNA, where N a nucleotide such as G or a modified G. A modified G may have a methyl group at the N7 position of the guanine ring, or an added label at the 2 or 3 position of the ribose, and, in some embodiments, the label may be an oligonucleotide, a detectable label such as a fluorophore, or a capture moiety such as biotin or desthiobiotin, where the label may be optionally linked to the ribose of the nucleotide by a linker, for example. See, e.g., WO 2015/085142. A cap may have a Cap-0 structure, a Cap-1 structure or a cap 2 structure (as reviewed in Ramanathan, Nucleic Acids Res. 2016 44:7511-7526), depending on which enzymes are included (e.g., a 2′ O-methyl transferase) and/or whether SAM is present in the capping reaction.

In the context of the present disclosure, “capping enzyme” refers to an enzyme operable to cap RNA. A capping enzyme may be referred to as a single-chain capping enzyme if it consists of a single polypeptide chain that alone has detectable RNA triphosphatase (TPase), guanylyltransferase (GTase), and guanine-N7 methyltransferase (N7 MTase) activities (e.g., without the necessity of forming a dimer with another polypeptide chain). Faustovirus, mimivirus and moumouvirus capping enzymes are examples of single-chain RNA capping enzymes. Naturally occurring vaccinia capping enzyme (VCE) is a heterodimer and, as such, is not a single-chain RNA capping enzyme. Examples of capping enzymes may be found in US20210054016, U.S. Pat. No. 11,028,379, and US20230287376. Faustovirus capping enzymes include, for example, RNA capping enzymes of Faustovirus D5b, Faustovirus E12, Faustovirus ST1, and Faustovirus LC9. A single-chain capping enzyme, according to some embodiments, may have ≥75%, ≥80%, ≥85%, ≥90%, ≥92%, ≥94%, ≥95%, ≥98%, or ≥99% identity to any of SEQ ID NOS: 75-80.

In the context of the present disclosure, “cold-active RNA polymerase” refers to an enzyme that catalyzes template-dependent, 5′ to 3′ synthesis of RNA with peak catalytic activity at temperatures in ranges X to Y, where X is any of 18° C., 19° C., 20° C., 21° C., 22° C., 23° C., 24° C., 25° C., Y is any of 21° C., 22° C., 23° C., 24° C., 25° C., 26° C., 27° C., 28° C., 29° C., 30° C., 31° C., 32° C., and X<Y. For example, a cold-active RNA polymerase may have its peak catalytic activity at temperatures in ranges 18° C.-32° C., 20° C.-30° C., 22° C.-28° C. or 24° C.-28° C. Example cold-active RNA polymerases include Pseudomonas phage Njord (SEQ ID NO:1), Pseudomonas phage Nerthus (SEQ ID NO:2), Pseudomonas phage Alpheus (SEQ ID NO:3), Pseudomonas phage Achelous (SEQ ID NO:4), Pseudomonas phage uligo (SEQ ID NO:5), Vibrio phage qA318 (SEQ ID NO:6), Vibrio phage qAS51 (SEQ ID NO:7), Vibrio phage Vp670 (SEQ ID NO:8), Vibrio phage Vc1 (SEQ ID NO:9), Vibrio phage VEN (SEQ ID NO: 10), Marinomonas phage CPP1m (SEQ ID NO:11), Marinomonas phage CB5A (SEQ ID NO: 12), Pseudomonas phage Ulina01 (SEQ ID NO:13), Pseudomonas phage Ulitu01 (SEQ ID NO: 14), and Pseudomonas phage BUCT553 (SEQ ID NO:15). A cold-active RNA polymerase may have catalytic activity at temperatures in ranges X to Y, where X is any of 1° C., 2° C., 3° C., 4° C., 5° C., 6° C., 7° C., 8° C., 10° C., 15° C., 20° C., 24° C., 25° C., 28° C. and Y is any of 5° C., 10° C., 15° C., 20° C., 24° C., 25° C., 28° C., 30° C., 32° C., 35° C. and X<Y. For example, a cold-active RNA polymerase may have catalytic activity at temperatures in ranges 4° C.-35° C., 8° C.-32° C., 10° C.-37° C., 15° C.-35° C., 12° C.-32° C., 16° C.-32° C., 20° C.-32° C., 18° C.-30° C., or 20° C.-30° C.

Activity of a cold-active RNA polymerase may be expressed in terms of units where a unit of a cold-active RNA polymerase is the amount of enzyme needed to catalyze synthesis of over 10 micrograms of RNA from 1 microgram on DNA template (having a promoter matched to the polymerase) in the presence of 5 millimolar of each nucleoside triphosphate at 25° C. (e.g., in a volume of 20 μL). Specific activity of a cold-active RNA polymerase may be expressed in terms of units per milligram of cold-active RNA polymerase protein. Substrate specificity of a cold-active RNA polymerase may be expressed in terms of the probability of achieving a unit of activity on a random sequence of 1000 nucleotides of DNA and this probability may be less than 1 in 107.

A cold-active RNA polymerase may have an amino acid sequence having ≥75%, ≥80%, ≥85%, ≥90%, ≥92%, ≥94%, ≥95%, ≥98%, or ≥99% identity to any of SEQ ID NOS: 1-19, optionally wherein ≤25, ≤20, ≤15, ≤10, ≤9, ≤8, ≤7, ≤6, ≤5, ≤4, ≤3, or ≤2 of the amino acids differ from SEQ ID NOS: 1-15. In some embodiments, a cold-active RNA polymerase may have an amino acid sequence that is at least 90% identical to one or more of SEQ ID NOS: 1-16 with any/all variations from the reference sequence (e.g., SEQ ID NO:1-16) at non-conserved positions. For example, conserved positions of a cold-active RNA polymerase may include all positions (other than those represented as “Xaa” of SEQ ID NO:17. In this light, a cold-active RNA polymerase may have an amino acid sequence that is ≥90%, ≥92%, ≥95%, ≥97%, or ≥98% identical to one (or more) of SEQ ID NOS: 1-16 and 100% identical to SEQ ID NO: 17. A non-naturally occurring cold-active RNA polymerase may have an amino acid sequence having less than 100% identity with all of SEQ ID NOS: 1-15 (but having ≥90%, ≥92%, ≥95%, ≥97%, or ≥98% identity to one (or more) of SEQ ID NOS: 1-16) and 100% identity with SEQ ID NO: 17.

A cold-active RNA polymerase may comprise one or more amino acids in addition to a corresponding wild type enzyme. For example, a cold-active RNA polymerase (e.g., SEQ ID NOS: 1-19) may comprise (e.g., at its amino terminal end or carboxy terminal end) 1-25 additional amino acids. Such additional amino acids may enable, facilitate and/or enhance translation, expression, cellular sorting, inactivation (e.g., by including a protease recognition and/or cleavage site), and/or purification. Such additional amino acids may constitute a linker, for example, to a support (e.g., a magnetic bead) or another protein.

A cold-active RNA polymerase may catalyze template-dependent, 5′ to 3′ synthesis of RNA, which RNA (a) may be less immunostimulatory than an RNA having the same sequence but synthesized by T7 RNA polymerase (e.g., at 37° C.) or another RNA polymerase (e.g., at its optimal temperature) and/or (b) may comprise less dsRNA than an RNA having the same sequence but synthesized by T7 RNA polymerase (e.g., at 37° C.) or another RNA polymerase (e.g., at its optimal temperature).

In the context of the present disclosure, “container” refers to a human-made container. A container may comprise one or more walls (e.g., defining an interior volume) and optionally one or more openings. Containers comprising one or more openings may further comprise one or more closures (e.g., a removable closures) for some or all such openings. A closure optionally may comprise an aperture or a septum, for example, to provide fluid communication with a volume of the container and a connected or inserted tube or syringe. Examples of containers include boxes, cartons, bottles, tubes (e.g., test tubes, microcentrifuge tubes), plates (e.g., 96-well, 384-well plates), vials, pipette tips, and ampules. Containers and/or closures may comprise any desired material including paper, plastics, glass, silicone, composites, metals, alloys, or combinations thereof. Containers and/or closures may comprise materials that are compostable, recyclable, and/or sustainable.

In the context of the present disclosure and with respect to an amino acid residue or a nucleotide base position, “corresponding to” refers to positions that lie across from one another when sequences are aligned, e.g., by the BLAST algorithm. An amino acid position in a functional or structural motif in one polymerase may correspond to a position within a functionally equivalent functional or structural motif in another polymerase.

In the context of the present disclosure, “elicitor transcript” refers to an RNA transcript operable to impact a cell, tissue, organ or organism in which is introduced or made. For example, an elicitor transcript may directly or through a translation product have a therapeutic effect on a cell, tissue, organ or organism. A therapeutic effect may be direct (e.g., where the translation product is a functional version of a protein that is defective or missing from the cell, tissue, organ or organism) or indirect (e.g., where the translation product is cytotoxic or catalyzes formation of a product that is cytotoxic to a malignant cell or otherwise undesirable cell in which it is produced, but remaining cells, tissues, organs, and/or the organism benefits from targeted removal of the unwanted cells).

In the context of the present disclosure, “expression system” refers to systems for producing a protein from a polynucleotide template comprising components to produce the protein according to an RNA template (e.g., enzymes, amino acids, an energy source), (optionally) components to produce the RNA template according to another RNA template or a DNA template (e.g., enzymes, nucleotides, an energy source). An expression system may comprise a bacterial (e.g., Escherichia coli) or yeast (e.g., Kluyveromyces lactis or Pichia pastoris) expression system in which the protein is encoded by an RNA or DNA template within an expression cassette, a plasmid or other expression vector. An expression system may comprise a viral expression system in which the protein is encoded by an RNA or DNA template (e.g., in an expression cassette) within a viral genome or viral expression vector. Examples of cell-free expression systems may include or comprise cell extracts of Escherichia coli S30, rabbit reticulocytes or wheat germ, PUREEXPRESS® (New England Biolabs, Ipswich, MA), an insect cell extract system (e.g., Promega #L1101), or HeLa cell lysate-based protein expression systems (e.g., Thermo Fisher Scientific #88882). An expression cassette may comprise, in some embodiments, an expression control sequence (e.g., promoter), a coding sequence encoding the gene product (e.g., protein) of interest (e.g., a vaccinia capping enzyme fusion), and/or one or more termination sequences (e.g., terminators). An expression control sequence (e.g., promoter) may comprise any promoter operative in a desired expression system, including, for example, a GAP promoter, an AOX1 promoter, a LAC4 promoter, a P350 hybrid promoter, a T7 promoter, a T5 promoter, a Ptac promoter, a Ptrc promoter, ParaBAD promoter, a PrhaBAD promoter, a Tet promoter or a PhoA phosphate-starvation promoter.

In the context of the present disclosure, “fusion” refers to two or more polypeptides, subunits, or proteins covalently joined to one another (e.g., by a peptide bond). For example, a protein fusion may refer to a non-naturally occurring polypeptide comprising a protein of interest covalently joined to a second polypeptide. Examples of a second polypeptide include a reporter protein (e.g., a green fluorescent protein), a purification tag, and expression tag, a polynucleotide binding protein, an enzyme (e.g., a capping enzyme), a conjugation tag (e.g., a SNAP® tag), and a peptide linker (e.g., a flexible linker, an inflexible linker, a cleavable linker). Unless otherwise disclosed, the protein of interest may be nearer to the N-terminal end or nearer to the C-terminal end than the second polypeptide to which it is joined. For example, a fusion may comprise a cold-active RNA polymerase fused at its N-terminal or C-terminal end to a capping enzyme. A fusion may comprise a non-naturally occurring combined polypeptide chain comprising two proteins or two protein domains joined directly to each other by a peptide bond or joined through a peptide linker. In some embodiments, a fusion may comprise a cold-active RNA polymerase (e.g., a wt or variant cold-active RNA polymerase) covalently joined to a second polypeptide. In some embodiments, a variant cold-active RNA polymerase may include a fusion to an exogenous DNA binding domain. Examples are provided in Table 1 of U.S. Pat. No. 11,259,184. In some embodiments, a fusion may comprise a cold-active RNA polymerase covalently joined to a second polypeptide (e.g., a capping enzyme). A fusion, in some embodiments, may comprise a cold-active RNA polymerase operably linked to a capping enzyme wherein both enzymes are catalytically active. For example, a cold-active RNA polymerase and a capping enzyme may be directly joined or may be joined by a peptide linker. For clarity, the temperature of peak activity and the peak activity itself may differ for a given polyermase and/or capping enzyme in the context of a fusion protein relative to the temperature and peak activity of the enzymes outside the context of the fusion (e.g., as a standalone enzyme).

In the context of the present disclosure, “immobilized” refers to covalent attachment of an enzyme (e.g., a cold-active RNA polymerase, a capping enzyme) to a solid support with or without a linker. Examples of solid supports include beads (e.g., magnetic, agarose, polystyrene, polyacrylamide, chitin). Beads may include one or more surface modifications (e.g., O⁶-benzyleguanine, polyethylene glycol) that facilitate covalent attachment and/or activity of an enzyme of interest. For example, a support may comprise a ligand and an enzyme may have a receptor for such ligand or an enzyme may comprise a ligand and a support may comprise a receptor for such ligand. Receptor-ligand binding may be covalent or non-covalent. Non-covalent attachment (e.g., avidin: biotin, chitin: CBP) may be useful in some embodiments, for example, where the level of dissociation of the binding partner is deemed tolerable. A linker may be disposed between a support and an enzyme. For example, linker disposed between a support and an enzyme may have a first covalent bond to the support and a second covalent bond to the enzyme. An immobilized enzyme comprising a ligand-receptor attachment may have a linker disposed between the support and the ligand-receptor attachment, a linker disposed between the enzyme and the ligand-receptor attachment, or both. An immobilized enzyme comprising a linker may also comprise an optional covalent bond directly between the enzyme and the support. A linker may be of any desired length and have any desired range of motion. A peptide linker may comprise one or more repeats (e.g., 1-10 repeats) of glycine-serine.

In the context of the present disclosure, “in vitro transcription” (IVT) refers to a cell-free reaction in which a DNA template is copied by a DNA-directed RNA polymerase (e.g., a cold-active RNA polymerase) to produce a product that comprises one or more RNA molecules having a sequence copied from the template. For clarity, IVT optionally may include co-transcriptional capping.

In the context of the present disclosure, “IVT fusion” refers to an enzyme comprising a polymerase (e.g., a cold-active RNA polymerase) and a capping enzyme (e.g., a Faustovirus capping enzyme).

In the context of the present disclosure, “modified nucleotide” refers to nucleotides having a modification on the sugars (e.g., 2′-fluororibose, ribose, 2′-deoxyribose, arabinose, and hexose); and/or in the phosphate groups (e.g., phosphorothioates and 5′-N-phosphoramidite linkages); and/or in the nucleotide base (e.g., as described in U.S. Pat. No. 8,383,340; WO 2013/151666; U.S. Pat. No. 9,428,535 B2; US 2016/0032316). Examples of modified nucleotides include pseudouridine and N1-methyl-pseudouridine.

In the context of the present disclosure, “non-naturally occurring” refers to a molecule (e.g., a polynucleotide, polypeptide, carbohydrate, or lipid) or composition that does not exist in nature. Such a molecule or composition may differ from naturally occurring molecules or compositions in one or more respects. For example, a polymer (e.g., a polynucleotide, polypeptide, or carbohydrate) may differ in the kind and arrangement of the component parts (e.g., nucleotide sequence, amino acid sequence, or sugar molecules). A polymer may differ from a naturally occurring polymer with respect to the molecule(s) to which it is linked. For example, a “non-naturally occurring” polypeptide (e.g., protein) may differ from naturally occurring polypeptides in its secondary, tertiary, or quaternary structure, by having (or lacking) a chemical bond (e.g., a covalent bond including a peptide bond, a phosphate bond, a disulfide bond, an ester bond, and ether bond, and others) to a lipid, a carbohydrate, a second polypeptide (e.g., a fusion protein), or any other molecule. Similarly, a “non-naturally occurring” polynucleotide or nucleic acid may comprise (or lack) one or more other modifications (e.g., an added label or other moiety) to the 5′-end, the 3′ end, and/or between the 5′- and 3′-ends (e.g., methylation) of the nucleic acid. A “non-naturally occurring” molecule or composition may differ from naturally occurring compositions in one or more of the following respects: (a) having components that are not combined in nature, (b) having components in ratios and/or concentrations not found in nature, (c) lacking one or more components otherwise found in naturally occurring molecules or compositions (e.g., a cell-free composition, a chromosome-free composition, a histone-free composition, a polymerase-free composition, a cell membrane-free composition), (d) having a form not found in nature (e.g., dried, freeze dried, lyophilized, crystalline, aqueous, immobilized), and (e) having one or more additional components beyond those found in nature (e.g., a buffering agent, a detergent, a dye, a solvent or a preservative).

In the context of the present disclosure, “polymerase” refers to an enzyme that synthesizes a polyribonucleotide from NTPs with or without a template. Examples of polymerases include T3 RNA polymerase, T7 RNA polymerase, SP6 polymerase, cold-active RNA polymerases, among others and variants thereof including psychrophilic, mesophilic, and/or thermostable variants (e.g., International PCT Publication No. WO2017123748 and U.S. Pat. Nos. 10,519,431 and 11,259,184).

With reference to an amino acid, “position” refers to the place such amino acid occupies in the primary sequence of a peptide or polypeptide numbered from its amino terminus to its carboxy terminus.

In the context of the present disclosure, “substitution” refers to an amino acid residue at a position in a comparator amino acid sequence that differs with respect to a corresponding position of a reference amino acid sequence, where the comparator and reference sequences are at least 60% identical to each other or at least 70% identical to each other or at least 80% identical to each other. A reference sequence and comparator sequence may have the same length or similar lengths (e.g., differing by ≤12%, ≤5%, ≤1%). A substitute amino acid residue at a position, in addition to differing from the corresponding position of a reference amino acid sequence, may differ from the amino acid at the corresponding position of all naturally-occurring sequences that are at least 60% identical to each other or at least 70% identical to each other or at least 80% identical to the reference sequence. Optionally, a substitute amino acid may have different properties than the amino acid in the corresponding position of the reference sequence. Optionally, a substitute amino acid may have similar properties to the amino acid in the corresponding position of the reference sequence (a “conservative” substitution). For example, a non-polar amino acid (e.g., A, V, L, I, M, W, and F (and optionally C, G, and P) may substitute for another non-polar amino acid, a polar amino acid (e.g., N, Q, S, T, and Y) may substitute for another polar amino acid (e.g., C, D, E, H, K, N, P. Q, R, S, and T), a positively charged amino acid (H, K, and R) may substitute for another positively charged amino acid, and a negatively charged amino acid (e.g., D and E) may substitute for another negatively charged amino acid. A substitute amino acid may be a natural amino acid (e.g., replacing another natural amino acid or a non-natural amino acid). A substitute amino acid may be a non-natural amino acid (e.g., replacing a natural amino acid or another non-natural amino acid).

In the context of the present disclosure, “transcript” refers to a polyribonucleotide template encoding a polypeptide. A transcript may comprise RNA (e.g., ssRNA), a cap or cap analog, and/or a polyA tail. A transcript may be capable of translation in a cell (e.g., a bacterial cell and/or a yeast cell). For example, a transcript may be or comprise mRNA. A fusion transcript may comprise polynucleotide templates for two or more polypeptides in a single polynucleotide. A transcript may comprise or consist of a single strand polynucleotide (e.g., having few or no hairpins, internal loops, bulge loops, or other double-stranded portions). For example, a transcript formed by a cold-active RNA polymerase may have fewer and/or shorter double-stranded portions than a transcript having the same sequence formed by T7 RNA polymerase.

In the context of the present disclosure, “uncapped” refers to an RNA (a) that does not have a cap and (b) that can be used as a substrate for a capping enzyme. Uncapped RNA typically has a tri- or di-phosphorylated 5′ end. RNAs transcribed in vitro have a triphosphate group at the 5′ end.

All publications, patents, and patent applications mentioned in this specification are herein incorporated by reference to the same extent as if each individual publication, patent, or patent application was specifically and individually indicated to be incorporated by reference. Reagents referenced in this disclosure may be made using available materials and techniques, obtained from the indicated source, and/or obtained from New England Biolabs, Inc. (Ipswich, MA).

### Enzymes and Compositions

The present disclosure relates, in some embodiments, to cold-active RNA polymerases (including variant cold-active RNA polymerases) having one or more desirable properties including, for example, efficient and cold synthesis of RNA relative to, for example, wildtype T7 RNA protease.

According to some embodiments, a cold-active RNA polymerase composition may comprise a cold-active RNA polymerase (e.g., a wildtype or variant cold-active RNA polymerase) and, optionally, any of (including one or more of) a buffering agent (e.g., a storage buffer, a reaction buffer), an excipient, a salt (e.g., NaCl, MgCl₂, CaCl₂)), a protein (e.g., a capping enzyme, a 2′ O-methyl transferase, another enzyme or protein), a stabilizer, a detergent (for example, ionic, non-ionic, and/or zwitterionic detergents (e.g., octoxinol, polysorbate 20)), a polyanion (e.g., spermidine, spermine, putrescine), a polynucleotide (e.g., a template comprising a sequence encoding an RNA of interest and optionally a cold-active RNA polymerase promoter), a cell (e.g., intact, digested, or any cell-free extract), a biological fluid or secretion (e.g., mucus, pus, blood, urine, saliva), an aptamer, a pH indicator (e.g., azolitimin, bromocresol purple, bromothymol blue, methylene blue, cresol red, neutral red, naphtholphthalein, phenol red), a crowding agent, a sugar (e.g., a mono, di, tri, tetra, or higher saccharide), a starch, cellulose, a glass-forming agent (e.g., glycerol, raffinose, stachyose, or trehalose for lyophilization), a lipid, an oil, aqueous media, a support (e.g., a bead) and/or (non-naturally occurring) combinations thereof. Combinations may include for example, two or more of the listed components (e.g., a salt and a buffer) or a plurality of species of a single listed component (e.g., two different salts or two different sugars). In some embodiments, a composition may comprise 0.5-25 mM MgCl₂, e.g., 2 mM MgCl₂. Compositions may comprise one or more polyanions at any desired concentration (e.g., individually or total concentrations of 0.1-10 mM, 0.5-5 mM, 1 mM, 1.5 mM, 2 mM, 2.5 mM, 3 mM, 3.5 mM, 4 mM, 4.5 mM or 5 mM) and may be included to bind negatively charged molecules. Example polyanions include spermidine, spermine, putrescine, polyethylenimine, 1,4,7-triazacyclononane, cyclen, ethylenediamine, or 1, 3, 5,-triazinane. According to some embodiments, cold-active RNA polymerase compositions may comprise (a) a cold-active RNA polymerase (e.g., a wt or variant cold-active RNA polymerase), (b) a buffer, and optionally (c) a polynucleotide (e.g., a template DNA) or a cellular extract or a cell-free preparation comprising a polynucleotide.

Cold-active RNA polymerases and variant cold-active RNA polymerases described herein have RNA polymerase activity and, as such, have the capacity to catalyze the formation of RNA in the 5′-≥3′ direction using a DNA template. A DNA template may comprise a suitable promoter (e.g., a sequence having ≥85%, ≥90% or 100% identity to any of SEQ ID NOS: 31-46).

A cold-active RNA polymerase composition may comprise, for example, a variant cold-active RNA polymerase (e.g., having an amino acid sequence at least 85% identical to SEQ ID NO:1) and having at least one substitution, deletion, or insertion relative to wildtype cold-active RNA polymerase. A cold-active RNA polymerase composition may be free of one or more other catalytic activities. For example, a cold-active RNA polymerase (e.g., a wt or variant cold-active RNA polymerase) may be free of proteases (e.g., non-specific proteases or proteases having other cleavage recognition sites), free of nucleases (e.g., RNases and/or DNases), free of other polymerase activity, free of RNA and/or DNA modification activity, free of kinase activity, and/or free of phosphorylation and/or glycosylation activities, in each case, under desired test conditions (e.g., conditions of time, temperature, pH, salinity, model or intended substrate and/or others), for example, conditions intended to replicate conditions of a specific use of the cold-active RNA polymerase composition or intended to represent conditions for a range of uses.

The present disclosure relates, in some embodiments, to an immobilized enzyme comprising a support and an enzyme immobilized thereto. For example, an immobilized cold-active RNA polymerase may comprise a cold-active RNA polymerase (e.g., a wt or variant cold-active RNA polymerase), a glycine-serine linker attached to the variant cold-active RNA polymerase by a peptide bond, a protein tag (e.g., a SNAP-tag) attached to the linker by a peptide bond, 06-benzyleguanine bound to the protein tag (e.g., SNAP-tag®); and beads (e.g., magnetic beads) having a surface modification comprising the 06-benzyleguanine. In some embodiments, a support of an immobilized cold-active RNA polymerase (e.g., an immobilized variant cold-active RNA polymerase) may comprise a magnetic bead. A magnetic bead may comprise, for example, one or more surface modifications. Surface modifications may include, for example, 06-benzyleguanine and/or PEG750. In some embodiments, an immobilized enzyme may comprise a ligand (e.g., 06-benzyleguanine) and a receptor or tag (e.g., a SNAP-tag®) capable of binding the ligand. For example, ligands may be disposed on a support and corresponding receptors may be disposed on (e.g., covalently attached to) an enzyme to be immobilized on the support. An immobilized enzyme may comprise, in some embodiments, an enzyme (e.g., variant cold-active RNA polymerase), optionally, a first linker (e.g., a peptide linker) attached to the enzyme, a polypeptide tag (e.g., a SNAP-tag®) attached to the first linker, if present, or the enzyme, a ligand corresponding to the polypeptide tag (e.g., 06-benzyleguanine) attached (e.g., covalently attached) to the tag, optionally, a second linker (e.g., polyethylene glycol) attached to the ligand, and a support (e.g., a magnetic bead) attached to the second linker if present or the ligand, the structure of which may be illustrated, in an N-≥C direction, as:
ENZYME-[LINKER-]TAG-LIGAND-[LINKER-]SUPPORT
or
SUPPORT-[LINKER-]LIGAND-[LINKER-]ENZYME
(collectively “iA”),
   wherein dashes represent bonds (covalent or non-covalent) and brackets represent optional elements.

In some embodiments, cold-active RNA polymerases (e.g., variant cold-active RNA polymerases) and compositions comprising one or more cold-active RNA polymerases (e.g., one or more variant cold-active RNA polymerases) may have any desirable form including, for example, a liquid, a gel, a film, a powder, a cake, and/or any dried or lyophilized form. A cold-active RNA polymerase composition may comprise a cold-active RNA polymerase (e.g., a wt or variant cold-active RNA polymerase) and a support or matrix, for example, a film, gel, fabric, column or bead comprising, for example, a magnetic material, agarose, polystyrene, polyacrylamide, and/or chitin. A cold-active RNA polymerase (e.g., a wt or variant cold-active RNA polymerase) and compositions comprising a cold-active RNA polymerase (e.g., a wt or variant cold-active RNA polymerase) may be active at lower temperatures. For example, a cold-active RNA polymerase (e.g., a wt or variant cold-active RNA polymerase) or a cold-active RNA polymerase composition (e.g., comprising a cold-active RNA polymerase or a variant cold-active RNA polymerase) may display RNA synthesis activity at 4° C.-35° C., 8° C.-32° C., 10° C.-37° C., 15° C.-35° C., 12° C.-32° C., 16° C.-32° C., 20° C.-32° C., 18° C.-30° C., or 20° C.-30° C. that exceeds the activity of T7 RNA polymerase (wild type) with the same template under the same conditions. Aqueous compositions (e.g., comprising a cold-active RNA polymerase or a variant cold-active RNA polymerase) may include, for example, one or more elements that reduce the composition's melting temperature including, for example, DMSO, methanol, glycerol, ethylene glycol, propylene glycol, sugars, amino acids, and proteins among others.

In some embodiments, a cold-active RNA polymerase (e.g., a wt or variant cold-active RNA polymerase) may be encoded by a nucleic acid sequence that, when transcribed, translated, and/or processed, results in an amino acid sequence having at least 75%, at least 80%, at least 85%, at least 88%, at least 90%, at least 91%, at least 93%, at least 95%, at least 96%, at least 97%, at least 98% or at least 99% identity to one or more of SEQ ID NOS: 1-29 (e.g., one of more of SEQ ID NOS: 1-19; one of more of SEQ ID NOS: 1-15). A nucleic acid encoding a variant cold-active RNA polymerase may be included in an expression cassette, expression vector, or other expressible form suitable for in vitro or in vivo expression (e.g., in E. coli or other bacteria or P. pastoris or other yeast). A nucleic acid encoding a cold-active RNA polymerase (e.g., a wt or variant cold-active RNA polymerase) may be modified or optimized (e.g., codon optimized) for expression in a desired organism or cell-free expression system.

### Methods and Workflows

The present disclosure further relates to methods and workflows that include a cold-active RNA polymerase (e.g., a wt or variant cold-active RNA polymerase). A method of producing a cold-active RNA polymerase may comprise, for example, contacting (a) a cold-active RNA polymerase transcript comprising an RNA encoding an amino acid sequence having (i) at least 75%, at least 80%, at least 85%, at least 88%, at least 90%, at least 91%, at least 93%, at least 95%, at least 96%, at least 97%, at least 98% or at least 99% identity to one or more of SEQ ID NOS: 1-29 (e.g., one of more of SEQ ID NOS: 1-19; one of more of SEQ ID NOS: 1-15), and (ii) optionally at least one conservative substitution relative to SEQ ID NO: 1 with (b) an expression system (e.g., a cell-based or cell-free expression system). A cold-active RNA polymerase transcript may be capped or uncapped, according to some embodiments. Uncapped RNA may be synthesized using solid-phase oligonucleotide synthesis chemistry or by transcribing a DNA (or RNA) template using a polymerase (e.g., a cold-active RNA polymerase) in an in vitro transcription reaction, for example. Capped RNA may be synthesized co-transcriptionally by contacting a template DNA encoding an RNA of interest with a cold-active RNA polymerase and a cap. In some embodiments, a composition may comprise a capping enzyme, S-adenosyl methionine (SAM), and/or a cap 2′0 methyltransferase enzyme (2′OMTase).

In some embodiments, a cold-active RNA polymerase (e.g., a wt or variant cold-active RNA polymerase) may be produced by contacting a cold-active RNA polymerase protein expression DNA construct operably linked to an expression control sequence (e.g., an appropriate promoter) to an in vitro transcription/translation system such as PURExpress In vitro Protein Synthesis Kit (New England Biolabs, Inc.) or TnT Quick Coupled Transcription/Translation System (Promega). In addition, a cold-active RNA polymerase (e.g., a wt or variant cold-active RNA polymerase) can be produced by contacting a cold-active RNA polymerase expression DNA construct under the control of an appropriate promoter to a cell-free protein synthesis system derived from organisms such as E. coli (e.g., NEBExpress Cell-free E. coli Protein Synthesis System (New England Biolabs, Inc.), rabbit, wheat germ, insect, or human. Reaction conditions (e.g., time, temperature, reaction composition) may be maintained or adjusted as needed to express the cold-active protein. Expressed cold-active protein may be purified by appropriate methods (e.g., chromatographic methods).

The present disclosure relates, in some embodiments, to methods for making an RNA of interest. An RNA of interest may be any RNA molecule including, for example, non-naturally occurring RNA, viral RNA, prokaryotic RNA, eukaryotic RNA, and/or archaeal RNA. An RNA of interest may be a messenger RNA (mRNA), ribosomal RNA (rRNA), transfer RNA (tRNA), small RNA (sRNA), microRNA (miRNA), long noncoding RNA (lncRNA), circular RNA (circRNA), aptamer RNA, antisense RNA, silencing RNA (siRNA), guide RNA (gRNA), or any combination thereof. An RNA of interest may be itself a therapeutic RNA or may be included in a therapeutic RNA composition. A method may comprise, for example, contacting a template DNA (or RNA) encoding the RNA of interest with a cold-active RNA polymerase (e.g., a wild type or a variant according to Table 1) to produce the RNA of interest. A template may comprise a cold-active promoter (e.g., a sequence having ≥70%, ≥75%, ≥80%, ≥85%, or ≥90% identity to SEQ ID NO:1) operably linked to the coding sequence for the RNA of interest. Contacting may include contacting at temperatures in ranges X to Y, where X is any of 1° C., 2° C., 3° C., 4° C., 5° C., 6° C., 7° C., 8° C., 10° C., 15° C., 20° C., 24° C., 25° C., 28° C. and Y is any of 5° C., 10° C., 15° C., 20° C., 24° C., 25° C., 28° C., 30° C., 32° C., 35° C. and X<Y. For example, contacting the polymerase and template may comprise contacting the two at a temperature in a range of 4° C.-35° C., 8° C.-32° C., 10° C.-37° C., 15° C.-35° C., 12° C.-32° C., 16° C.-32° C., 20° C.-32° C., 18° C.-30° C., or 20° C.-30° C. Contacting may further comprise suitable conditions for RNA synthesis including, for example, contacting the temple, polymerase, NTPs and optionally one or more modified NTPs. Contacting may further comprise contacting one or more of the foregoing in a composition comprising a buffer and/or having a pH in a range from X^{†} to Y^{†}, where X^{†} is any of pH 4, 4.5, 5, 5.5, 6, 6.5, 7, and Y^{†} is any of pH 6.5, 7, 7.5, 8, 8.5, 9, 9.5, 10, 10.5, 11 and X^{†}<Y^{†}. For example, a composition may have a pH from 6-9, 6.5-8.5 or 7-8. In some embodiments, a method may comprise contacting a cold-active RNA polymerase (e.g., a wt or variant cold-active RNA polymerase), a polynucleotide template (e.g., DNA or RNA) comprising a sequence encoding an RNA of interest (e.g., a therapeutic RNA), one or more NTPs, one or more modified NTPs, a buffer, and a salt (e.g., MgCl₂) at a temperature in a range of 4° C.-35° C., 8° C.-32° C., 10° C.-37° C., 15° C.-35° C., 12° C.-32° C., 16° C.-32° C., 20° C.-32° C., 18° C.-30° C., or 20° C.-30° C. and a pH in a range of 6-9, 6.5-8.5 or 7-8.

According to some embodiments, a method may further include capping the transcript, for example, by contacting the transcript and a capping enzyme (e.g., a Vaccinia capping enzyme, a Faustovirus capping enzyme). RNA transcript produced by a cold-active RNA polymerase (e.g., a wt or variant cold-active RNA polymerase) may comprise a smaller fraction of double-stranded RNA than similar RNA transcript produced by, for example, T7 RNA polymerase. For example, a cold-active RNA polymerase may produce RNA transcript having less than ½, less than ⅓, less than ¼, or less than 1/10 the double stranded RNA found in the same total quantity of RNA transcript produced by T7 RNA polymerase as measured, for example, by the CE and LC-MS methods of Examples 4 and 5 or by antibodies specific for dsRNA. Example methods may be found in U.S. Pat. No. 10,034,951. In some embodiments, RNA transcript produced by a cold-active RNA polymerase (e.g., a wt or variant cold-active RNA polymerase) may be less immunostimulatory than a like amount of a similar RNA transcript produced by, for example, T7 RNA polymerase. For example, a cold-active RNA polymerase may produce RNA transcript having less than ½, less than ⅓, less than ¼, or less than 1/10 the immunostimulatory activity of a like amount of a the same RNA transcript produced by T7 RNA polymerase as measured by, for example, interferon and/or cytokine expression by mammalian cells following exposure to such transcripts. RNA transcript produced by a cold-active RNA polymerase (e.g., a wt or variant cold-active RNA polymerase) may have increased homogeneity at the 3′ end of the transcript. For example, a cold-active RNA polymerase may produce an RNA transcript for which the majority of the RNA species have the same nucleotides at the 3′ end, and this proportion of RNA species with the same 3′ end may be ≥2X, ≥5X, ≥8X, ≥10X, ≥12X, ≥15X, ≥18X, ≥20X higher than a similar RNA transcript produced by, for example, T7 RNA polymerase. Example methods may be found in U.S. Pat. No. 10,034,951.

The present disclosure provides, in some embodiments, nucleic acid sequence based amplification (“NASBA”) methods using a cold-active RNA polymerase described herein. NASBA methods may be used as a rapid diagnostic test for pathogenic (among other) RNA viruses (e.g., influenza A, foot-and-mouth disease virus, severe acute respiratory syndrome (SARS)-associated coronavirus, HIV-1, human bocavirus (HBOV)) and also parasites like Trypanosoma brucei. NASBA methods are isothermal, often run at a constant temperature of at least 41° C. Use of a cold-active RNA polymerase (e.g., wildtype or a variant thereof) permits decreasing the reaction temperature (e.g., to 18° C.-32° C.). In some embodiments, an NASBA method comprises contacting a cold-active RNA polymerase, an RNA template, and a primer containing a promoter sequence wherein the primer hybridizes to a complementary site at the 3′ end of the template, and reverse transcriptase synthesizes the opposite, complementary DNA strand. RNAse H destroys the RNA template from the DNA-RNA hybrid, and a second primer hybridizes to the 5′ end of the cDNA strand. The second primer is extended using the cDNA as a template, resulting in double stranded DNA. An cold-active RNA polymerase may continuously produce complementary RNA strands of this template, which results in amplification. The amplicons are antisense to the original RNA template. A higher incubation temperature results in less non-specific binding of DNA primers to the RNA. In some embodiments, the reaction may include a temperature-sensitive inhibitor of the polymerase, thereby allowing the polymerase to remain inactive until the temperature rises.

The present disclosure provides, in some embodiments, transcription-mediated amplification (“TMA”) methods using a cold-active RNA polymerase described herein. TMA methods may be performed as isothermal, single-tube nucleic acid amplifications using two enzymes, a cold-active RNA polymerase and reverse transcriptase, to rapidly amplify a target RNA/DNA. TMA may be configured to provide simultaneous detection of multiple pathogenic organisms in a single tube, allowing, for example, clinical laboratories to perform nucleic acid test (NAT) assays for blood screening with fewer steps, less processing time, and faster results. It may be used in molecular biology, forensics, and medicine for the rapid identification and diagnosis of pathogenic organisms. In contrast to similar techniques such as polymerase chain reaction and ligase chain reaction, this method involves RNA transcription (via an RNA polymerase) and DNA synthesis (via reverse transcriptase) to produce an RNA amplicon (the source or product of amplification) from a target nucleic acid. This technique can be used for both target RNA and DNA.

A method of making an RNA of interest may further comprise contacting the produced RNA with a one or more pharmaceutically acceptable additives (e.g., excipients, diluents, and/or carriers), including, for example, fluids, solvents, dispersion media, wetting agents, crowding agents, micelles, lipidoids, liposomes, polymers, lipoplexes, peptides, proteins, salts, surface active agents, isotonic agents, thickeners, emulsifiers, preservatives, stabilizers, solubilizers, buffers, sugars, starches, cellulose, waxes, glycols, polyols, polyesters, polycarbonates, polyanhydrides, hyaluronidase, nanoparticles (e.g., lipid nanoparticles, core-shell nanoparticles, and/or nanoparticle mimics), and combinations thereof. In some embodiments, pharmaceutically acceptable additives protect, preserve, and/or stabilize an RNA of interest during manufacture, storage, use, and/or administration to a subject. Examples of pharmaceutical acceptable additives include those described in U.S. Patent Publication No. 2017/0119740. A method of making an RNA of interest may further comprise contacting the RNA with one or more additives selected from lipidoids, liposomes, polymers, lipoplexes, peptides, proteins, cells transfected with HCMV RNA vaccines (e.g., for transplantation into a subject), hyaluronidase, nanoparticles (e.g., lipid nanoparticles, core-shell nanoparticles, and/or nanoparticle mimics).

Manufactured RNAs may be formulated for delivery and/or delivered to a eukaryotic organism. Examples of subjects that may receive a manufactured RNA include humans and non-human animals (e.g., mouse, rat, rabbit, dog, cat, cattle, swine, sheep, horse or primate). Manufactured RNAs may be delivered to plants or plant cells, according to some embodiments, to confer or augment resistance to or tolerance of an environmental condition (e.g., drought, salt) and/or to prevent, mitigate or treat herbivory, pathogen infection, or the effects thereof. Manufactured RNA also may be delivered to one or more yeast cells.

In some embodiments, the present disclosure provides methods for preparing an RNA dosage form comprising, contacting a cold-active RNA polymerase and a transcript encoding the RNA of interest to produce a transcribed RNA, optionally capping the transcribed RNA with a capping enzyme to form a capped RNA, and contacting produced RNA or the capped RNA with one or more pharmaceutically acceptable additives, binders, buffers, coatings, colors, controlled release agents, delivery agents (e.g., liposomes, propellants), diluents, disintegrants, dyes, excipients, fillers, lipids, lubricants, salts, sorbants, stabilizers, and/or other agents to produce an RNA dosage form. An RNA of interest may be combined with (e.g., in a single dosage form) or delivered concurrently or in sequence with one or more other active pharmaceutical agents. An RNA and/or its encoded translation product(s) may function in a subject as an active pharmaceutical agent, according to some embodiments. An RNA (e.g., a capped RNA dosage form) may be administered by any suitable route of administration, including transdermal, oral, enteral, parenteral, ocular, ottic, transmucosal, sublingual, and pulmonary (e.g., by nebulization and/or inhalation) routes, and combinations thereof.

An RNA of interest can either be naked or formulated in a suitable form for delivery to a subject, e.g., a human. Formulations can include liquid formulations (solutions, suspensions, dispersions), topical formulations (gels, ointments, drops, creams), liposomal formulations (such as those described in: U.S. Pat. No. 9,629,804 B2; US 2012/0251618 A1; WO 2014/152211; US 2016/0038432 A1). The cells into which the RNA product is introduced may be in vitro (i.e., cells that have been cultured in vitro on a synthetic medium). Accordingly, the RNA product may be transfected into the cells. The cells into which the RNA product is introduced may be in vivo (cells that are part of a mammal). The cells into which the RNA product is introduced may be present ex vivo (cells that are part of a tissue, e.g., a soft tissue that has been removed from a mammal or isolated from the blood of a mammal).

### Kits

The present disclosure further relates to kits including a cold-active RNA polymerase (e.g., a wt or variant cold-active RNA polymerase). For example, a kit may include a cold-active RNA polymerase (e.g., a wt or variant cold-active RNA polymerase) and NTPs, other enzymes (e.g., other polymerases, capping enzymes, others), buffering agents, or combinations thereof. Enzymes may be included in a storage buffer. Any suitable storage buffer may be used, for example, buffers comprising one or more of a cryoprotectant (e.g., a polyol such as glycerol, an antifreeze protein), a salt, a detergent, a reducing agent, a sugar, a chelator, and an antimicrobial agent and having a pH tolerated by the enzyme to be stored, for example, between pH 6 and 9. A composition or kit may include a reaction buffer which may be in concentrated form, and the buffer may contain additives (e.g. glycerol), salt (e.g. NaCl, KCl), reducing agent, EDTA or detergents, among others. Detergents include nonionic detergents (e.g., t-octylphenoxypolyethoxyethanol), anionic detergents (e.g., alkylbenzene sulfonates), cationic detergents (e.g., alkylbenzene quaternary ammonium), and zwitterionic detergents. A composition or kit comprising rNTPs may include one, two, three of all four of rATP, rUTP, rGTP and rCTP. A kit may further comprise one or more modified nucleotides. A kit may optionally comprise one or more primers (random primers, bump primers, exonuclease-resistant primers, chemically-modified primers, custom sequence primers, or combinations thereof).

A kit may be a non-natural collection of components configured, for example, for convenient storage, shipping, delivery, and/or use. One or more components of a kit may be included in one container for a single step reaction, or one or more components may be contained in one container, but separated from other components for sequential use or parallel use. The contents of a kit may be formulated for use in a desired method or process.

A kit is provided that contains: (i) a cold-active RNA polymerase (e.g., a wt or variant cold-active RNA polymerase); and (ii) a buffer. A cold-active RNA polymerase (e.g., a wt or variant cold-active RNA polymerase) may have a lyophilized form or may be included in a buffer (e.g., a storage buffer or a reaction buffer in concentrated form). A kit may contain a cold-active RNA polymerase (e.g., a wt or variant cold-active RNA polymerase) in a mastermix suitable for receiving and amplifying a template nucleic acid. A cold-active RNA polymerase (e.g., a wt or variant cold-active RNA polymerase) may be a purified enzyme so as to contain substantially no DNA or RNA and no nucleases. The reaction buffer in (ii) and/or storage buffers containing the RNA polymerase in (i) may include a non-ionic surfactant, an ionic surfactant (e.g. an anionic or zwitterionic surfactant) and/or a crowding agent. A kit may include a cold-active RNA polymerase (e.g., a wt or variant cold-active RNA polymerase) and the reaction buffer in a single tube or in different tubes.

A subject kit may further include instructions for using the components of the kit to practice a desired method. The instructions may be recorded on a suitable recording medium. For example, instructions may be printed on a substrate, such as paper or plastic, etc. As such, the instructions may be present in the kits as a package insert, in the labeling of the container of the kit or components thereof (i.e., associated with the packaging or subpackaging) etc. Instructions may be present as an electronic storage data file residing on a suitable computer readable storage medium (e.g. a CD-ROM, a flash drive). Instructions may be provided remotely using, for example, cloud or internet resources with a link or other access instructions provided in or with a kit.

### EXAMPLES

Some specific example embodiments may be illustrated by one or more of the examples provided herein.

### EXAMPLE 1: Materials

Plasmids for cold-active RNA polymerase overexpression and for use as templates in IVT reactions were ordered from GenScript (Piscataway, NJ, USA) or prepared by site-directed mutagenesis and are represented by SEQ ID NOS: 47, 48, 51, 53, 54, 56, 57, 59, 60, 62, 63, 65, 66, 91, 92. All plasmid sequences were verified by Oxford Nanopore Sequencing using the Rapid Barcoding Kit (SQK-RBK114.96, ONT, Oxford, UK) and the clone validation assembly workflow. Linearized plasmids were prepared by digestion with BspQI (NEB) and assessed by agarose gel electrophoresis. Oligonucleotides were purchased from IDT (Coralville, IA, USA). Trinucleotide cap analogs were purchased from Northern RNA (Calgary, CN) and modified nucleotides were purchased from Trilink (San Diego, CA, USA). Unless otherwise noted, all enzymes and reagents were provided by New England Biolabs (NEB).

### EXAMPLE 2: Cold-Active RNA Polymerase Promoter Identification

Genomes of bacteriophages given the taxonomic assignment of Colwellvirinae and Molineauxvirinae were collected from the RefSeq database using the webserver search function (Nucleic Acids Res. 44, D733-D745 (2016); Microb Biotechnol 13, 1428-1445 (2019)). A phylogenetic tree was built from whole genomes with VICTOR using the do formula and visualized with the ETE 3 toolkit (Bioinformatics 33, 3396-3404 (2017); Mol. Biol. Evol. 33, 1635-1638 (2016)). To identify transcriptional promoters a BLAST alignment was performed using the cold-active RNA polymerase genomes as both the query and subject sequence with a cutoff E-value of 0.1 and high scoring pairs were inspected (J. Mol. Biol. 215, 403-410 (1990)). Promoters identified by BLAST analysis were used to construct a position-specific weight matrix and search the genome for additional instances of the motif with PWMScan (Bioinformatics 34, 2483-2484 (2018)).

### EXAMPLE 3: Recombinant Purification of Cold-Active RNA Polymerase

An expression vector for cold-active RNA polymerase with an N-terminal hexahistidine tag was designed using the pET28 vector. Competent E. coli cells (C3013, NEB) were transformed with the expression plasmid and grown at 37 C with 220 RPM shaking to an optical density of 0.6 absorbance units. The temperature was reduced to 16° C., IPTG was added to a final concentration of 0.5 mM and protein overexpression occurred during overnight incubation of the culture. Cultures were centrifuged (5000 rcf, 4 C, 30 min) and cell pellets were resuspended in Buffer A (50 mM Tris-HCl pH 8.0, 300 mM NaCl, 20 mM imidazole, 10% v/v glycerol) and lysed by sonication. Cellular debris was pelleted by centrifugation (15000 rcf, 4 C, 45 min) and the clarified lysate was applied to a HisTrap FF column (Cytiva, Marlborough, MA, USA). Bound protein was eluted with a step gradient to Buffer B (50 mM Tris-HCl pH 8.0, 0.1 M NaCl, 0.3 M imidazole, 10% v/v glycerol) and fractions were analyzed by SDS-PAGE (ThermoFisher, Waltham, MA, USA). Fractions containing cold-active RNA polymerase were pooled, diluted in Buffer B and applied to a HiTrap Heparin HP column (Cytiva). Protein was eluted with a linear gradient to Buffer C (50 mM Tris-HCl pH 8.0, 1.0 M NaCl, 1 mM DTT, 1 mM EDTA, 10% v/v glycerol), then diluted in Buffer D (50 mM Tris-HCl pH 8.0, 0.05 M NaCl, 1 mM DTT, 1 mM EDTA, 10% v/v glycerol), applied to a HiTrap Q FF column (Cytiva), and finally eluted with a linear gradient to Buffer C. Fractions containing cold-active RNA polymerase were pooled and dialyzed against storage buffer (50 mM Tris-HCl pH 8.0, 100 mM NaCl, 0.5 mM TCEP, 0.1 mM EDTA, 50% v/v glycerol). Final purity of cold-active RNA polymerase was judged to be ≥95% by SDS-PAGE and the concentration was measured with a Bradford assay. Purified protein was stored at −20 C until use.

### EXAMPLE 4: In Vitro Transcription Reactions

Transcription reactions (25 μL) were performed in IVT buffer (40 mM Tris-HCl pH 7.9, 20 mM MgCl2, 1 mM DTT, 2 mM spermidine) containing 5 mM of each NTP or modified nucleotide (m⁶A, 5moU, 5hmU, 5mC, ψ, or m¹ψ), 20 U murine RNAse inhibitor (NEB), 0.05 U E. coli inorganic pyrophosphatase (NEB), and 1 μg of linear DNA template. Reactions were pre-incubated at the appropriate temperature for 10 minutes, and transcription was initiated by addition of either T7 or cold-active RNA polymerase. Unless otherwise indicated, IVT was performed with cold-active RNA polymerase at 25 C and with T7 at 37 C. After incubation for 1 hour, reactions were treated with TURBO DNase (ThermoFisher) at 37 C for 30 minutes and the RNA was purified using a Monarch RNA Cleanup Kit (T2050, NEB), or the reaction was quenched with 2x buffer (20 mM Tris-HCl pH 8.0, 100 mM EDTA, 0.2% Triton X-100) and analyzed immediately. For determination of RNA yield, 5 μL of serially diluted quenched IVT reaction was combined with 95 μL Qubit RNA BR buffer (ThermoFisher) and fluorescence (Ex: 625 nm; Em: 655 nm) was measured using a SpectraMax ID5 (Molecular Devices, San Jose, CA, USA) plate reader. Agarose gel electrophoresis was performed with RNA samples denatured by heating to 70 C for 5 minutes in RNA Loading Dye (NEB) and gels were stained with SYBR Gold (ThermoFisher) and visualized on a Typhoon scanner (Cytiva).

### EXAMPLE 5: Cold-Active RNA Polymerase Promoter Optimization

Polymerase chain reaction (PCR) primers were designed to include the 19 nt Njord transcriptional promoter (SEQ ID NO:33) or all 57 possible single-nucleotide substitutions. For example, the thymidine nucleotide at position-17 in the promoter was replaced by each of the other three nucleotides. Saturation mutagenesis in this way for 19 positions in the promoter sequence yield 57 total sequences under investigation. PCR was carried out using Q5 High Fidelity 2x Master Mix (M0492, NEB) using a pUC-derived plasmid as template to produce SEQ ID NO:51 with all 57 possible single-nucleotide substitutions. PCR amplicons were purified using magnetic beads (NEB) and a KingFisher Flex (ThermoFisher), assessed with the TapeStation High Sensitivity D5000 kit (Agilent) and quantified with a Nanodrop 8000 (Nanodrop). IVT reactions were performed in triplicate using 250 ng of template at 25 C for 60 minutes followed by quenching of the reaction, serial dilution and RNA quantification.

### EXAMPLE 6: Cold-Active RNA Polymerase Transcriptional Fidelity

An assay developed for polymerase fidelity measurement with a Pacific Biosciences sequencing platform was applied to Njord and T7 RNA polymerase at 25 C. Standard in vitro transcription reactions were performed with 300 ng of a linearized plasmid template designed to include all trinucleotide sequences (SEQ ID NOS: 53, 54, 56, 57), except that T7 RNA polymerase reactions were performed with 10 mM NTPs and incubated for 2 hours. Synthesis of cDNA, Pacific Biosciences SMRTBell library preparation, sequencing on a Sequel system, and data analysis were performed as described previously (Sci. Rep. 12, 13017 (2022)).

### EXAMPLE 7: Analysis of Cap Analog Incorporation

Co-transcriptional capping IVT reactions (20 μL) were performed in IVT buffer with 5 mM each NTP, 5 mM trinucleotide cap analog, and 1 μg linearized plasmid (SEQ ID NOS: 59, 60) with promoters and initiating nucleotides corresponding to the cap analog (SEQ ID NOS: 43-46 and 71-74). Concentrations of NTPs were varied when anti-reverse cap analog (ARCA, NEB) was used. RNA was purified using a Monarch RNA Cleanup Kit and the concentration was measured with a Nanodrop 8000 (Nanodrop). Incorporation of cap analogs was measured as previously described (ACS Pharmacol. Transl. Sci. 6, 1692-1702 (2023)). Purified RNA (15 fmole) was combined with a complementary biotinylated DNA oligo (30 fmole), heated to 80° C. for 2 minutes then gradually cooled to 20 C. NEBuffer r1.1 was added to a final 1x concentration (10 mM Bis-Tris-Propane-HCl pH 7, 10 mM MgCl2, 100 μg/mL recombinant BSA) and human RNase IV (0.1 ng, NEB) was used to digest the RNA at 37 C for 1 hour and then inactivated by addition of 50 U murine RNase inhibitor (NEB). The RNA/DNA heteroduplex was purified with streptavidin beads (NEB) and eluted by heating the beads in water to 80° C. for 5 minutes.

### EXAMPLE 8: Assays for dsRNA Formation

To assay DNA-terminus initiated transcription, IVT templates designed to produce unstructured RNA were generated by PCR (SEQ ID NOS: 65, 66). Purified PCR amplicons (1 μg) were used as input for IVT at 30 C, DNA was digested and the RNA was purified. RNA was subjected to digestion by either RNAse III in the presence of manganese (M0245, NEB) or by RNAse If (M0243, NEB) and run on a 6% polyacrylamide TBE Gel (EC6265BOX, ThermoFisher). Gels were stained with either SYBR Gold or 4.3 μM acridine orange (A1301, ThermoFisher) and visualized on a Typhoon scanner.

A homogeneous time resolved fluorescence (HTRF) dsRNA-detection kit (64RNA PEG, Revvity, Waltham, MA, USA) was used to measure dsRNA content in IVT RNA (Anal. Biochem. 566, 46-49 (2019)). Quenched IVT reactions were serially diluted into 1x quench buffer in a 96-well plate alongside the Qubit ssRNA standard and the HTRF dsRNA standard. For measurement of total RNA, samples were diluted 20-fold in Qubit BR RNA detection buffer and fluorescence was measured using a SpectraMax ID5 plate reader. For measurement of dsRNA, samples were diluted 10-fold into the kit lysis buffer, and then 10 μL was combined with 10 μL of a 1:1 donor: acceptor antibody mix. HTRF was measured using a SpectraMax ID5 plate reader equipped with a TRF Enhancement Module according to the manufacturer's instructions.

### EXAMPLE 9: Identification and Initial Characterization of Cold-Active RNA Polymerase

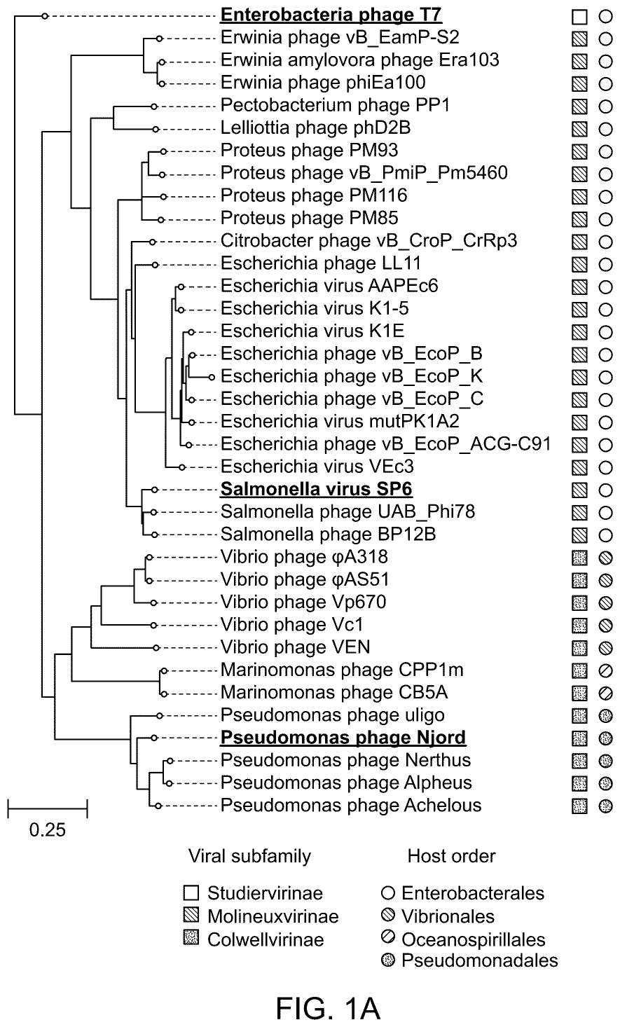

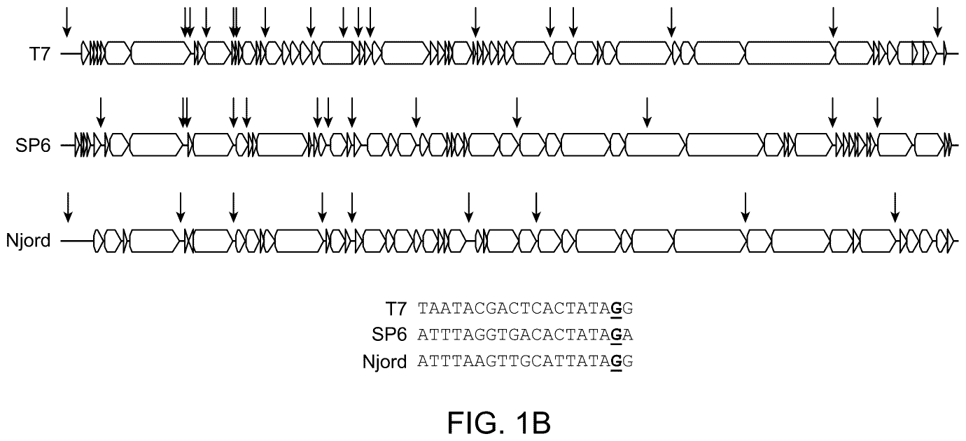

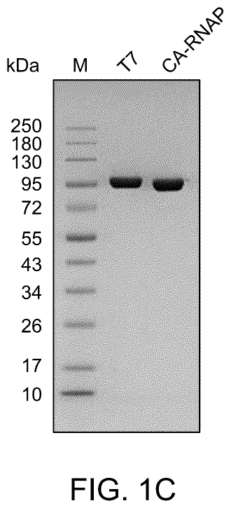

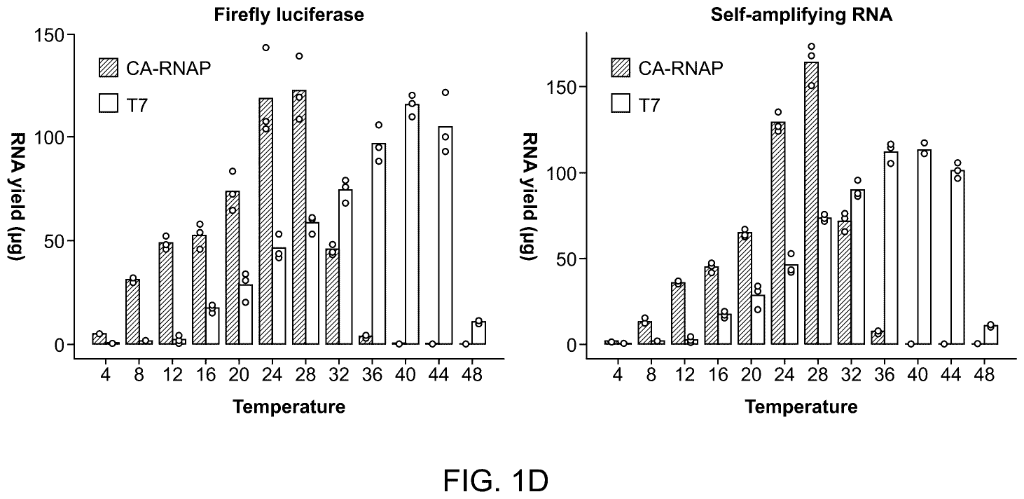

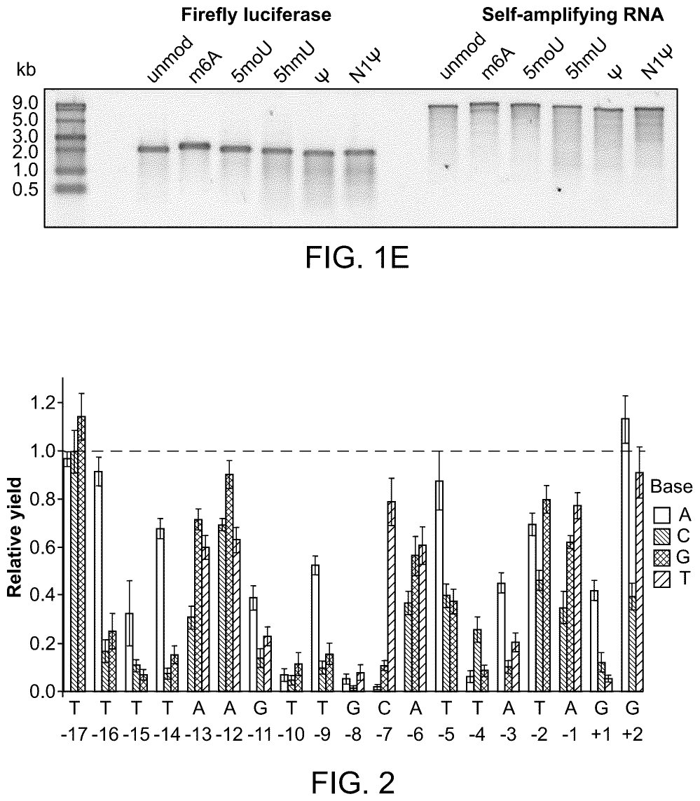

Salmonella phage SP6 belongs to the Molineuxvirinae, a viral subfamily in the taxonomy established by the International Committee on Taxonomy of Viruses. Bacteriophages in this subfamily infect different species of Enterobacteriacae, while those in the related subfamily Colwellvirinae target hosts in the genera Vibrio, Pseudomonas and Marinomonas (FIG. 1A). Isolates of Colwellvirinae have been obtained from wastewater, aquaculture tanks and the Mediterranean Sea. The Vibrio phage strains qA318 and qAS51 were isolated from aquaculture waterways and efficiently form plaques when grown at 25° C. (BMC Genomics 15, 505 (2014)). Colwellvirinae may then be expected to thrive, e.g. carry out rapid lytic infection of the host, in cooler environments than the Molineuxvirinae. Given the central role of RNA polymerase in the bacteriophage lytic cycle, we reasoned that Colwellvirinae RNA polymerases may be adapted for efficient transcription at low temperatures. One cold-active RNA polymerase was chosen as a representative Colwellvirinae RNA polymerase and tested for its temperature dependence and utility for in vitro mRNA synthesis.

IVT templates may comprise a coding sequence of interest and a promoter operably linked to the coding sequence. Each RNA polymerase may recognize a different promoter or set of promoters than other RNA polymerases and IVT reactions with mismatched RNA polymerases and promoters may result in little or no transcription product. The following steps were taken to identify cognate promoter sequence(s) for cold-active RNA polymerase and may be adapted to identify cognate promoter sequences for other cold-active RNA polymerases.

Searching the Njord phage genome for repeated sequences revealed a motif dispersed throughout intergenic regions, which has homology to the transcriptional promoter of Salmonella phage SP6. These repeated sequences were assigned as promoters and used to build a position-specific weight matrix and rescan the genome, resulting in identification of nine candidate transcriptional promoters in Pseudomonas phage Njord (FIG. 1B and SEQ ID NOS: 31-41). T7, SP6 and cold-active RNA promoters share common elements that include an AT-rich 5′ sequence, a 3′ TATA sequence, and a guanosine residue incorporated as the first nucleotide in the transcript.

Two linearized plasmids containing the appropriate promoter sequences were used as templates for IVT reactions (SEQ ID NOS: 47, 48, 59, 60). The encoded transcripts represent a standard mRNA and a self-amplifying mRNA and are 2.1 and 9.7 kb in length, respectively. Each has a different 5′ untranslated region, but both start with guanosine and terminate in a polyA sequence. In vitro transcription was performed with these templates and T7 or cold-active RNA polymerase (FIG. 1C) at different temperatures. Apparent RNA yields of cold-active RNA and T7 RNA polymerases show similar temperature profiles with both enzymes retaining activity below their optima and showing a precipitous drop in RNA yield at elevated temperatures (FIG. 1D). The temperature optimum for transcription by cold-active RNA polymerase is 10 to 15° C. lower than that of T7 RNA polymerase, but both enzymes have comparable RNA yields at their respective optima. Using the saRNA template, cold-active RNA polymerase approaches the theoretical limit of RNA synthesized in a 25 μL reaction using 20 mM total NTPs at 28° C., and at 12° C. produces tens of micrograms of RNA. The high yields of cold-active RNA polymerase at reduced temperature and the lack of activity above 30° C. indicate that this enzyme is adapted to cooler environments than T7 and SP6 polymerases.

### EXAMPLE 10: Synthesis of RNA with Modified Nucleotides Using Cold-Active RNA Polymerase

Therapeutic mRNAs may be synthesized with modified nucleotides to mitigate undesirable immune stimulation following administration of the therapeutic mRNA. The ability of a cold-active RNA polymerase to incorporate the modified nucleotides N6-methyladenosine (m⁶A), 5-hydroxymethyluridine (5hmU), 5-methoxyuridine (5moU), pseudouridine (Y′), and N1-methylpseudouridine (N1m′P′) into RNA was tested. Production of full-length 2.1 kb and 9.7 kb transcripts indicate complete incorporation of these modified nucleotides (FIG. 1E). Incomplete transcripts are apparent under the conditions used, and their formation appears to be dependent on the nucleotides supplied in the IVT reaction. Nevertheless, cold-active RNA polymerase is clearly capable of producing full-length transcripts up to about 10 kb in length with a variety of nucleotide modifications.

### EXAMPLE 11: Impact of Promoter Selection on RNA Yield Using Cold-Active RNA Polymerase

Selection of the bacteriophage promoter in the IVT template influences the transcriptional RNA yield. The T7 10 promoter is commonly used with T7 RNA polymerase owing to its high promoter strength. Initial transcription reactions with cold-active RNA polymerase used a promoter located upstream of the structural genes in analogy to T7 φ10. To identify the optimal Njord promoter and gain insight into promoter sequence specificity, every position in the Njord promoter was systematically replaced with each other nucleotide. For the 19 positions considered, which include the first two initiating nucleotides (+1 and +2), this corresponds to 58 promoter sequences for which transcriptional yields were measured (FIG. 2). A preference for guanosine at the +1 position is observed although adenosine may also be used as the initiating nucleotide with lower yield. Most nucleotide replacements reduce the transcriptional yield. Mutation of nucleotides at positions −8 and −10 strongly attenuates transcription, indicating a polymerase-promoter binding mode in common with T7 RNA polymerase. Two replacements, T to G at position −17 and G to A at position +2, marginally increase RNA yield relative to the reference promoter. The resulting sequence corresponds to one of the identified Njord promoters (SEQ ID NO:17), located downstream of putative metabolic genes and directly upstream of three predicted open reading frames encoding proteins lacking known function but which are conserved among the Colwellvirinae. In this light, using a promoter sequence comprising GTTTAAGTTGCATTATAGA (SEQ ID NO: 32) with a cold-active RNA polymerase may maximize RNA yield under like circumstances.

### EXAMPLE 12: Transcriptional Fidelity and Co-Transcriptional Capping with Cold-Active RNA Polymerase

Robust RNA synthesis at low temperature is a compelling feature of cold-active RNA polymerase and prompted a more detailed investigation into the enzyme, with attention to properties relevant for therapeutic mRNA synthesis. Transcriptional fidelity describes the selectivity of the RNA polymerase for the correctly base-paired nucleotide during transcription. A low-fidelity polymerase will produce transcripts encoding mutated proteins, with potentially dangerous consequences. We used an established Pacific Biosciences Single Molecule Real-Time (SMRT) sequencing-based assay to measure the error rates of first-strand cDNA synthesis (Sci. Rep. 12, 13017 (2022); Nucleic Acids Res. 46, 5753-5763 (2018)). This apparent error rate represents the combined errors of RNA polymerase and reverse transcriptase (RT) but allows for comparison between polymerases when the same RT is used.

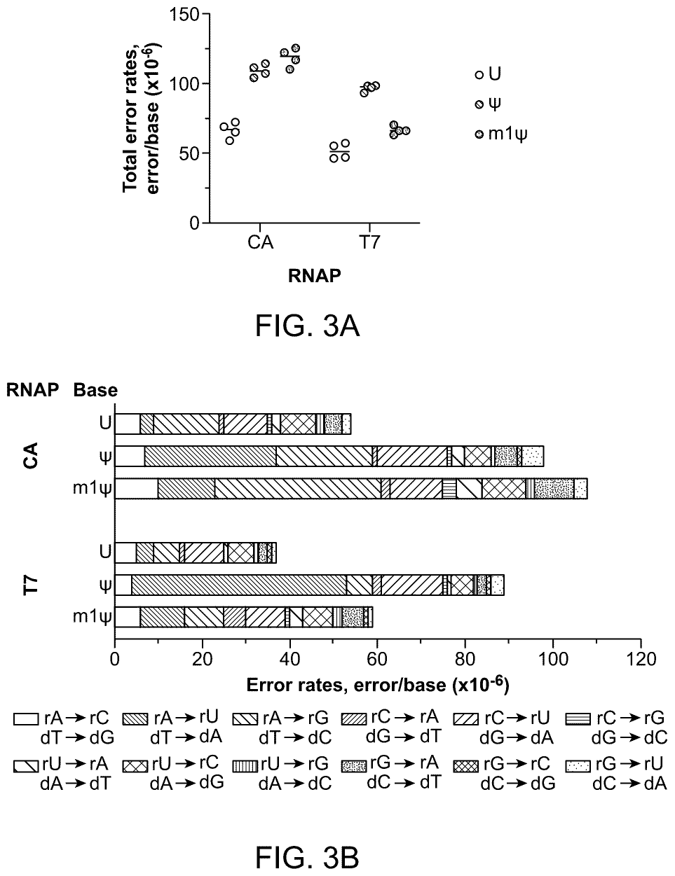

Cold-active RNA polymerase in vitro transcribed unmodified RNAs with a combined error rate of 67+8×10⁻⁶ errors/base (FIG. 3A). For the ψ and m1ψ incorporation, the combined error rates were 105+6×10⁻⁶ and 118+6×10⁻⁶ errors/base, respectively, about 1.6-fold and 1.8-fold compared to unmodified uridine incorporation. The combined error rates of T7 RNA polymerase for unmodified and w-incorporated RNA were 51±6×10⁻⁶ and 97±3×10⁻⁶ errors/base, respectively, 0.8-fold compared to those of cold-active RNA polymerase. Noticeably, the combined error rate of T7 RNA polymerase for mlw-incorporated IVT was 66±3×10⁻⁶ errors/base, about 0.6-fold compared to that of cold-active RNA polymerase. To further understand the errors when modified uridines was incorporated by cold-active RNA polymerase, we characterized the combined errors for transcripts made with all the three uridine analogs. The predominant error type was single base substitution ranging from 80% to 92%, which is comparable to T7 RNA polymerase ranging from 74% to 91%. The highest base substitution in unmodified RNA transcribed with cold-active RNA polymerase was rA-to-rG/dT-to-dC (15×10⁻⁶ errors/base) (FIG. 3B). For the modified RNA, the highest substitute error in w-incorporated RNA became rA-to-rU/dT-to-dA (30×10⁻⁶ errors/base), while the highest in mlw-incorporated transcript remained to be rA-to-rC/dT-to-dG (38×10⁻⁶ errors/base). In contrast, the highest base substitution in unmodified RNA transcribed with T7 RNA polymerase was rC-to-rU/dG-to-dT (9×10⁻⁶ errors/base). In both w- and mly-incorporated RNA, the highest substitution error was rA-to-rU/dT-to-dA (49×10⁻⁶ and 10 x 10⁻⁶ errors/base, respectively). SP6 RNA polymerase discriminates uridine and mly more effectively than T7 RNA polymerase (Nucleic Acids Res. 51, 1914-1926 (2023)), a result that may be related to the reduced fidelity of incorporation of mly by cold-active RNA polymerase.

Having confirmed that the fidelity of cold-active RNA polymerase is comparable to T7 RNA polymerase, the ability of the RNA polymerase to produced capped mRNA was assessed. A typical capped mRNA contains an N7-methylguanosine (m⁷G) residue attached to the first nucleotide of the transcript with a 5′-5′ triphosphate linkage. Installation of this covalent modification in therapeutic mRNAs can be accomplished by supplying the IVT reaction with short oligonucleotides which T7 RNA polymerase can use to initiate transcription. When the dinucleotide anti-reverse cap analog (ARCA, (m₂^{7,3′-O}G) ppp (G)) is used in molar excess of GTP in IVT reactions, the resulting mRNA may be over 70% capped. This approach was extended using trinucleotide cap analogs, including (m⁷G) ppp (2′-OMeA) pG (Cap AG), which produce mRNA in high yield that is ≥90% capped.

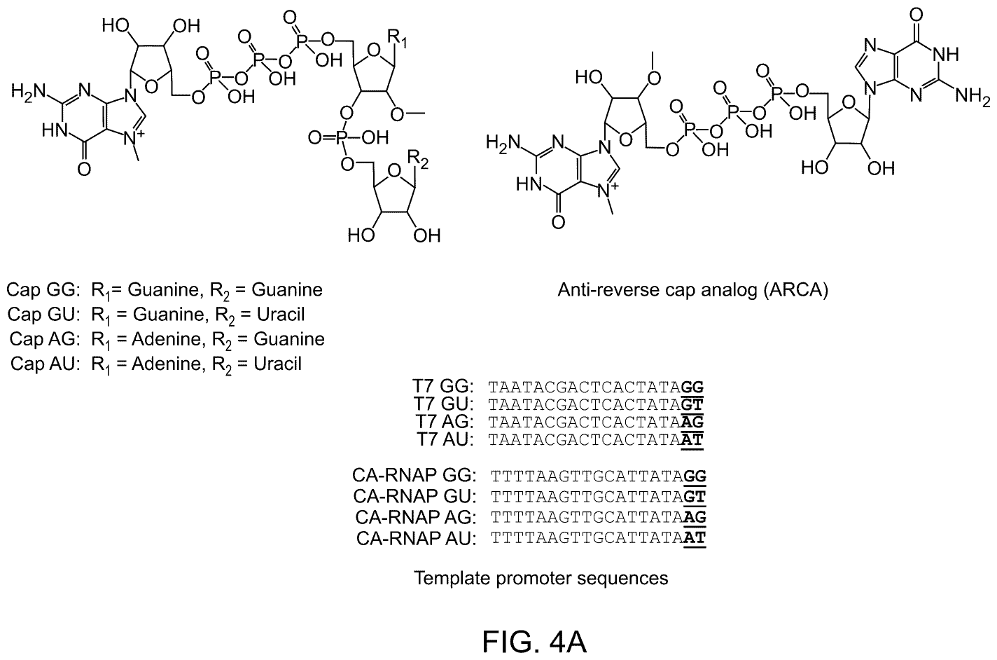

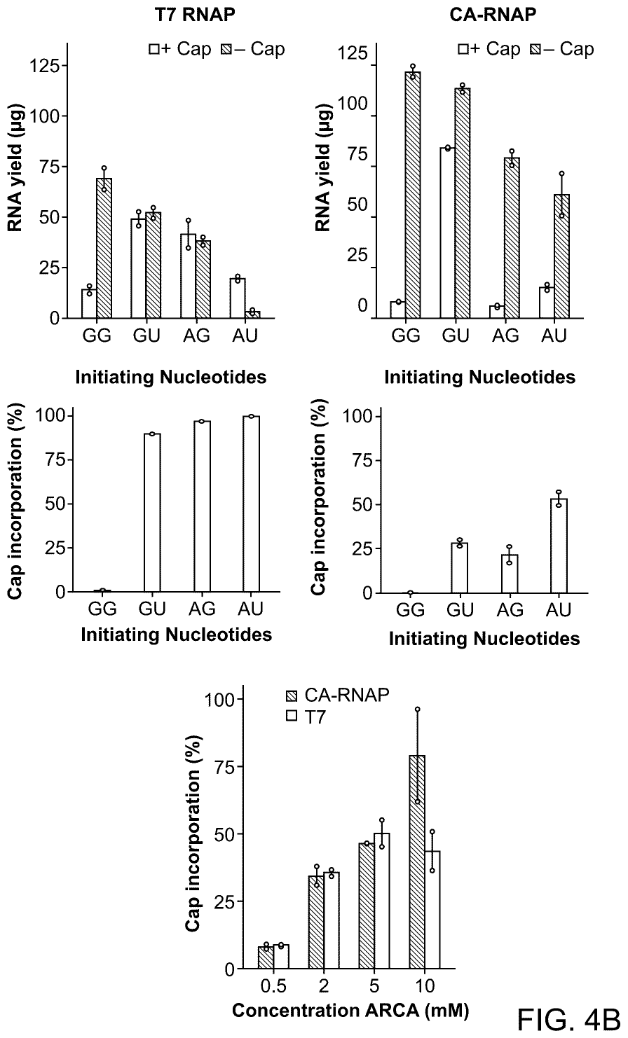

Cold-active RNA polymerase and T7 RNA polymerase were tested for their abilities to incorporate ARCA and a panel of commercially available trinucleotide cap analogs. Trinucleotide caps all contained a m⁷G linked by a 5′-5′ triphosphate to a 2′OMe modified dinucleotide. The nucleobases of the dinucleotide portion were varied to match the +1 and +2 positions of an appropriate IVT template (FIG. 4A). Transcriptional yields were measured using these templates with and without addition of cap analogs (FIG. 4B). Inclusion of trinucleotide caps at equimolar concentration with all four NTPs tended to reduce RNA yield, except for Cap-AU which rescued the activity of T7 RNA polymerase on the T7-AU template. Strikingly and in contrast to T7 RNA polymerase, Cap-AG appeared to be a competitive transcriptional inhibitor of cold-active RNA polymerase when the AG promoter (SEQ ID NO: 45) was used.

Mass spectrometry was used to assess the 5′ mRNA structure of these IVT products (FIG. 4B). An assay was employed which uses a biotinylated DNA oligo to protect the 5′ mRNA end from digestion by human RNase 4 (ACS Pharmacol. Transl. Sci. 6, 1692-1702 (2023)). When only NTPs are included in IVT, the mass of the 5′ mRNA fragment corresponds to the expected sequence with a 5′ppp or 5′pp group. Neither polymerase produces significant amounts of transcripts with inhomogeneous 5′ ends using these promoters and initiating sequences. T7 RNA polymerase efficiently incorporates all of the cap analogs except Cap-GG and in the case of Cap-AU the proportion of capped oligo approaches 100%. This polymerase prefers to initiate from these trinucleotides when NTPs are available, whereas cold-active RNA polymerase displays the opposite behavior. Regardless of the dinucleotide combination used in this example, cold-active RNA polymerase did not incorporate the trinucleotide cap analog with the high efficiency of T7 RNA polymerase under the conditions tested. This is not the case for ARCA, which both polymerases discriminate against GTP to similar degrees (FIG. 4B). When ARCA is used in 5-fold molar excess over GTP, cold-active RNA polymerase is capable of co-transcriptional capping to ˜80% efficiency, demonstrating that the polymerase is not specifically discriminating against the pre-installed m⁷G 5′ cap. Advances made in co-transcriptional capping with T7 RNA polymerase evidently do not transfer to cold-active RNA polymerase, though there may be other cap analog oligonucleotides which function well with cold-active polymerases. These include trinucleotide cap analogs with tetraphosphate linkages, disrupted ribose rings, additional covalent modifications, longer oligonucleotides with more capping analogs, and pairing of existing trinucleotide analogs with DNA templates of different sequences.

### EXAMPLE 13: Analysis of Cold-Active RNA Polymerase Reaction Products

T7 RNA polymerase produces varied RNA species in addition to the desired run-off transcript, including short ‘abortive’ transcripts, transcripts with heterogenous 5′ ends, transcripts with extended 3′ ends, and even full-length antisense transcripts. Overextension of the 3′ end may occur in a template-independent manner, or in a templated mechanism wherein the 3′ RNA end folds on itself and primes T7 RNA polymerase for RNA-dependent RNA polymerase (RdRp) activity. This self-templated mechanism can lead to the production of immunostimulatory dsRNA species. dsRNA species are also formed via DNA-terminus initiated transcription, in which T7 RNA polymerase synthesizes an RNA fully complementary to the desired transcript and produces a long dsRNA molecule. Not all T7 homologs display these aberrant behaviors.

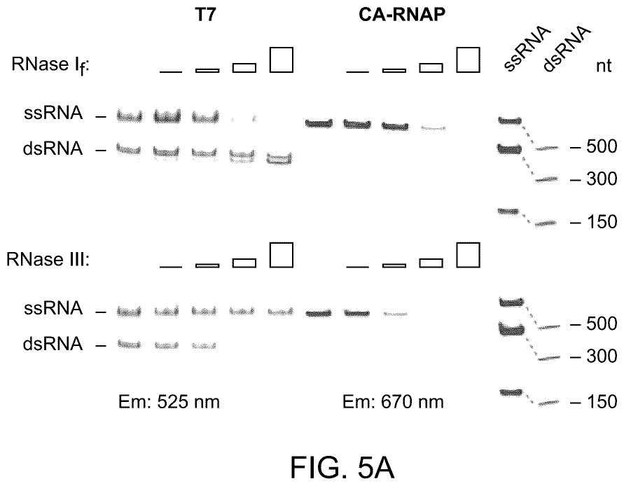

One experiment using a model substrate was used to observe DNA-terminus initiated transcription activity and compare them between Njord and T7 promoters. DNA-terminus initiated transcription was assayed by generating IVT templates which contained either the T7 or Njord promoter at one end, and a sequence ending in a tetraguanosine tract at the other end. The terminal sequence was selected because it is known to be a substrate for promoter-independent transcription by T7 RNA polymerase. Indeed, T7 produces additional species that migrate faster in a native polyacrylamide gel electrophoresis (PAGE) than the expected 300 nt run-off transcript (FIG. 5A). The multiple fast-migrating bands can together be assigned as dsRNA of size ˜300 bp by their sensitivity to RNAse III digestion, resistance to RNAse If digestion, and ability to intercalate acridine orange dye. Cold-active RNA polymerase produces nearly undetectable levels of these dsRNA species.

Absolute dsRNA content in IVT mRNA is typically measured using monoclonal antibodies, either in a dot-blot assay or an enzyme-linked immunosorbent assay (ELISA). Here we adapted a homogeneous time resolved fluorescence (HTRF) kit designed for quantification of dsRNA content in virus-infected cells (Anal. Biochem. 566, 46-49 (2019)). This assay uses two dsRNA-specific antibodies tagged with either a donor or acceptor fluorophore and measures fluorescence resonance energy transfer when both bind the analyte. The assay was employed for qualitative assessment of dsRNA content in IVT RNA produced by the two RNA polymerases using multiple templates. RNA produced with cold-active RNA polymerase clearly has a lower proportion of dsRNA compared to RNA produced with T7 RNA polymerase (FIG. 5B). At the maximum input of cold-active RNA polymerase the HTRF signal is low, and its response to dilution is suggestive of dsRNA species shorter than ˜100 bp. These results are consistent with the absence of DNA-terminus initiated transcription activity in cold-active RNA polymerase and indicate that long dsRNA molecules may be the principle immunostimulatory species in IVT RNA, as previously suggested.

### EXAMPLE 14: Expression and Immunogenicity of mRNA Made by a Cold-Active RNA Polymerase in Cell Culture

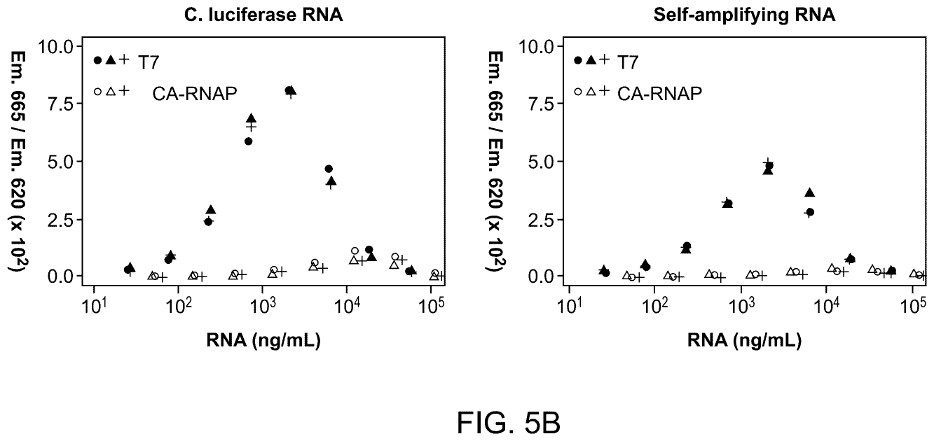

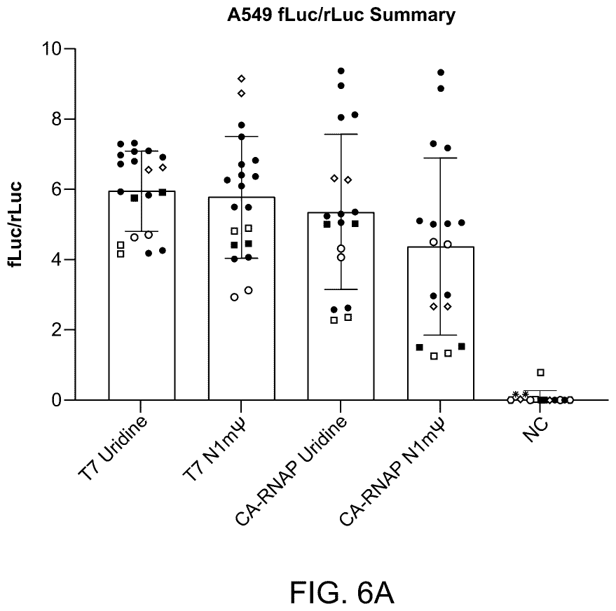

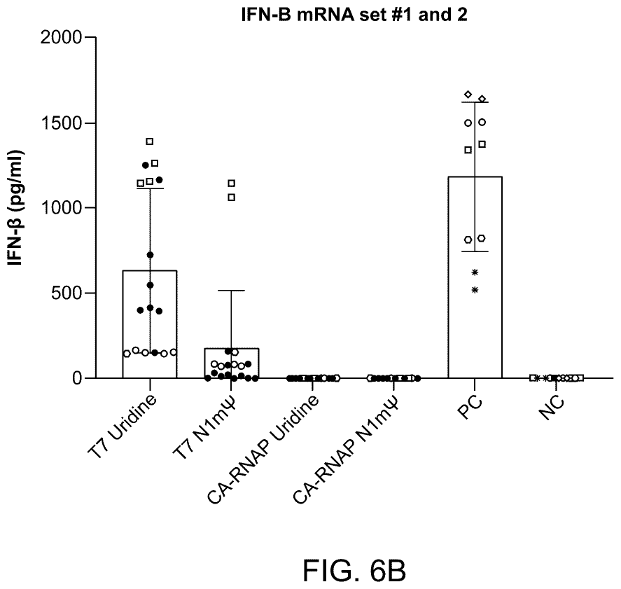

In mammalian cells, the innate immune system can sense dsRNA species and mount an immune response. The immune response can take different forms, including the inhibition of ribosomal translation and the production of pro-inflammatory cytokines. The low dsRNA content observed in IVT reactions suggests that cold-active RNA polymerase may be specifically well-suited for synthesizing mRNA. To test this hypothesis, mRNA encoding the firefly luciferase gene (SEQ ID NO:50) was synthesized using either T7 or cold-active RNA polymerase with unmodified uridine or NlmY. The mRNA was enzymatically capped using the Faustovirus capping enzyme and purified. These mRNAs were introduced into A549 cell culture by transfection with a lipid reagent, and expression of the encoded luciferase was measured alongside the degree of cytokine induction. Regardless of the polymerase or nucleotide modifications used, the expression of the luciferase mRNA relative to a standard control mRNA was comparable (FIG. 6A). This indicates that both RNA polymerases produce similar levels of translatable, in-tact mRNA. In contrast, both nucleotide modification and polymerase strongly affected the degree of cytokine induction (FIG. 6B). When N1mψ is fully incorporated into the mRNA, the amount of interferon beta (IFN-B) excreted by the lung cancer cells is reduced, and when cold-active polymerase is used in place of T7 RNA polymerase, the IFN-β levels are below the detection limit of the assay. These results clearly demonstrate cold active RNA polymerase produces less immunostimulatory byproducts than the current state-of-the-art polymerase.

### EXAMPLE 15: Expression and Immunogenicity of Luciferase mRNA Made by Cold-Active RNA Polymerase in Live Mice

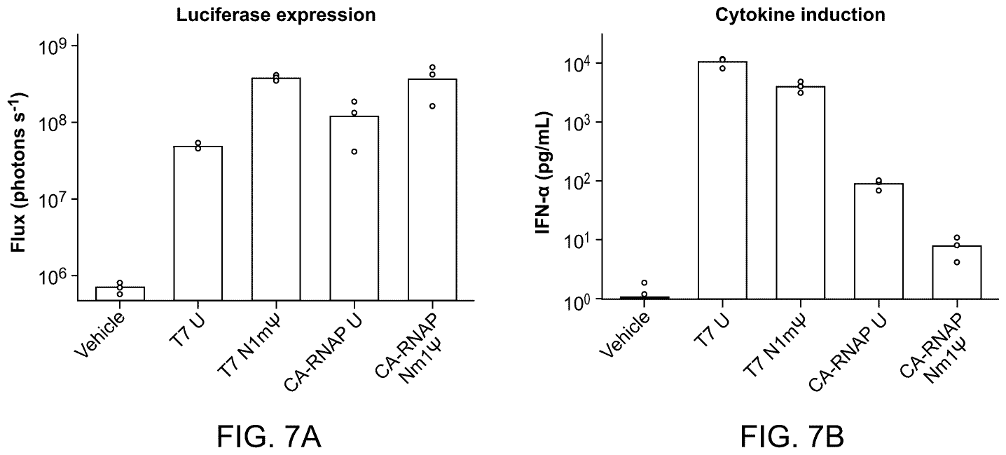

Immunostimulatory properties of mRNA produced by the cold active RNA polymerase in cell culture motivated additional experiments in living animals. Cell culture cannot fully recapitulate the complexities of the innate immune system. Mouse models were employed to confirm the trends in cytokine induction by mRNA produced using different polymerases, with or without the modified nucleotide N1mψ. Firefly luciferase mRNA (SEQ ID NO:49) synthesized by T7 or cold active RNA polymerases was complexed with a commercial reagent to form lipid nanoparticles containing the mRNA. These test articles were delivered to mice by injection to the dorsal tail vein at two different dosages. Six hours later, mice in the low-dose arm of the study were injected with luciferin. Localization of luciferase and the degree of its expression were quantified by in vivo imaging (FIG. 7A). All test articles localized to the liver as expected. Inclusion of N1mψ was critical for high levels of expression regardless of the polymerase used. In the high-dose arm of the study, mice were sacrificed six hours after injection of the mRNA lipid nanoparticles, serum was collected, and levels of circulating interferon alpha (IFN-A) were measured (FIG. 7B). As in cell culture, both nucleotide modification and RNA polymerase had a significant effect on cytokine induction, with N1my and the cold active RNA polymerase each independently reducing the degree of immune system stimulation. Whether or not modified nucleotides were used, replacement of T7 RNA polymerase with the cold active polymerase described here reduced IFN-A stimulation by multiple orders of magnitude. When cold active polymerase is used together with NlmY, IFN-A is only barely detected. These data are consistent with results from cell culture and clearly demonstrate the superiority of cold active polymerase to current methods in mRNA synthesis.

### EXAMPLE 16: Co-Transcriptional Capping with Cold-Active RNA Polymerase, a Capping Enzyme, and a Fusion Enzyme

A cold-active RNA polymerase may be combined with a capping enzyme in an appropriate buffer containing S-adenosyl methionine (SAM) for installation and methylation of the 5′ guanosine cap. To promote co-transcriptional capping, a capping enzyme may be fused to the cold-active RNA polymerase. A fusion enzyme of Faustovirus capping enzyme linked to a cold-active RNA polymerase was designed with an example linker peptide (SEQ ID NO: 20).

The fusion enzyme was overexpressed in E. coli cells and purified according to a miniaturized version of the method described for the cold-active RNA polymerase (Example 3). Co-transcriptional capping RNA synthesis reactions were carried out by combining the fusion enzyme in reaction buffer with an appropriate DNA template, NTPs and SAM at 25° C. RNA reactions products were purified and subjected to RNase 4 digestion. Following digestion, 5′ RNA fragments were further affinity purified using a specific biotinylated oligo and analyzed by gel electrophoresis for the incorporation of the 5′ guanosine cap. The protocol for measurement of 5′ cap incorporation is described in greater detail in Example 7, except that here denaturing polyacrylamide gel electrophoresis is used in place of mass spectrometry to resolve the uncapped and capped 5′ RNA fragments.

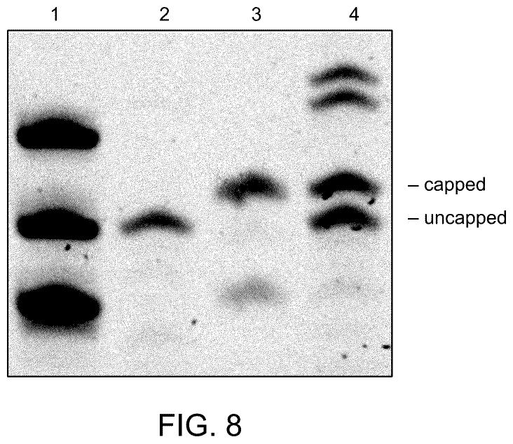

Co-transcriptional capping by a Faustovirus capping enzyme and cold-active RNA polymerase fusion is demonstrated in FIG. 8. Lane 1 contains a set of RNA standards of size 17, 21 and 25 nucleotides. In lane 2, the 5′ fragment of an RNA produce by cold-active RNA polymerase is shown. This corresponds to the region of RNA that is protected from RNase 4 by annealing with a biotinylated DNA oligo and serves as a standard for the uncapped RNA product. Similarly, the single RNA species in lane 3 was prepared by sequentially transcribing the RNA and then capping it with Faustovirus capping enzyme in separate reactions. Slower migration in the acrylamide gel reflects the addition of the 5′ guanosine cap nucleotide. Co-transcriptional capping by the fusion enzyme is represented in lane 4, where both 5′ uncapped and capped RNA fragments are visible. Slower-migrating bands present in lane 4 likely correspond to incomplete digestion of both capped and uncapped RNA species by RNase 4. Additional bands corresponding to larger RNA species have been omitted from FIG. 8 and these are also assigned to partially digested RNA fragments. The results shown demonstrate the ability of the fusion enzyme to perform co-transcriptional capping and to generate mRNA suitable for transfection in a mammalian cell in a single reaction.

1. A composition comprising:
a cold-active RNA polymerase having an amino acid sequence at least 95% identical to any of SEQ ID NOS: 1-16, 18, and 19; and
a capping enzyme,
wherein the cold-active RNA polymerase is a non-naturally occurring cold-active RNA polymerase or a cold-active RNA polymerase of Pseudomonas phage Njord, Pseudomonas phage Nerthus, Pseudomonas phage Alpheus, Pseudomonas phage Achelous, Pseudomonas phage uligo, Vibrio phage φA318, Vibrio phage qAS51, Vibrio phage Vp670, Vibrio phage Vc1, Vibrio phage VEN, Marinomonas phage CPP1m, Marinomonas phage CB5A, Pseudomonas phage Ulina01, Pseudomonas phage Ulitu01, or Pseudomonas phage BUCT553, and the capping enzyme is a non-naturally occurring capping enzyme or a capping enzyme of Faustovirus, mimivirus, or moumouvirus.

2. A composition according to claim 1, wherein the polymerase comprises at least one conservative substitution relative to SEQ ID NO:1.

3. A composition according to claim 1, wherein the amino acid sequence of the polymerase is less than 100% identical to SEQ ID NOS: 1-15 and/or wherein the amino acid sequence of the polymerase is 100% identical to SEQ ID NO:17.

4. A composition according to claim 1, wherein the polymerase is immobilized to a support or the capping enzyme is immobilized to a support or the polymerase and the capping enzyme are each immobilized to a separate support or the polymerase and the capping enzyme are each immobilized to a common support.

5. A composition according to claim 1, wherein the polymerase and the capping enzyme are included in a fusion comprising, in an N-terminal to C-terminal direction, (a) the polymerase and the capping enzyme or (b) the capping enzyme and the polymerase.

6. A composition according to claim 5, wherein the fusion further comprises a linker between the polymerase and the capping enzyme.

7. A composition according to claim 1 further comprising one or more of:
guanosine triphosphate (GTP) or modified GTP;
a methyl group donor;
a 2′ O-methyltransferase; and
a buffering agent.

8. A composition according to claim 1 further comprising a polynucleotide template comprising, in a 5′ to 3′ direction, a promoter corresponding to the polymerase and a sequence of interest.

9. A composition according to claim 8, wherein the promoter has a nucleotide sequence according to one of SEQ ID NOS: 31-46 and/or wherein the sequence of interest comprises a coding sequence.

10. A composition according to claim 1 further comprising a polyribonucleotide product of the polymerase, wherein the product has fewer double-stranded RNA molecules than a polyribonucleotide product of T7 RNA polymerase having the same nucleotide sequence.

11. A composition according to claim 1 further comprising at least one of a buffering agent and/or a polyamine.

12. A composition according to claim 11, wherein the buffering agent comprises HEPES, MES, MOPS, TAPS, tricine, Tris, ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, or triethanolamine.

13. A composition according to claim 11, wherein the polyamine comprises spermidine, spermine, putrescine, polyethylenimine, 1,4,7-triazacyclononane, cyclen, ethylenediamine, or 1, 3, 5,-triazinane.

14. A fusion comprising:
(a) in an N-terminal to C-terminal direction:
(i) a polymerase, wherein the polymerase
(1) is a non-naturally occurring cold-active RNA polymerase or a cold-active RNA polymerase of Pseudomonas phage Njord, Pseudomonas phage Nerthus, Pseudomonas phage Alpheus, Pseudomonas phage Achelous, Pseudomonas phage uligo, Vibrio phage qA318, Vibrio phage qAS51, Vibrio phage Vp670, Vibrio phage Vc1, Vibrio phage VEN, Marinomonas phage CPP1m, Marinomonas phage CB5A, Pseudomonas phage Ulina01, Pseudomonas phage Ulitu01, or Pseudomonas phage BUCT553; and
(2) has an amino acid sequence at least 95% identical to any of SEQ ID NOS: 1-16, 18, and 19; and
(ii) a capping enzyme, wherein the capping enzyme is a non-naturally occurring capping enzyme or a capping enzyme of Faustovirus, mimivirus, or moumouvirus; or
(b) in an N-terminal to C-terminal direction, the capping enzyme and the polymerase.

15. A fusion according to claim 14, wherein:
(a) the amino acid sequence of the polymerase has at least 98% identity to SEQ ID NO:1;
(b) the amino acid sequence of the capping enzyme has at least 98% identity to SEQ ID NO: 75; or both (a) and (b).

16. A fusion according to claim 14, wherein the fusion is immobilized to a support.

17. A fusion according to claim 14 further comprising, a purification tag or a sorting signal peptide, wherein the fusion comprises:
in an N-terminal to C-terminal direction, the purification tag or the sorting signal, the polymerase, and the capping enzyme, or
in an N-terminal to C-terminal direction, the purification tag or the sorting signal, the capping enzyme and the polymerase.

18. A kit comprising:
a fusion according to claim 14; and
one or more of:
guanosine triphosphate (GTP) or modified GTP;
a methyl group donor;
a 2′ O-methyltransferase; and
a buffering agent.

19. A method comprising:
(a) contacting an RNA polymerase at least 95% identical to any of SEQ ID NOS: 1-16, 18, and 19 and a polynucleotide template comprising an expression control sequence of the RNA polymerase and a coding sequence operably linked to the expression control sequence to produce an elicitor transcript that is heterologous to and/or translatable by a mammalian cell; and
(b) contacting the elicitor transcript with the mammalian cell to form a translation product of the elicitor transcript,
wherein the mammalian cell produces smaller quantities of at least one cytokine than a reference mammalian cell comprising a reference transcript, wherein the reference transcript is produced by contacting T7 RNA polymerase with the same polynucleotide template.

20. A method according to claim 19, wherein the translation product has a therapeutic effect on the cell.

21. A method according to claim 19, wherein the mammalian cell is contiguous with and/or in communication with other mammalian cells in a mammal.

22. A method according to claim 19, wherein the elicitor transcript comprises a therapeutic RNA or a vaccine RNA.

23. A method according to claim 19, wherein the elicitor transcript comprises is an artificial transcript.

24. A method comprising:
(a) contacting:
(i) an RNA polymerase at least 95% identical to any of SEQ ID NOS: 1-16, 18, and 19;
(ii) a polynucleotide template comprising an expression control sequence of the RNA polymerase and a coding sequence encoding an artificial transcript, the coding sequence operably linked to the expression control sequence; and
(iii) ribonucleotide triphosphates,
to produce the artificial transcript.

25. A method according to claim 24 further comprising:
(b) contacting the artificial transcript with a capping enzyme and one or more of (i) guanosine triphosphate (GTP) or modified GTP, (ii) a methyl group donor, (iii) a 2′ O-methyltransferase, and (iv) a buffering agent,
to produce a capped artificial transcript.
